(19) United States
(12) Patent Application Publication
(10) Pub. No.: US 2026/0294922 A1
(43) Pub. Date: Oct. 1, 2026

(54) Inhibitors of JUN N-Terminal Kinases (JNK1, JNK2, and/or JNK3) and Mitogen-Activated Protein Kinases (MAPK8, MAPK9, and/or MAPK10) and Methods of Using Same

(71) Applicant: Baylor College of Medicine, Houston, TX (US)

(72) Inventors: Chandra Madasu, Houston, TX (US); Stephen Palmer, Houston, TX (US); Martin Matzuk, Houston, TX (US); Diana Monsivais, Houston, TX (US); Kurt Bohren, Houston, TX (US); Zhi Tan, Houston, TX (US); Jian-Yuan Li, Houston, TX (US); Feng Li, Houston, TX (US); Damian Young, Houston, TX (US); Kiran Sharma, Houston, TX (US); Murugesan Palaniappan, Houston, TX (US); Srinivas Chamakuri, Houston, TX (US); Ramakrishna Kommagani, Houston, TX (US)

(21) Appl. No.: 19/478,160

(22) PCT Filed: Apr. 24, 2024
(86) PCT No.: PCT/US2024/025997

§ 371 Date: Oct. 23, 2025

Related U.S. Application Data
(60) Provisional application No. 63/461,485, filed on Apr. 24, 2023.

(51) Int. Cl. A61K 31/5377; A61K 31/437; A61K 31/444; A61K 31/4545; A61K 31/4725; A61K 31/496; A61K 31/506; A61K 31/57; A61P 15/00; A61P 29/00; C07D 241/04; C07D 471/04; C07D 519/00

(52) U.S. Cl. CPC A61K 31/5377; A61K 31/437; A61K 31/444; A61K 31/4545; A61K 31/4725; A61K 31/496; A61K 31/506; A61K 31/57; A61P 15/00; A61P 29/00; C07D 241/04; C07D 471/04; C07D 519/00

(57) ABSTRACT

The present disclosure relates, in part, to compounds of Formula (I) and (II), which selectively inhibit JUN N-Terminal Kinases (JNK1, JNK2, and/or JNK3; also known as MAPK8, MAPK9, and/or MAPK10), pharmaceutical compositions thereof, and methods of using the same for the treatment, prevention, and/or amelioration of one or more diseases and/or disorders in a subject. In certain embodiments, the inflammatory disease or disorder is endometriosis, arthritis, pulmonary fibrosis, cancer, type 1 and/or 2 diabetes, Alzheimer's disease, Parkinson's disease, or amyotrophic lateral sclerosis. In certain embodiments, the methods described herein further comprise detecting the disease and/or disorder in the subject with a suitable diagnostic method.

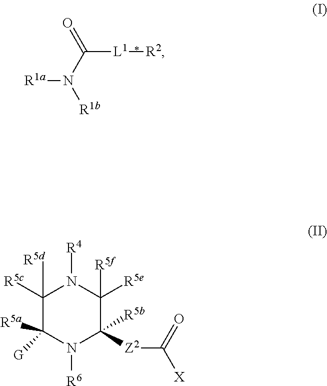

### CROSS-REFERENCE TO RELATED APPLICATIONS

This application claims priority under 35 U.S.C. § 119(e) to U.S. Provisional Patent Application No. 63/461,485, filed Apr. 24, 2023, which is incorporated herein by reference in its entirety.

### STATEMENT REGARDING FEDERALLY SPONSORED RESEARCH OR DEVELOPMENT

This invention was made with government support under R01HD099341-OIAI and R01HD110038 awarded by the Eunice Kennedy Shriver National Institute of Child Health and Human Development (NICHD). The government has certain rights in the invention.

### BACKGROUND

Endometriosis is a common morbid disease that affects approximately 10-15% of women worldwide. Pain and infertility are the two major clinical diagnoses from which physicians select therapeutic treatments for their patients. In 2012, the average annual cost per endometriosis patient to seek relief from pain was reported to be nearly $12,000 higher than for women without endometriosis: of that cost, nearly $4,000 per year were out-of-pocket medical care costs, with the remainder attributed to physician care costs.

Currently, anti-endometriosis therapies seeking approval are directed towards suppression of estrogen production or inhibition of estrogen receptor activation, and through this mechanism it is expected that lesions regress and pain is reduced. Nevertheless, lesions remain, and pain continues to be a complaint among patients treated with therapies that disrupt the endocrine axis.

There are no alternative anti-endometriosis therapies that address fundamental pathological mechanisms associated with the disease without interrupting the endocrine system. Innovative therapies that offer new and complementary approaches to cause regression of lesions, minimize pain, and potentially restore fertility after therapy would provide for current unmet needs.

Inhibition of one or more JUN N-Terminal Kinases (i.e., JNK1, JNK2, and/or JNK3), also known as Mitogen-Activated Protein Kinases (i.e., MAPK8, MAPK9, and/or MAPK10), have already been validated as targets in non-human primates and humans, and can allow for disrupting endometriotic lesion survival and pain without impacting normal ovarian and endometrial function. Existing JNK inhibitors have not continued into late stage clinical trials for endometriosis, and it is unknown whether this is due to a lack of efficacy, safety concerns, or simply deprioritization of endometriosis drug discovery efforts.

Thus, there is a need in the art for selective inhibitors of JNK proteins and methods of thereof. The present disclosure addresses this need and further identifies JNK-Is with more desirable target modulation profiles than any such compounds known in the art, if any.

### BRIEF SUMMARY

In one aspect, the disclosure provides a compound of Formula (I), or a salt, solvate, stereoisomer, or isotopologue thereof, wherein R^{1a}, R^{1b}, L¹, and R² are defined elsewhere herein.

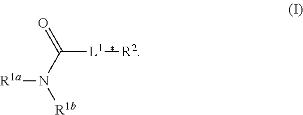

In certain embodiments, the compound of formula (I) is a compound of formula (Ia), wherein R^{1a}, R^{1b}, R², R^{3a}, R^{3b}, R^{3c}, and R^{3d} are defined elsewhere herein:

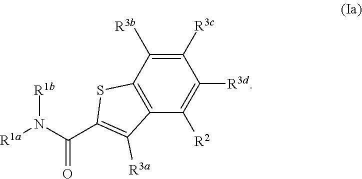

In certain embodiments, the compound of formula (I) is a compound of formula (Ib), wherein R^{1a}, R^{1b}, R², R^{3a}, R^{3b}, R^{3c}, and R^{3d} are defined elsewhere herein:

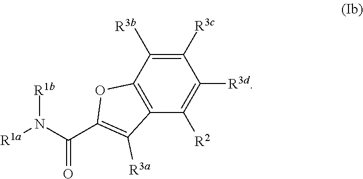

In certain embodiments, the compound of formula (I) is a compound of formula (Ic), wherein R^{1a}, R^{1b}, R², R^{3b}, R^{3c}, and R^{3d} are defined elsewhere herein:

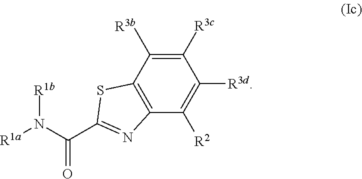

In certain embodiments, the compound of formula (I) is a compound of formula (Id), wherein R^{1a}, R^{1b}, R², R^{3a}, R^{3b}, R^{3c}, and R^{3d} are defined elsewhere herein:

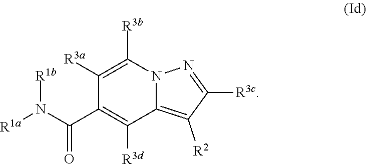

In certain embodiments, the compound of formula (I) is a compound of formula (Le), wherein R^{1a}, R^{1b}, R², R^{3a}, R^{3b}, R^{3c} and R^{3d} are defined elsewhere herein:

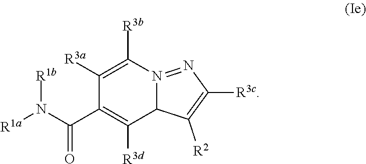

In certain embodiments, the compound of formula (I) is a compound of formula (If), wherein R^{1a}, R^{1b}, R², R^{3a}, R^{3b}, and R^{3d} are defined elsewhere herein:

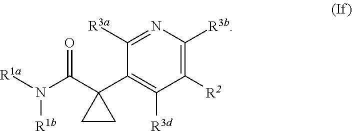

In certain embodiments, the compound of formula (I) is a compound of formula (Ig), wherein R^{1a}, R^{1b}, R², R^{3a}, R^{3b}, R^{3c}, and R^{3d} are defined elsewhere herein:

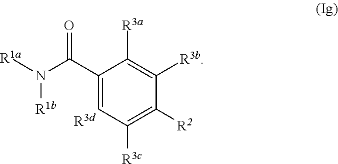

In another aspect, the disclosure provides a compound of Formula (II), or a salt, solvate, stereoisomer, or isotopologue thereof, wherein R⁴, R^{5a}, R^{5b}, R^{5c}, R^{5d}, R^{5c}, R^{5f}, R⁶, G, X, and Z² are defined elsewhere herein:

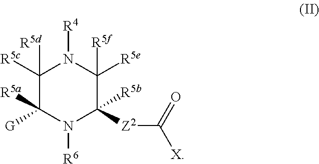

In another aspect, the disclosure provides a pharmaceutical composition comprising at least one compound of the disclosure and a pharmaceutically acceptable carrier.

In another aspect, the disclosure provides a method of treating, preventing, and/or ameliorating an inflammatory disease in a subject. In certain embodiments, the method comprises administering to the subject a therapeutically effective amount of at least one compound of the disclosure and/or a pharmaceutical composition of the disclosure. In certain embodiments, JNK1 is inhibited at a similar or greater rate of inhibition than JNK2 and/or JNK3.

In certain embodiments, the inflammatory disease is a non-central nervous system (CNS) inflammatory disease. In certain embodiments, the inflammatory disease is at least one selected from the group consisting of endometriosis, arthritis, pulmonary fibrosis, cancer, type 1 diabetes, and type 2 diabetes.

### BRIEF DESCRIPTION OF THE FIGURES

The drawings illustrate generally, by way of example, but not by way of limitation, various embodiments of the present application.

FIG. 1 depicts the use of DEC-Tec for the discovery of novel JNK inhibitors (JNK-I). Key steps for using DEC-Tec to discover JNK-I include: 1) screening libraries of >4 billion DNA-encoded drug-like compounds, each with a “bar code”; 2) affinity selection of the library against his-tagged JNK1, JNK2, and JNK3; 3) isolation of small molecules bound to the kinase(s) and beads; 4) next-generation sequencing and informatics analysis of the DNA bar codes to decode the molecular structures of putative selective, drug-like JNK-I “Hits”; 5) synthesis of the drug-like “Hits” without the DNA tag; and 6) analysis of optimized hits in vitro and in vivo.

FIG. 2 depicts DEC-Tec selection results with JNK1 at 0.05 μM compared to a no target control. Bentamapimod (10 μM) was used as a competitor to distinguish specific and non-specific binders.

FIGS. 3A-3B depict DEC-Tec a close up view of the selection results for qDOS21 (FIG. 3A) and qDOS28_1 (FIG. 3B) for JNK3 (0.05 μM) compared to no target control. Bentamapimod (10 μM) was used as a competitor to distinguish specific and non-specific binders. Three building blocks include C1 (bottom), C2 (middle), and C3 (top). FIG. 3A: important features of the qDOS21 library include the high count and score criteria that provide additional measures of relatedness of the hits (e.g., C1, C2) beyond only the enrichment score. By comparison, hits from qDOS28_1 had much lower enrichment, lower counts, and lower scores. Bentamapimod (10 M) was used as a competitor to distinguish specific and non-specific binders for all assays. FIG. 3B: enrichment of building block C3 was obtained from qDOS28_1 with various C1 and C2 building blocks. The enrichment of each hit series is indicated below each scaffold as count/z-score at 0.05 μM.

FIGS. 4A-4D depict docking of highly enriched compounds from the qDOS21 library onto the JNK3 crystal structure: CDD-2728-JNK1 (PDB: 4L7F) interaction (FIG. 4A); CDD-2728-JNK3 (PDB: 4WHZ) interaction (FIG. 4B); CDD-3013-JNK1 (PDB: 4L7F) interaction (FIG. 4C); and CDD-3013-JNK3 (PDB: 4WHZ) interaction (FIG. 4D). In the models depicted herein, a different orientation of CDD-2728 (FIGS. 4A-4B) and CDD-3013 (FIGS. 4C-4D) in JNK1 (MAPK8; FIG. 4A and FIG. 4C) relative to JNK3 (MAPK10; FIG. 4B and FIG. 4D) is observed.

FIGS. 5A-5C depict KINOMEscan selectivity profile of Staurosporine (FIG. 5A), Bentamapimod (BEND; FIG. 5B) and Tanzisertib (TANZ; FIG. 5C) at 1 μM for 480 kinases. JNK1-3 (CMGC family), were inhibited by BEND and to a greater extent by TANZ. While both JNK-I were highly selective for JNK (spots at 7:00 position), tanzisertib weakly inhibited LATS1 (spot at 9:00 position). Inhibition of LATS1 was not confirmed in a subsequent ThermoFisher Z'Lyte assay. Both Bentamapimod and Tanzisertib were deemed safe in populations of volunteers in Phase 1 clinical trials.

FIG. 6A depicts a dendrogram summarizing the selectivity of CDD-2346 (an early analogue obtained from the DEC-Tec selection) over the KINOMEscan. FIGS. 6B-6C depict concentration-dependent evaluation of CDD-2346 inhibition among the “in-pathway” MEK/JNK enzymes (FIG. 6B) and “out-of-pathway” (FIG. 6C) kinases, determined in ThermoFisher Lantha Screen. FIG. 6D depicts concentration-dependent evaluation of CDD-2346 inhibition among some of the additional “out-of-pathway” kinases determined in ThermoFisher TF Z'LYTE assay. Certain exemplary compounds described herein were found to possess a selectivity profile more favorable than that of CDD-2346.

FIG. 7A provides a dendrogram depicting selectivity and efficacy of optimized JNK-I CDD-2728 in the Eurofins KINOMEscan. JNK enzymes are depicted at 7:00 position, and MEK4 at the 2:00 position. FIGS. 7B-7C depict concentration-dependent inhibition of “in-pathway” (FIG. 7B) and “out-of-pathway” (FIG. 7C) kianses by CDD-2728 in ThermoFisher LanthaScreen (FIG. 7B) and in ThermoFisher Z'LYTE assays (FIG. 7C).

FIG. 8: the definition of “in-pathway” relative to “out-of-pathway” followed the common association among the MAPK family with MEK4/7 serving as the upstream regulators of JNK enzymes and MEK1/2 as upstream regulators of ERK1/2 kinases. Enzymes outside of this MAPK family were designated “out-of-pathway”.

FIG. 9 depicts anticipated non-limiting cellular responses for CDD-JNK inhibitors (Bora and Yaba et al. J. Obstet. Gynaecol. Res. 2021, 47(5):1610-1623). Substantiates the “in-pathway” and “out-of-pathway” targets and highlights the anticipated non-limiting effects of JNK-I on inflammation, differentiation, apoptosis, and growth, but reduced expectation of effect on cellular proliferation, cellular division, and cell cycle arrest as anticipated properties of selective JNK-I.

FIGS. 10A-10B represents data generated in a Promega NanoBRET system to confirm that JNK-I described herein are effective in the intracellular environment where they are anticipated to be effective modulators of endometriosis pathologies. The graphs depict displacement of tracer K10 by several CDD-JNK-I in Promega NanoBRET for CDD hits in JNK1 assays (FIG. 10A) and JNK3 assays (FIG. 10B). FIG. 10C: activity of optimized inhibitors CDD-2728, CDD-2729, and CDD-2731 on Promega NanoBRET systems expressing JNK1. The calculated IC₅₀ values for CDD-2728, CDD-2729. CDD_2731 are further provided in FIG. 10C. FIG. 10C illustrates the activity of earlier analogues identified in the program for activity in NanoBRET JNK3 cells.

FIG. 11: CDD-3013 was evaluated in a Promega NanoBRET panel of 192 kinase assays. The illustration describes the principle of the BRET-based ligand displacement assay conducted in live cells.

FIGS. 12A-12J provide graphs showing that CDD-2628 inhibits IL-1b-stimulated pro-inflammatory biomarker responses in 12Z endometriotic cells with similar potency as bentamapimod (i.e., FIGS. 12A-12B and FIGS. 12E-12F) or with improved potency (IL-8, FIGS. 12C-12D; MMP-3, FIGS. 12G-12H) relative to bentamapimod (CDD-939). Similar effects of CDD-2575 and CDD-2634 were observed for the biomarker MMP-3.

FIGS. 13A-13B provide graphs from replicate experiments showing that CDD-2728 and CDD-2634 are selective JNK inhibitors that are more potent inhibitors of IL-8 expression than bentamapimod (CDD-939).

FIGS. 14A-14H provide graphs from replicate experiments showing that CDD-2728 inhibits IL-1β-stimulated pro-inflammatory biomarker responses in 12Z endometriotic cells with similar potency as bentamapimod (CDD-939). Replicating previous observations, CDD-2728 continues to be more potent than CDD-939 at inhibiting MMP-3 expression. FIGS. 14G-14H represent responses of primary human endometriotic stromal cells [from the endometrioma lesion] from endometriosis patient “50”. CDD-2728 represented an effective inhibitor of IL-6 gene expression in these cells than CDD-939.

FIGS. 15A-15H provide replicate results of the response of primary human endometriotic stromal cells (derived from the lesions) obtained from endometriosis patient “50”. In each example. CDD-2728 was a more effective inhibitor of the biomarkers IL-8, MMP-3, and PTGS2 gene expression than CDD-939. In each example, CDD-2728 inhibited IL-1β-stimulated pro-inflammatory biomarker responses in primary endometriotic stromal cell cultures. Lower concentrations of JNK inhibitors were required to reduce the proinflammatory response of IL-1β in stromal cells than the concentration of JNK-I in 12Z epithelial cells.

FIGS. 16A-16F provide results obtained from cultures of human endometriotic stromal cells (i.e., from the endometrioma lesion) obtained from a second endometriosis patient “143”, showing that CDD-2728 inhibits IL-1β-stimulated IL-8, MMP-3, PTGS2 gene expression at lower doses than was required for inhibition of expression of these genes by CDD-939. Similar to patient “50”, lower concentrations of CDD-2728 were required to reduce the proinflammatory response of IL-1β in stromal cells on these measured genes than in 12Z epithelial cells.

FIGS. 17A-17C provide bar graphs depicting ATP consumption (% viability) of HepG2 cells with administration of CDD-JNK inhibitors CDD-2575 (FIG. 17A), CDD-2728 (FIG. 17B), and CDD-2634 (FIG. 17C). This assay reflects cell viability. A decrease in cell viability suggests a non-specific effect of the inhibitor on cell survival. The results suggest that non-specific decreases in cell viability were pronounced for CDD-2575 at 25 μM, while higher concentrations of CDD-2728 were tolerated (up to 75 μM). Continued improvements in JNK-I selectivity and permeability were obtained with CDD-3013, in which no decrease in HepG2 cell viability was observed at 100 M.

FIG. 18 provides bar graphs depicting ATP consumption (% viability) of HepG2 cells with administration of JNK-I CDD-3013 compared to 3 other kinase inhibitors. The results demonstrate no evidence of cytotoxicity in HEPG2 cells up to 100 μM. The dose at which CDD-3013 is estimated to cause cytotoxicity on the basis of the experiments described herein exceeds 100 M (e.g., 143 μM). For comparison, the IC₅₀ for induction of cytotoxicity response for CDD-XX07 is 48 μM, for CDD-XX10 is 34 μM, and for CDD-XX30 is 72 μM. This suggests that a 138-fold safety margin can be obtained with cell culture concentrations of 1 μM, and a 13.8-fold safety margin is obtained at cell culture concentrations of 10 μM CDD-3013.

FIGS. 19A-19B provide results of pharmacokinetic analysis of concentrations of CDD-2728 measured in plasma following oral administration of CDD-2728 and the rate of clearance in mice with oral (FIG. 19A) or intraperitoneal (FIG. 19B) administration of 50 mg/kg CDD-2728 in a carboxy-methylcellulose (CMC)-sodium formulation.

FIG. 20 depicts an enrichment profile of compounds identified in qDOS 11 that bind to JNK1 present at a concentration of 0.3 μM. In qDOS 1l, where the methyl amide in BB1 represents the DNA attachment point. A series of hits were identified with the same BB1 (blue) and BB3 (black) with a variety of BB2 (red) as well as the di-synthon enrichment of the same BB2 and BB3. Structure-enrichment relationship was observed with the BB2 of the hit with highest z-score (i.e., 6.3).

FIG. 21A depicts results of a JNK1 biochemical assay with compounds CDD-1722, CDD-1723, CDD-2009, CDD-2010 and CD-985 (tanzisertib). FIG. 21B depicts concentration-dependent inhibition of JNK1 with CDD-1722 and CDD-1723. Components of CDD-1722 and CD-1723 represent alternative building blocks which may be incorporated during optimization of pharmaceutical properties of JNK-I.

FIG. 22A presents the design of an in vivo model in which JNK-I were evaluated for their ability to cause regression of endometriosis in mice. Results of an in vivo disease-relevant model of endometriosis in mice are depicted. In this experimental design, endometriosis is induced by introducing isolated endometrial tissues from a donor mouse, and the prepared endometrial cells are introduced into the peritoneal cavity of recipient mice. Fourteen (14) days after induction of endometriosis, drug treatments are initiated and continued for 14 days. At the end of study (i.e., 28 days) the presence of endometriosis lesions are determined and various measurements performed. FIG. 22B depicts the results obtained with CDD-2728 utilizing the model of endometriosis described herein. CDD-2728 decreases the number of endometriotic lesions, causes reduction in the size of lesions remaining in the mice (mm³), and the mass of the lesions (mg). There was no impact of CDD-2728 on uterine weight, liver weight or kidney weight suggesting a specific effect on the lesion without impacting the uterus or other organs.

FIG. 23 depicts immunofluorescent staining of sections of remaining endometriotic lesions following 14 days of treatment with CDD-2728 or with a vehicle solution (i.e., Captisol) by intraperitoneal administration. Results from these images indicate that JNK-I CDD-2728 causes reduced rates of proliferative cell growth of lesions (Ki67), reduces the presence of cells corresponding to leukocytes, platelets, and endothelial cells (CD31), and decreases infiltration of endometriotic lesions by macrophages (F4/80).

FIGS. 24A-24E depict the effects of CDD-3013 on regression of endometriotic lesions per mouse (FIG. 24A), volume of lesions (FIG. 24B), mass of lesions (FIG. 24C), and show that the impact of CDD-3013 on endometriotic lesions did not impact other tissues such as liver (FIG. 24D) or kidney (FIG. 24E), demonstrating a unique impact on the site of inflammation. Endometriotic lesions (numbers, volumes, and lesion mass) are significantly reduced with CDD-3031 treatment of mice at 10 mg/kg.

FIG. 25 shows that treatment of mice bearing endometriotic lesions caused a change in the rate of proliferation of endometriotic cells, a decrease in the recruitment of endothelial cells (CD31) into lesions, and a decrease in the recruitment of macrophages towards lesions. This illustrates a common mechanism of JNK-I from the chemical series of the disclosure and their ability to target endometriotic lesions, and the innate immune cells that drive an inflammatory response within the lesions. Endometriotic lesions from mice treated with CDD-2728 showed reduced cellular proliferation (anti-Ki67 antibody), angiogenesis (anti-CD31 antibody), and macrophage infiltration (anti F4/80 antibody).

FIG. 26A depicts the effects of CDD-2728 and CDD-3013 on phosphorylation of the JNK enzyme itself (i.e., representing MEK4 inhibition) and phosphorylation of pSer63 and pSer73 on the transcription factor Jun in immortalized human endometriotic epithelial cells (iHEEC). The HEEC cells are a well characterized cell line that are relevant for the evaluation of inflammatory endpoints and measures of epithelial-to-mesenchymal transitions (EMT). Relative to CDD-939 (bentamapimod) that does not inhibit MEK4 in KINOMEscan, CDD-2728 inhibits phosphorylation of JNK1 (P46) and JNK2 (P54). In addition, CDD-2728 inhibits Jun at pSer63 and pSer73 more effectively than CDD-939. This is consistent with the potency relationships of CDD-2728 relative to bentamapimod observed in cytokine gene expression described above in iHEEC cells and primary human stromal endometriotic cells. For comparison purposes, a commonly used reference JNK-I (i.e., SP600125) also inhibits pSer63 and pSer73 Jun as well as MEK4-dependent phosphorylation of JNK1 and JNK2. SP600125 has been frequently described as a poorly-selective JNK-I but in this context provides additional confirmation of inhibition of the JNK target kinase for CDD-2728. FIG. 26B depicts the inhibition of pSer63 and pSer73 of Jun by JNK-I CDD-2856, which inhibits phosphorylation more effectively than Tanzisertib, and CDD-2856 inhibits the upstream phosphorylation of JNK1 and JNK2. These results illustrate the common feature of JNK-I disclosed herein (i.e., their ability to inhibit both the Jun transcription factor (primary target) and the immediately upstream regulator of JNK activity, MEK4 (secondary target)), and the lack of efficacy of Tanzisertib that has a poor IC₅₀ for JNK-1 relative to JNK-2. This adds further support to the critical role of JNK-1 to the therapeutic effect of the JNK inhibitors of the disclosure.

FIG. 27A: CDD-2728 and CDD-3013 have both been shown to be more effective inhibitors of Jun phosphorylation than Tanzisertib (CDD-985) in semi-quantitative, in-cell Western blots. The top section of the in-cell Western is pSer73 and the lower section is pSer63. Experiments were performed in triplicate for 3 concentrations (0.1 μM, 1 μM, and 10 μM) FIGS. 27B-27C: graphical summary of the in-cell Western blot is provided for pSer73 (FIG. 27B) and pSer63 (FIG. 27C).

FIGS. 28A-28C depict the impact of JNK inhibition on the cellular morphology and expression of surface marker, vimentin in iHEEC. These results demonstrate that JNK-I CDD-2728 can reduce the intensity of phospho-JNK staining, but there is not a dramatic change in the vimentin staining. Phospho-JNK staining in iHEEC can be either punctate staining in intracellular organelles or disperse staining through the cytoplasmic space of cells.

FIGS. 29A-29C depict the impact of JNK inhibition on the cellular expression of beta-catenin located at the cell surface and throughout the cytoplasm of iHEEC, wherein HEEC cells were treated with vehicle (FIG. 29A), IL-1β (FIG. 29B), or IL-1β and 10 M CDD-2728 (FIG. 29C). The increased cytoplasmic and plasma membrane expression of β-catenin following exposure to IL-1β supports the role of β-catenin in facilitating epithelial-to-mesenchymal transdifferentiation. The cellular morphology is consistent with that of columnar epithelial cells. iHEEC were treated with JNK-I or vehicle for 30 min before addition of IL-1β, followed by incubation of cells for another 15 minutes. Addition of IL-1β over this short time course caused a dramatic change in the expression of beta-catenin in iHEEC endometriotic epithelial cells, and a noticeable change in phospho-JNK throughout the cytoplasm, induced by IL-1β. Beta catenin expression at contact points between cells is increased by IL-1β, and inhibited by CDD-2728. Over the short time course of this experiment there were no apparent changes in cell viability or cell density caused by JNK-I.

FIGS. 30A-30B depict the superior efficacy of CDD-2728 (FIG. 30A) and CDD-3013 (FIG. 30B) relative to bentamapimod on phosphorylation of Jun relative to total Jun in experiments conduct with 12Z cells in the presence of a pro-inflammatory mediator, IL-1β. The results demonstrate the specific inhibition of phosphorylation of Jun without an impact on total Jun (FIG. 30A) or cellular metabolism (GAPDH). Results in FIG. 30B demonstrate undetectable changes in phosphorylation of JNK1 or JNK2 by CDD-2728 or CDD-3013, indicating that inhibition of the dominant target (JNK) does not cause significant accumulation of phosphorylated JNK1 or JNK2. This suggests that inhibition of JNK phosphorylation of Jun does not cause backup of phosphorylated events in this signaling pathway.

FIGS. 31A-31B depict the impact of CDD-3013 on cells obtained from patient derived endometrioma lesions and illustrate a common mechanism in primary cultures of endometrioma-derived stromal cells as has been described above for immortalized epithelial cells in the descriptions above (12Z cells and iHEEC cells). The results in FIG. 31A demonstrate inhibition of Jun phosphorylation in cells incubated with IL-1β and increasing concentrations of CDD-3013 (0.1, 1, 10 μM). Similar impact of JNK inhibition is observed for the eutopic endometrium (EuE) and ectopic endometrioma cells (OMA) for patient 35. In this patient, increased phosphorylation of JNK1 is observed at the highest concentrations tested relative to decreased phosphorylation of Jun. Similar inhibition of Jun phosphorylation were observed in eutopic endometrial cells and ectopic endometrioma-derived cells obtained from patient 92 (FIG. 31B) in the presence of a proinflammatory mediator, IL-1β. For cells obtained from patient 92, there was minimal change in phosphorylation of JNK1 or JNK2, suggesting that there are likely to be anticipated dosage-dependent requirements for patients receiving JNK therapy for endometriosis to achieve optimal suppression of Jun, with minimal impact on phosphorylation of JNK1 or JNK2.

FIGS. 32A-32B demonstrate that CDD-3013 effects are selective among other cellular kinase pathways in the eutopic and ectopic endometrioma cells. For patient 35, there was a remarkable difference in pSmad1,5 between the eutopic endometrium and the endometrioma. Addition of the JNK inhibitor permitted greater phosphorylation of pSmad1,5 in eutopic endometrial-derived cells, but did not inhibit this kinase activity in either eutopic or ectopic cells. Similar reduced phosphorylation of Smad2 and pSmad 3 and Akt were observed in endometrioma-derived cells compared to eutopic cells, and pSmad2, pSmad3, as well and pAkt increased in eutopic tissue while there was no change in phosphorylation status of these signaling proteins were caused by CDD-3013 in the ectopic endometrioma-derived cells. The apparent increased support of morphogenetic responses (pSmad 1,2,3,5) in eutopic cells while suppressing inflammatory pathways (Jun) in both eutopic and ectopic cells suggest that JNK-I of this chemical series impart selective disease modification in lesions, without adversely impacting the eutopic endometrial-derived cells. These results are largely confirmed in patient 92 (FIG. 32B) with minimal impact of JNK-I CDD-3013 on pSmad1.5 in ectopic endometrioma-derived cells (OMA92) without significant impact on eutopic-derived cells. In both patient 35 and patient 92, pSTAT3 status was unaffected.

FIGS. 33A-33B depict phosphorylation of Jun or JNK in endometrial organoids, obtained from a deep infiltrating lesion, compared to endometrial epithelial organoids obtained from the eutopic endometrium of patient 63. Organoids are considered a more physiologically relevant culture conditions than primary epithelial cells cultured on a flat plastic culture dish. Under these more relevant conditions, the results demonstrate that Jun is phosphorylated to a greater extent in the lesion-derived epithelial organoids than in the eutopic tissue-derived organoids. Stimulation with IL-1β leads to an increase in phosphorylation of Jun in both organoid conditions (eutopic, EuE63; lesion from deep infiltrating type of endometriosis, DiE63). FIGS. 33C-33D show that neither levels of JNK, nor phosphorylation of JNK was visibly different between eutopic or lesion organoids. These results suggest that a basal level of Jun phosphorylation is present in DiE epithelial organoids and that a JNK-inhibitor can reduce the level of Jun phosphorylation without significant impact on the activation of the upstream Kinase MEK4 that causes phosphorylation of JNK.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

In one aspect, the present disclosure relates to the discovery of novel JNK inhibitors (JNK-I) which are useful for the treatment of inflammatory diseases, including but not limited to endometriosis and associated pain.

Preclinical and clinical data for bentamapimod (BEND) largely supports each premise with demonstration of efficacy in preclinical and clinical trials. Support for the mechanistic role of JNK-I in modulating inflammation and fibrosis has been demonstrated for tanzisertib (TANZ), a JNK-I developed for treatment of idiopathic pulmonary fibrosis (IPF). TANZ reduced inflammation, EMT, and fibrosis in preclinical models of IPF and in human IPF patients.

However, neither BEND nor TANZ progressed to Phase 3 clinical trials. TANZ was discontinued after adverse liver effect in IPF. Celgene (now Bristol Myers Squibb) has replaced TANZ with CC-90001 [NCT02510937] for IPF, while screening for hepatic injury [NCT03742882] emphasizing their risk-managed optimism for this target. However, a JNK-I for IPF does not require pain amelioration. The reported IC₅₀ values for BEND and TANZ against JNK1, JNK2, and JNK3 are 80, 90, and 230 nM for BEND, and 400, 38, and 32 nM for TANZ.

The present disclosure relates to one or more unexpected and/or innovative discoveries, non-limiting examples including: (1) new chemical scaffolds for kinase inhibition (e.g., JNK1-3): (2) a unique selectivity profile among JNK isoforms designed to limit adverse effects of excessive inhibition of JNK2 and maximize impact on inflammatory pathways (JNK11), and to decrease the threshold of perceived pain in CNS (JNK3): (3) a unique selectivity profile among the broader kinase family that accentuates inhibition of the MEK4/JNK pathway that largely regulates inflammation without severely impacting cell proliferation and/or other MEK family kinases that could lead to adverse effects in animals and/or humans; (4) a JNK-I that can reduce progesterone insensitivity of the host bearing endometriosis lesion; (5) a JNK inhibitor that inhibits epithelial-mesenchymal transitions as well as mesenchymal to epithelial transitions that precede precancer cell formation; and (6) a JNK inhibitor that can inhibit regrowth of surgically excised and chemically treated cancers by reducing the ability of cancer stem cells to reinitiate tumor formation. In one aspect, JNK inhibitors described herein represent a development strategy for kinase inhibitor selectivity profile that has not been reported prior to this disclosure.

In one aspect, the present disclosure relates to the discovery that JNK1-3 are dominant integrators of the pathophysiology underlying endometriosis, including inflammation, epithelial-mesenchymal trans-differentiation (EMT) of endometrial cells, suppression of immune-mediated clearance of endometriotic cells from their ectopic site(s), and sensory integration of pain. The hypothesis that drove this discovery effort is that JNK inhibitors (JNK-I) can address each of these pathologic features of endometriosis.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms.

Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C—CH, —C═C(CH₃), —C—C(CH₂CH₃), —CH₂C—CH, —CH₂C═C(CH₃), and —CH₂C═C(CH₂CH₃) among others.

The term “amine” as used herein refers to primary, secondary, and tertiary amines having, e.g., the formula N (group)₃ wherein each group can independently be H or non-H, such as alkyl, aryl, and the like. Amines include but are not limited to R—NH₂, for example, alkylamines, arylamines, alkylarylamines: R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7.

Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cycloalkylene” or “cycloalkylenyl” as used herein refers to a bivalent saturated cycloalkyl radical

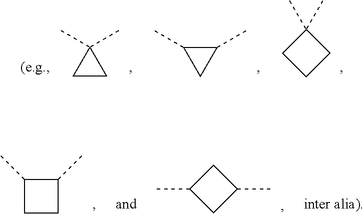

In certain embodiments, the term may be regarded as a product of removal of two hydrogen atoms from the corresponding cycloalkane (e.g. cyclobutyl) by removal of two hydrogen atoms from the same

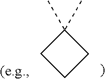

different

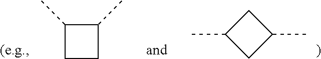

carbon atoms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “ameliorated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to. N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heteroarylene” or “heteroarylenyl” as used herein refers to a bivalent heteroaryl radical (e.g., 2,4-pyridylene). In certain embodiments, the term may be regarded as a divalent radical formed by the removal of two hydrogen atoms from one or more rings of a heteroaryl moiety, wherein the hydrogen atoms may be removed from the same or different rings, preferably the same ring.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom can be optionally substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The term heterocycloalkyl group can also be a C₂ heterocycloalkyl. C₂-C₃ heterocycloalkyl, C₂-C₄ heterocycloalkyl, C₂-C₅ heterocycloalkyl, C₂-C₆ heterocycloalkyl, C₂-C₇ heterocycloalkyl, C₂-C₈ heterocycloalkyl, C₂-C₉ heterocycloalkyl, C₂-C₁₀ heterocycloalkyl, C₂-C₁₁ heterocycloalkyl, and the like, up to and including a C₂-14₅ heterocycloalkyl. For example, a C₂ heterocycloalkyl comprises a group which has two carbon atoms and at least one heteroatom, including, but not limited to, aziridinyl, diazetidinyl, oxiranyl, thiiranyl, and the like. Alternatively, for example, a C₅ heterocycloalkyl comprises a group which has five carbon atoms and at least one heteroatom, including, but not limited to, piperidinyl, tetrahydropyranyl, tetrahydrothiopyranyl, diazepanyl, and the like. It is understood that a heterocycloalkyl group may be bound either through a heteroatom in the ring, where chemically possible, or one of carbons comprising the heterocycloalkyl ring. The heterocycloalkyl group can be substituted or unsubstituted.

The term “heterocycloalkylene” or “heterocycloalkylenyl” as used herein refers to a bivalent saturated cycloalkyl radical

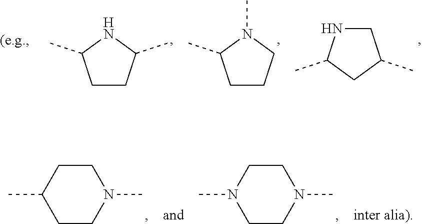

In certain embodiments, the term may be regarded as a product of removal of two hydrogen atoms from the corresponding heterocycloalkane (e.g. piperidine) by removal of two hydrogen atoms from the same

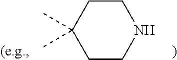

different

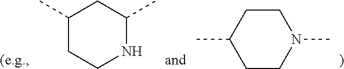

carbon atom(s) and/or heteroatom(s).

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “linker” as used herein refers to an organic moiety that connects two parts of a compound (e.g., two small molecule drugs, or a small molecule drug and an antibody). The linker can be, in non-limiting examples, a direct bond, a single atom (e.g., —O—), a peptide, or a substituted or unsubstituted alkylene or heteroalkylene moiety (e.g., polyethylene glycol). One skilled in the art would be apprised of the common linkers suitable for use in antibody drug conjugates and methods of preparation thereof.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group: a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient.” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate: powdered tragacanth: malt; gelatin; talc: excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol: esters, such as ethyl oleate and ethyl laurate; agar; buffering agents, such as magnesium hydroxide and aluminum hydroxide: surface active agents; alginic acid; pyrogen-free water; isotonic saline; Ringer's solution: ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro. Ed., Mack Publishing Co., 1985. Easton, PA), which is incorporated herein by reference.

The term “phenylene” or “phenylenyl” as used herein refers to a bivalent phenyl radical (e.g., 1,4-phenylene). In certain embodiments, the term may be regarded as a divalent radical formed by the removal of two hydrogen atoms from a benzene moiety.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “specifically binds”, or “specifically binds”, or the like, means that a small molecule, an antibody, and/or antigen-binding fragment forms a complex with a target and/or an antigen that is relatively stable under physiological conditions. The specific bond can be characterized by an equilibrium dissociation constant (for example, a smaller K_{D} denotes a firmer bond). Methods for determining whether two molecules specifically bind to each other are well known in the art and include, for example, equilibrium dialysis, surface plasmon resonance, and the like.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety: for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The terms “treat,” “treating” and “treatment,” as used herein, means reducing the frequency or severity with which symptoms of a disease or condition are experienced by a subject by virtue of administering an agent or compound to the subject.

### Compounds and Compositions

In one aspect, the present disclosure provides a compound of Formula (I), or a salt, solvate, stereoisomer, or isotopologue thereof:

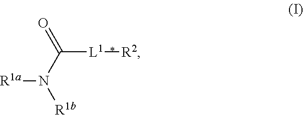

wherein:
R^{1a} is selected from the group consisting of C₁-C₆ alkyl, phenyl, and C₂-C₁₀ heteroaryl, wherein the alkyl, phenyl, or heteroaryl is optionally substituted with at least one substituent selected from the group consisting of optionally substituted C₁-C₆ alkyl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B};
R^{1b} is selected from the group consisting of H and optionally substituted C₁-C₆ alkyl, or
   R^{1a} and R^{1b} can combine with the nitrogen atom to which they are bound to form an optionally substituted C₂-C₈ heterocycloalkyl;
R² is selected from the group consisting of optionally substituted C₂-C₉ heteroaryl and optionally substituted C₆-C₁₀ aryl;
L¹ is selected from the group consisting of

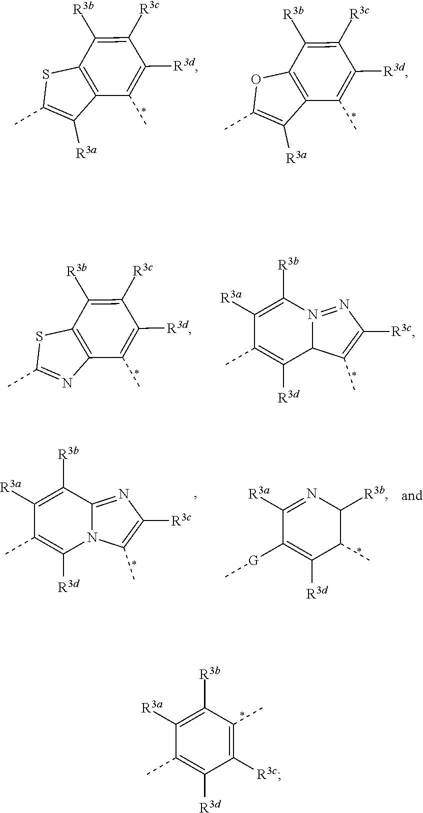

R^{3a}, R^{3b}, R^{3c}, and R^{3d} are each independently selected from the group consisting of H, optionally substituted C₁-C₆ alkyl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D}; and
Y is selected from the group consisting of optionally substituted C₁-C₃ alkylenyl and optionally substituted C₃-C₈ cycloalkylenyl;
R^{A}, R^{B}, R^{C}, and R^{D}, if present, are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl,
   wherein R^{A} and R^{B} or R^{C} and R^{D} can combine with the nitrogen atom to which they are bound form an optionally substituted C₂-C₈ heterocycloalkyl, and
   wherein one or more of R^{A} and R^{B} can combine with L to form an optionally substituted C₄-C₈ heterocycloalkyl or C₄-C₆ heteroaryl.

In certain embodiments, Y is optionally substituted 1,1-cyclopropylene.

In certain embodiments, the compound of Formula (I) is a compound of Formula (Ia):

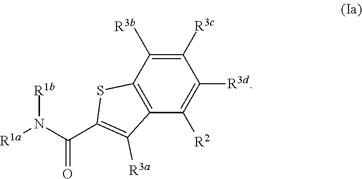

In certain embodiments, the compound of Formula (I) is a compound of Formula (Ib):

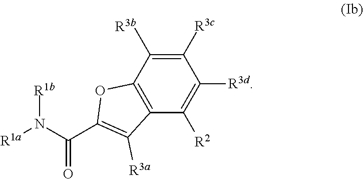

In certain embodiments, the compound of Formula (I) is a compound of Formula (Ic):

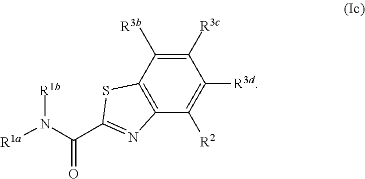

In certain embodiments, the compound of Formula (I) is a compound of Formula (Id):

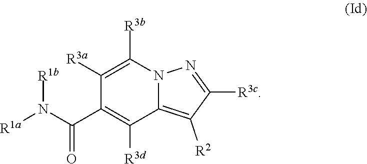

In certain embodiments, the compound of Formula (I) is a compound of Formula (Ie):

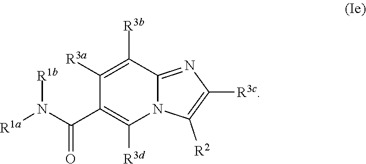

In certain embodiments, the compound of Formula (I) is a compound of Formula (If):

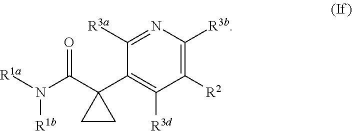

In certain embodiments, the compound of Formula (I) is a compound of Formula (Ig):

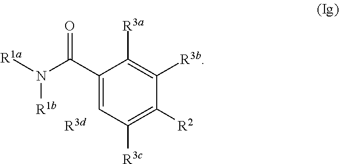

In certain embodiments, R^{1a} is phenyl substituted with at least one substituent selected from the group consisting of C₁-C₆ alkyl, C₁-C₆ haloalkyl, F, OH CN, O(C₁-C₆ alkyl), O(C₁-C₆ haloalkyl), —(CH₂)₁₋₃NH(C₁-C₆ alkyl), —(CH₂)₁₋₃N(C₁-C₆ alkyl)₂, C(═O)NH₂, C(═O)NH(C₁-C₆ alkyl), C(═O)N(C₁-C₆ alkyl)₂, and C(═O)(optionally substituted C₂-C₈ heterocycloalkyl).

In certain embodiments, R^{1a} is alkyl substituted with at least one substituent selected from the group consisting of C₁-C₆ alkyl, C₁-C₆ haloalkyl, F, OH. CN, O(C₁-C₆ alkyl), O(C₁-C₆ haloalkyl), —(CH₂)₁₋₃NH(C₁-C₆ alkyl), —(CH₂)₁₋₃N(C₁-C₆ alkyl)₂, C(═O)NH₂, C(═O)NH(C₁-C₆ alkyl), C(═O)N(C₁-C₆ alkyl)₂, and C(═O)(optionally substituted C₂-C₈ heterocycloalkyl).

In certain embodiments, R^{1a} is pyridinyl substituted with at least one substituent selected from the group consisting of C₁-C₆ alkyl, C₁-C₆ haloalkyl, F, OH, CN, O(C₁-C₆ alkyl), O(C₁-C₆ haloalkyl), —(CH₂)₁₋₃NH(C₁-C₆ alkyl). —(CH₂)₁₋₃N(C₁-C₆ alkyl)₂, C(═O)NH₂, C(═O)NH(C₁-C₆ alkyl), C(═O)N(C₁-C₆ alkyl)₂, and C(═O)(optionally substituted C₂-C₈ heterocycloalkyl).

In certain embodiments, R^{1a} is pyrimidinyl substituted with at least one substituent selected from the group consisting of C₁-C₆ alkyl, C₁-C₆ haloalkyl, F, OH, CN, O(C₁-C₆ alkyl), O(C₁-C₆ haloalkyl), —(CH₂)₁₋₃NH(C₁-C₆ alkyl). —(CH₂)₁₋₃N(C₁-C₆ alkyl)₂, C(═O)NH₂, C(═O)NH(C₁-C₆ alkyl), C(═O)N(C₁-C₆ alkyl)₂, and C(═O)(optionally substituted C₂-C₈ heterocycloalkyl).

In certain embodiments, R^{1a} is phenyl substituted with CH₃. In certain embodiments, R^{1a} is phenyl substituted with OH. In certain embodiments, R^{1a} is phenyl substituted with OCH₃. In certain embodiments. R^{1a} is phenyl substituted with OCF₃. In certain embodiments, R^{1a} is phenyl substituted with F. In certain embodiments, R^{1a} is phenyl substituted with CN.

In certain embodiments, R^{1a} is phenyl substituted with CH₂NHCH₃. In certain embodiments, R^{1a} is phenyl substituted with CH₂N(CH₃)₂. In certain embodiments, R^{1a} is phenyl substituted with C(═O)NH₂. In certain embodiments, R^{1a} is phenyl substituted with C(═O)NHCH₃. In certain embodiments, R^{1a} is phenyl substituted with C(═O)N(CH₃)₂. In certain embodiments, R^{1a} is phenyl substituted with C(═O)(azetidinyl). In certain embodiments, R^{1a} is phenyl substituted with C(═O)(piperidinyl). In certain embodiments, R^{1a} is phenyl substituted with C(═O)(4-methylpiperazinyl). In certain embodiments. R^{1a} is phenyl substituted with C(═O)(morpholinyl). In certain embodiments, R^{1a} is phenyl substituted with C(═O)NH(oxiranyl). In certain embodiments, R^{1a} is phenyl substituted with C(═O)NH(cyclopropylmethyl).

In certain embodiments, R^{1a} is pyridinyl substituted with CH₃. In certain embodiments, R^{1a} is pyridinyl substituted with OH. In certain embodiments, R^{1a} is pyridinyl substituted with OCH₃. In certain embodiments, R^{1a} is pyridinyl substituted with OCF₃. In certain embodiments, R^{1a} is pyridinyl substituted with F. In certain embodiments, R^{1a} is pyridinyl substituted with CN. In certain embodiments, R^{1a} is pyridinyl substituted with CH₂NHCH₃. In certain embodiments, R^{1a} is pyridinyl substituted with CH₂N(CH₃)₂. In certain embodiments, R^{1a} is pyridinyl substituted with C(═O)NH₂. In certain embodiments, R^{1a} is pyridinyl substituted with C(═O)NHCH₃. In certain embodiments, R^{1a} is pyridinyl substituted with C(═O)N(CH₃)₂. In certain embodiments, R^{1a} is pyridinyl substituted with C(═O)(azetidinyl). In certain embodiments, R^{1a} is pyridinyl substituted with C(═O)(piperidinyl). In certain embodiments, R^{1a} is pyridinyl substituted with C(═O)(4-methylpiperazinyl). In certain embodiments, R^{1a} is pyridinyl substituted with C(═O)(morpholinyl). In certain embodiments, R^{1a} is pyridinyl substituted with C(═O)NH(oxiranyl). In certain embodiments, R^{1a} is pyridinyl substituted with C(═O)NH(cyclopropylmethyl).

In certain embodiments, R^{1a} is pyrimidinyl substituted with CH₃. In certain embodiments, R^{1a} is pyrimidinyl substituted with OH. In certain embodiments, R^{1a} is pyrimidinyl substituted with OCH₃. In certain embodiments, R^{1a} is pyrimidinyl substituted with OCF₃. In certain embodiments. R^{1a} is pyrimidinyl substituted with F. In certain embodiments. R^{1a} is pyrimidinyl substituted with CN. In certain embodiments, R^{1a} is pyrimidinyl substituted with CH₂NHCH₃. In certain embodiments, R^{1a} is pyrimidinyl substituted with CH₂N(CH₃)₂. In certain embodiments, R^{1a} is pyrimidinyl substituted with C(═O)NH₂. In certain embodiments, R^{1a} is pyrimidinyl substituted with C(═O)NHCH₃. In certain embodiments, R^{1a} is pyrimidinyl substituted with C(═O)N(CH₃)₂. In certain embodiments, R^{1a} is pyrimidinyl substituted with C(═O)(azetidinyl). In certain embodiments, R^{1a} is pyrimidinyl substituted with C(═O)(piperidinyl). In certain embodiments, R^{1a} is pyrimidinyl substituted with C(═O)(4-methylpiperazinyl). In certain embodiments. R^{1a} is pyrimidinyl substituted with C(═O)(morpholinyl). In certain embodiments, R^{1a} is pyrimidinyl substituted with C(═O)NH(oxiranyl). In certain embodiments, R^{1a} is pyrimidinyl substituted with C(═O)NH(cyclopropylmethyl).

In certain embodiments, R^{1a} is

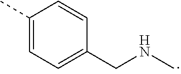

In certain embodiments, R^{1a} is

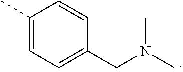

In certain embodiments, R^{1a} is

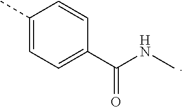

In certain embodiments, R^{1a} is

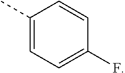

In certain embodiments, R^{1a} is

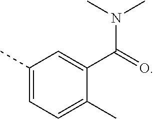

In certain embodiments, R^{1a} is

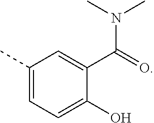

In certain embodiments, R^{1a} is

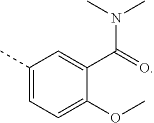

In certain embodiments, R^{1a} is

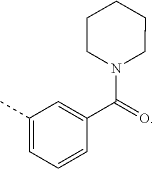

In certain embodiments, R^{1a} is

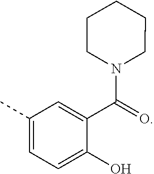

In certain embodiments, R¹ is

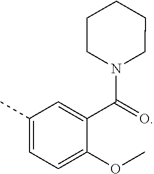

In certain embodiments, R^{1a} is

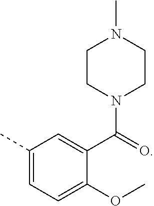

In certain embodiments, R^{1a} is

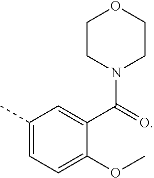

In certain embodiments, R^{1a} is

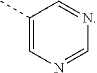

In certain embodiments, R^{1a} is

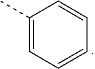

In certain embodiments, R^{1a} is

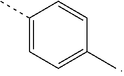

In certain embodiments, R^{1a} is

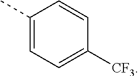

In certain embodiments, R^{1a} is

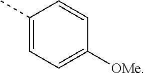

In certain embodiments, R^{1a} is

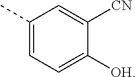

In certain embodiments, R^{1a} is

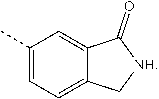

In certain embodiments, R^{1a} is

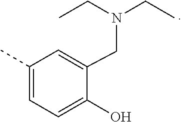

In certain embodiments, R^{1a} is

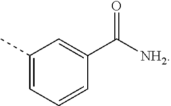

In certain embodiments, R^{1a} is

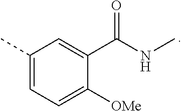

In certain embodiments, R^{1a} is

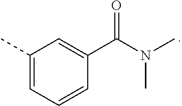

In certain embodiments, R^{1a} is

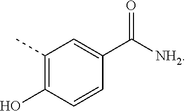

In certain embodiments, R^{1a} is

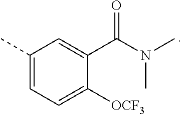

In certain embodiments, R^{1a} is

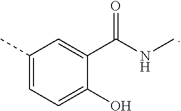

In certain embodiments, R^{1a} is

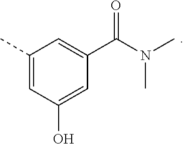

In certain embodiments, R^{1a} is

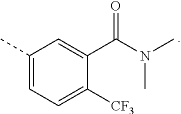

In certain embodiments, R^{1a} is

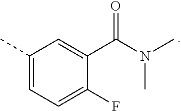

In certain embodiments, R^{1a} is

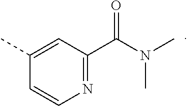

In certain embodiments, R^{1a} is

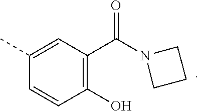

In certain embodiments, R^{1a} is

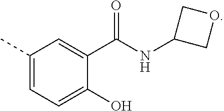

In certain embodiments, R^{1a} is

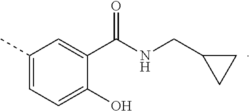

In certain embodiments, R^{1a} is optionally substituted C₁-C₆ alkyl.

In certain embodiments, R^{1a} is methyl. In certain embodiments, R^{1a} is CH₂CH₂N(CH₃)₂. In certain embodiments, R^{1b} is H.

In certain embodiments, R^{1a} and R^{1b} combine with the nitrogen atom to which they are bound to form

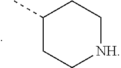

In certain embodiments, R^{1a} and R^{1b} combine with the nitrogen atom to which they are bound to form

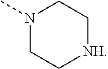

In certain embodiments, R^{1a} and R^{1b} combine with the nitrogen atom to which they are bound to form

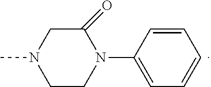

In certain embodiments, R² is

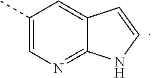

In certain embodiments, R² is

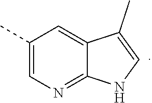

In certain embodiments, R² is

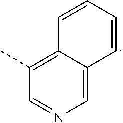

In certain embodiments, R² is

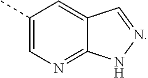

In certain embodiments, R² is

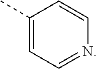

In certain embodiments, R² is

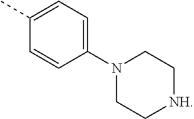

In certain embodiments, R² is

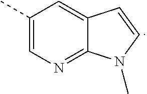

In certain embodiments, at least one of R^{3a}, R^{3b}, R^{3c}, and R^{3d} is H. In certain embodiments, at least two of R^{3a}, R^{3b}, R^{3c}, and R^{3d} are H. In certain embodiments, at least three of R^{3a}, R^{3b}, R^{3c}, and R^{3d} are H. In certain embodiments, each of R^{3a}, R^{3b}, R^{3c}, and R^{3d} are H.

In certain embodiments, the compound is selected from the group consisting of:
N-methyl-3-(1H-pyrrolo[2,3-b]pyridin-5-yl)pyrazolo[1,5-a]pyridine-5-carboxamide;
3-(isoquinolin-4-yl)-N-(p-tolyl)pyrazolo[1,5-a]pyridine-5-carboxamide;
N-(4-((methylamino)methyl)phenyl)-3-(1H-pyrrolo[2,3-b]pyridin-5-yl)pyrazolo[1,5-a]pyridine-5-carboxamide;
3-(isoquinolin-4-yl)-N-(4-((methylamino)methyl)phenyl)pyrazolo[1,5-a]pyridine-5-carboxamide;
N-(4-((methylamino)methyl)phenyl)-3-(1H-pyrrolo[2,3-b]pyridin-5-yl)imidazo[1,2-a]pyridine-6-carboxamide;
N-(4-((methylamino)methyl)phenyl)-4-(1H-pyrrolo[2,3-b]pyridin-5-yl)benzo[b]thiophene-2-carboxamide;
N-(3-(dimethylcarbamoyl)-4-methoxyphenyl)-4-(1H-pyrrolo[2,3-b]pyridin-5-yl)benzo[b]thiophene-2-carboxamide;
N-(4-methoxy-3-(piperidine-1-carbonyl)phenyl)-4-(1H-pyrrolo[2,3-b]pyridin-5-yl)benzo[b]thiophene-2-carboxamide;
3-(2-aminopyrimidin-5-yl)-N-(4-((methylamino)methyl)phenyl)pyrazolo[1,5-a]pyridine-5-carboxamide;
N-(4-((dimethylamino)methyl)phenyl)-3-(1H-pyrrolo[2,3-b]pyridin-5-yl)pyrazolo[1,5-a]pyridine-5-carboxamide;
N-(4-(methylcarbamoyl)phenyl)-3-(1H-pyrrolo[2,3-b]pyridin-5-yl)pyrazolo[1,5-a]pyridine-5-carboxamide;
N-(4-fluorophenyl)-3-(1H-pyrrolo[2,3-b]pyridin-5-yl)pyrazolo[1,5-a]pyridine-5-carboxamide;
N-(4-methoxy-3-(morpholine-4-carbonyl)phenyl)-4-(1H-pyrrolo[2,3-b]pyridin-5-yl)benzo[b]thiophene-2-carboxamide;
N-(4-methoxy-3-(4-methylpiperazine-1-carbonyl)phenyl)-4-(1H-pyrrolo[2,3-b]pyridin-5-yl)benzo[b]thiophene-2-carboxamide;
N-(3-(piperidine-1-carbonyl)phenyl)-4-(1H-pyrrolo[2,3-b]pyridin-5-yl)benzo[b]thiophene-2-carboxamide;
N-(4-hydroxy-3-(piperidine-1-carbonyl)phenyl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzo[b]thiophene-2-carboxamide;
N-(3-(dimethylcarbamoyl)-4-methylphenyl)-4-(1H-pyrrolo[2,3-b]pyridin-5-yl)benzo[b]thiophene-2-carboxamide;
N-(3-(dimethylcarbamoyl)-4-methoxyphenyl)-4-(3-methyl-1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzofuran-2-carboxamide;
N-(3-(dimethylcarbamoyl)-4-hydroxyphenyl)-4-(1H-pyrrolo[2,3-b]pyridin-5-yl)benzo[d]thiazole-2-carboxamide;
N-(3-(dimethylcarbarnoyl)-4-(trifluoromethoxy)phenyl)-4-(1H-pyrrolo[2,3-b]pyridin-5-yl)benzo[b]thiophene-2-carboxamide;
N-(3-carbamoylphenyl)-4-(1H-pyrrolo[2,3-b]pyridin-5-yl)benzo[b]thiophene-2-carboxamide;
N-(3-(dimethylcarbamoyl)-5-hydroxyphenyl)-4-(1H-pyrrolo[2,3-b]pyridin-5-yl)benzo[b]thiophene-2-carboxamide;
N-(5-(dimethylcarbamoyl)-2-hydroxyphenyl)-4-(1H-pyrrolo[2,3-b]pyridin-5-yl)benzo[b]thiophene-2-carboxamide;
4-(4-(1H-pyrrolo[2,3-b]pyridin-5-yl)benzo[b]thiophene-2-carboxamido)-N,N-dimethylpicolinamide:
N-(4-hydroxy-3-(pyrrolidine-1-carbonyl)phenyl)-4-(1H-pyrrolo[2,3-b]pyridin-5-yl)benzo[b]thiophene-2-carboxamide;
N-(3-(azetidine-1-carbonyl)-4-hydroxyphenyl)-4-(1H-pyrrolo[2,3-b]pyridin-5-yl)benzo[b]thiophene-2-carboxamide;
N-(pyrimidin-5-yl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzo[d]thiazole-2-carboxamide;
N-(3-(dimethylcarbamoyl)-4-hydroxyphenyl)-4-(3-methyl-1H-pyrrolo[2,3-b]pyridin-5-yl)benzo[b]thiophene-2-carboxamide;
N-(3-(dimethylcarbamoyl)-4-hydroxyphenyl)-4-(1H-pyrazolo[3,4-b]pyridin-5-yl)benzo[b]thiophene-2-carboxamide;
N-(2-(dimethylamino)ethyl)-4-(1H-pyrrolo[2,3-b]pyridin-5-yl)benzo[b]thiophene-2-carboxamide;
N-(3-((2-(dimethylamino)ethyl)carbamoyl)-4-hydroxyphenyl)-4-(1H-pyrrolo[2,3-b]pyridin-5-yl)benzo[b]thiophene-2-carboxamide;
N-(4-hydroxy-3-(methylcarbamoyl)phenyl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)thieno[2,3-c]pyridine-2-carboxamide;
N-(4-hydroxy-3-(oxetan-3-ylcarbonyl)phenyl)-4-(1H-pyrrolo[2,3-b]pyridin-5-yl)benzo[b]thiophene-2-carboxamide;
N-(3-((cyclopropylmethyl)carbamoyl)-4-hydroxyphenyl)-4-(1H-pyrrolo[2,3-b]pyridin-5-yl)benzo[b]thiophene-2-carboxamide;
N-(3-oxoisoindolin-5-yl)-4-(1H-pyrrolo[2,3-b]pyridin-5-yl)benzo[b]thiophene-2-carboxamide;
4-(4-(1H-pyrrolo[2,3-b]pyridin-5-yl)benzo[b]thiophene-2-carbonyl)-1-phenylpiperazin-2-one;
N-(3-((diethylamino)methyl)-4-hydroxyphenyl)-4-(1H-pyrrolo[2,3-b]pyridin-5-yl)benzo[b]thiophene-2-carboxamide;
N-(3-cyano-4-hydroxyphenyl)-4-(1H-pyrrolo[2,3-b]pyridin-5-yl)benzo[b]thiophene-2-carboxamide;
N-phenyl-3-(1H-pyrrolo[2,3-b]pyridin-5-yl)pyrazolo[1,5-a]pyridine-5-carboxamide;
3-(1H-pyrrolo[2,3-b]pyridin-5-yl)-N-(p-tolyl)pyrazolo[1,5-a]pyridine-5-carboxamide;
3-(pyridin-4-yl)-N-(p-tolyl)pyrazolo[1,5-a]pyridine-5-carboxamide;
3-(1H-indol-5-yl)-N-(p-tolyl)pyrazolo[1,5-a]pyridine-5-carboxamide;
3-(1H-indazol-5-yl)-N-(4-((methylamino)methyl)phenyl)pyrazolo[1,5-a]pyridine-5-carboxamide;
N-(4-((methylamino)methyl)phenyl)-3-(4-(piperazin-1-yl)phenyl)pyrazolo[1,5-a]pyridine-5-carboxamide;
3-(1-methyl-1H-pyrrolo[2,3-b]pyridin-5-yl)-N-(4-((methylamino)methyl)phenyl)pyrazolo[1,5-a]pyridine-5-carboxamide;
3-(1H-pyrrolo[2,3-b]pyridin-5-yl)-N-(4-(trifluoromethyl)phenyl)pyrazolo[1,5-a]pyridine-5-carboxamide;
(3-(1H-pyrrolo[2,3-b]pyridin-5-yl)pyrazolo[1,5-a]pyridin-5-yl)(piperidin-1-yl)methanone;
(3-(1H-pyrrolo[2,3-b]pyridin-5-yl)pyrazolo[1,5-a]pyridin-5-yl)(piperazin-1-yl)methanone;
1-(5-(1H-pyrrolo[2,3-b]pyridin-5-yl)pyridin-3-yl)-N-(4 ((methylamino)methyl)phenyl)cyclopropane-1-carboxamide;
N-(4-((methylamino)methyl)phenyl)-4-(1H-pyrrolo[2,3-b]pyridin-5-yl)benzamide;
N-methyl-4-(1H-pyrrolo[2,3-b]pyridin-5-yl)benzo[b]thiophene-2-carboxamide;
N-phenyl-4-(1H-pyrrolo[2,3-b]pyridin-5-yl)benzo[b]thiophene-2-carboxamide;
N-(4-methoxyphenyl)-4-(1H-pyrrolo[2,3-b]pyridin-5-yl)benzo[b]thiophene-2-carboxamide;
N-(4-methoxy-3-(methylcarbamoyl)phenyl)-4-(1H-pyrrolo[2,3-b]pyridin-5-yl)benzo[b]thiophene-2-carboxamide;
N-(4-methoxy-3-(4-methylpiperazine-1-carbonyl)phenyl)-4-(1H-pyrrolo[2,3-b]pyridin-5-yl)benzo[b]thiophene-2-carboxamide;
N-(3-(dimethylcarbamoyl)phenyl)-4-(1H-pyrrolo[2,3-b]pyridin-5-yl)benzo[b]thiophene-2-carboxamide;
N-(3-(dimethylcarbamoyl)-4-(trifluoromethyl)phenyl)-4-(1H-pyrrolo[2,3-b]pyridin-5-yl)benzo[b]thiophene-2-carboxamide; and
N-(3-(dimethylcarbamoyl)-4-fluorophenyl)-4-(1H-pyrrolo[2,3-b]pyridin-5-yl)benzo[b]thiophene-2-carboxamide.

In another aspect, the present disclosure provides a compound of Formula (II), or a salt, solvate, stereoisomer, or isotopologue thereof:

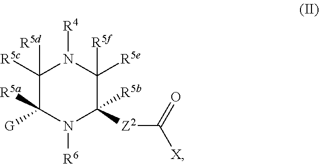

wherein:
R⁴ is selected from the group consisting of H, optionally substituted C₁-C₆ alkyl, optionally substituted C₃-C₈ cycloalkyl, optionally substituted C₂-C₆ heterocycloalkyl, optionally substituted C₇-C₁₂ aralkyl, optionally substituted C₃-C₁₂ heteroaralkyl, optionally substituted C₆-C₁₀ aryl, and optionally substituted C₂-C₁₂ heteroaryl;
R^{5a}, R^{5b}, R^{5c}, R^{5d}, R^{5e}, and R^{5f} are each independently selected from the group consisting of H, optionally substituted C₁-C₆ alkyl, halogen. OR^{E}, and N(R^{E})(R^{F});
R⁶ is —(CH₂)₁₋₃C(═O)N(R^{G})(R^{H});
R^{7a} is selected from the group consisting of H and C(═O)OR
R^{7b} is H;
X is selected from the group consisting of OR^{J} and

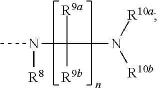

G is selected from the group consisting of optionally substituted C₁-C₆ alkyl and —Z¹—N(R^{7a})(R^{7b});
Z¹ and Z² are each independently-(optionally substituted C₁-C₆ alkylenyl)-;
R⁸ is selected from the group consisting of H and optionally substituted C₁-C₆ alkyl;
R^{9a} and R^{9b} are each independently selected from the group consisting of H and optionally substituted C₁-C₆ alkyl;
R^{10a} and R^{10b} are each independently selected from the group consisting of H, optionally substituted C₁-C₆ alkyl, and optionally substituted C₆-C₁₀ aryl;
n is 1, 2, 3, 4, or 5; and
R^{E}, R^{F}, R^{G}, R^{H}, R^{I} and R^{J} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

In certain embodiments, R⁴ is H. In certain embodiments, R⁴ is

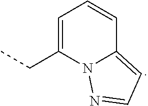

In certain embodiments, at least one of R^{5a}, R^{5b}, R^{5c}, R^{5d}, R^{5e}, and R^{5f} is H. In certain embodiments, at least two of R⁵, R^{5b}, R^{5c}, R^{5d}, R^{5e}, and R^{5f} are H. In certain embodiments, at least three of R^{5a}, R^{5b}, R^{5c}, R^{5d}, R^{5e} and R^{5f} are H. In certain embodiments, at least four of R^{5a}, R^{5b}, R^{5c}, R^{5d}, R^{5e}, and R^{5f} are H. In certain embodiments, at least five of R^{5a}, R^{5b}, R^{5c}, R^{5d}, R^{5e}, and R^{5f} are H. In certain embodiments, each of R^{5a}, R^{5b}, R^{5c}, R^{5d}, R^{5e}, and R^{5f} are H.

In certain embodiments, R⁶ is —CH₂C(═O)NHCH₃.

In certain embodiments, G is CH₃.

In certain embodiments, G is —Z¹—N(R^{7a})(R^{7b}).

In certain embodiments, R^{7a} is H. In certain embodiments, R^{7a} is

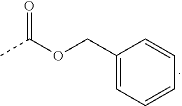

In certain embodiments, R^{7b} is H.

In certain embodiments, X is OEt. In certain embodiments, X is

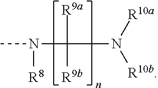

In certain embodiments, R^{9a} is H. In certain embodiments, R^{9b} is H.

In certain embodiments, n is 3.

In certain embodiments, R^{10a} is methyl. In certain embodiments, R^{10a} is 4-fluorophenyl.

In certain embodiments, R^{10b} is methyl. In certain embodiments, R^{10b} is 4-fluorophenyl.

In certain embodiments, X is

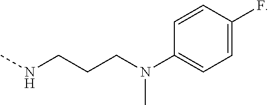

In certain embodiments, Z¹ is —CH₂CH₂CH₂—.

In certain embodiments, Z² is —CH₂—.

In certain embodiments, the compound is selected from the group consisting of
benzyl (2-((2R,6R)-6-(2-((3-((4-fluorophenyl)(methyl)amino)propyl)amino)-2-oxoethyl)-1-(2-(methylamino)-2-oxoethyl)-4-(pyrazolo[1,5-a]pyridin-7-ylmethyl)piperazin-2-yl)ethyl)carbamate;
ethyl 2-((2R,6R)-6-(2-(((benzyloxy)carbonyl)amino)ethyl)-1-(2-(methylamino)-2-oxoethyl)-4-(pyrazolo[1,5-a]pyridin-7-ylmethyl)piperazin-2-yl)acetate;
benzyl (2-((2R,6R)-6-(2-((3-((4-fluorophenyl)(methyl)amino)propyl)amino)-2-oxoethyl)-1-(2-(methylamino)-2-oxoethyl)piperazin-2-yl)ethyl)carbamate; and
N-(3-((4-fluorophenyl)(methyl)amino)propyl)-2-((2R,6R)-6-methyl-1-(2-(methylamino)-2-oxoethyl)piperazin-2-yl)acetamide.

TABLE 1
Exemplary compounds

| Cmpd | Structure | Nomenclature |
|---|---|---|
| 1 CDD- 2574 | 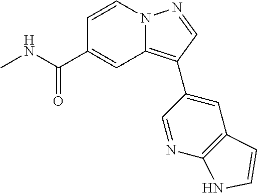 [SMILES: CNC(=O)c1ccn2ncc(-c3cnc4[nH]ccc4c3)c2c1] | N-methyl-3-(1H-pyrrolo[2,3-b]pyridin-5- yl)pyrazolo[1,5-a]pyridine-5-carboxamide |
| 2 CDD- 2325 | 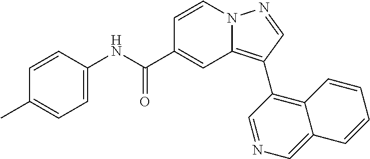 [SMILES: Cc1ccc(NC(=O)c2ccn3ncc(-c4cncc5ccccc45)c3c2)cc1] | 3-(isoquinolin-4-yl)-N-(p- tolyl)pyrazolo[1,5-a]pyridine-5- carboxamide |
| 3 CDD- 2346 | 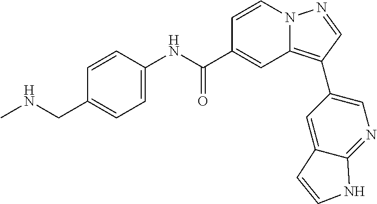 [SMILES: CNCc1ccc(NC(=O)c2ccn3ncc(-c4cnc5[nH]ccc5c4)c3c2)cc1] | N-(4-((methylamino)methyl)phenyl)-3-(1H- pyrrolo[2,3-b]pyridin-5-yl)pyrazolo[1,5- a]pyridine-5-carboxamide |
| 4 CDD- 2347 | 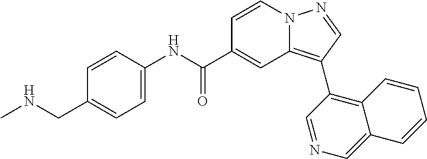 [SMILES: CNCc1ccc(NC(=O)c2ccn3ncc(-c4cncc5ccccc45)c3c2)cc1] | 3-(isoquinolin-4-yl)-N-(4- ((methylamino)methyl)phenyl)pyrazolo [1,5-a]pyridine-5-carboxamide |
| 5 CDD- 2488 | 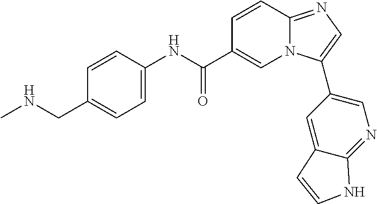 [SMILES: CNCc1ccc(NC(=O)c2ccc3ncc(-c4cnc5[nH]ccc5c4)n3c2)cc1] | N-(4-((methylamino)methyl)phenyl)-3-(1H- pyrrolo[2,3-b]pyridin-5-yl)imidazo[1,2- a]pyridine-6-carboxamide |
| 6 CDD- 2575 | 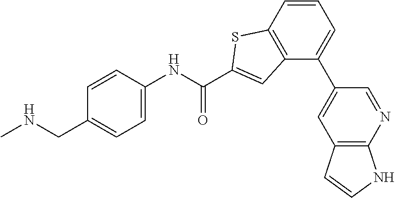 [SMILES: CNCc1ccc(NC(=O)c2cc3c(-c4cnc5[nH]ccc5c4)cccc3s2)cc1] | N-(4-((methylamino)methyl)phenyl)-4-(1H- pyrrolo[2,3-b]pyridin-5- yl)benzo[b]thiophene-2-carboxamide |
| 7 CDD- 2628 | 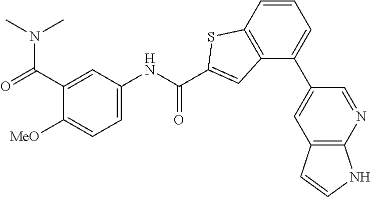 [SMILES: COc1ccc(NC(=O)c2cc3c(-c4cnc5[nH]ccc5c4)cccc3s2)cc1C(=O)N(C)C] | N-(3-(dimethylcarbamoyl)-4- methoxyphenyl)-4-(1H-pyrrolo[2,3- b]pyridin-5-yl)benzo[b]thiophene-2- carboxamide |
| 8 CDD- 2491 | 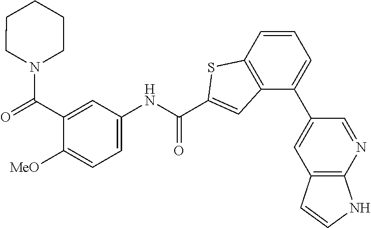 [SMILES: COc1ccc(NC(=O)c2cc3c(-c4cnc5[nH]ccc5c4)cccc3s2)cc1C(=O)N1CCCCC1] | N-(4-methoxy-3-(piperidine-1- carbonyl)phenyl)-4-(1H-pyrrolo[2,3- b]pyridin-5-yl)benzo[b]thiophene-2- carboxamide |
| 9 CDD- 2387 | 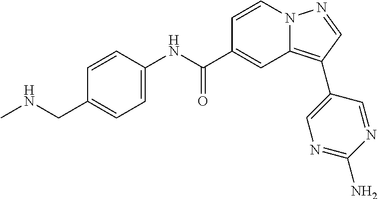 [SMILES: CNCc1ccc(NC(=O)c2ccn3ncc(-c4cnc(N)nc4)c3c2)cc1] | 3-(2-aminopyrimidin-5-yl)-N-(4- ((methylamino)methyl)phenyl)pyrazolo [1,5-a]pyridine-5-carboxamide |
| 10 CDD- 2482 | 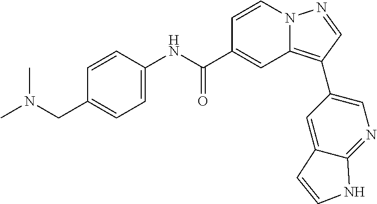 [SMILES: CN(C)Cc1ccc(NC(=O)c2ccn3ncc(-c4cnc5[nH]ccc5c4)c3c2)cc1] | N-(4-((dimethylamino)methyl)phenyl)-3- (1H-pyrrolo[2,3-b]pyridin-5- yl)pyrazolo[1,5-a]pyridine-5-carboxamide |
| 11 CDD- 2461 | 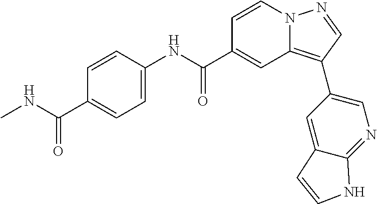 [SMILES: CNC(=O)c1ccc(NC(=O)c2ccn3ncc(-c4cnc5[nH]ccc5c4)c3c2)cc1] | N-(4-(methylcarbamoyl)phenyl)-3-(1H- pyrrolo[2,3-b]pyridin-5-yl)pyrazolo[1,5- a]pyridine-5-carboxamide |
| 12 CDD- 2486 | 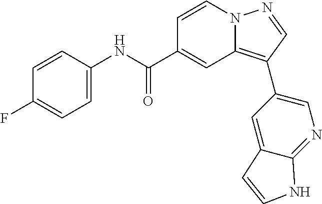 [SMILES: O=C(Nc1ccc(F)cc1)c1ccn2ncc(-c3cnc4[nH]ccc4c3)c2c1] | N-(4-fluorophenyl)-3-(1H-pyrrolo[2,3- b]pyridin-5-yl)pyrazolo[1,5-a]pyridine-5- carboxamide |
| 13 CDD- 2635 | 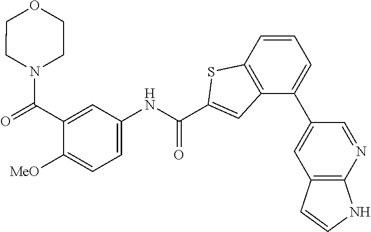 [SMILES: COc1ccc(NC(=O)c2cc3c(-c4cnc5[nH]ccc5c4)cccc3s2)cc1C(=O)N1CCOCC1] | N-(4-methoxy-3-(morpholine-4- carbonyl)phenyl)-4-(1H-pyrrolo[2,3- b]pyridin-5-yl)benzo[b]thiophene-2- carboxamide |
| 14 CD- 2636 | 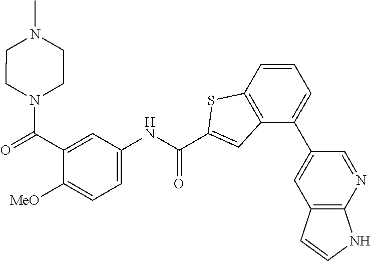 [SMILES: COc1ccc(NC(=O)c2cc3c(-c4cnc5[nH]ccc5c4)cccc3s2)cc1C(=O)N1CCN(C)CC1] | N-(4-methoxy-3-(4-methylpiperazine-1- carbonyl)phenyl)-4-(1H-pyrrolo[2,3- b]pyridin-5-yl)benzo[b]thiophene-2- carboxamide |
| 15 CDD- 2630 | 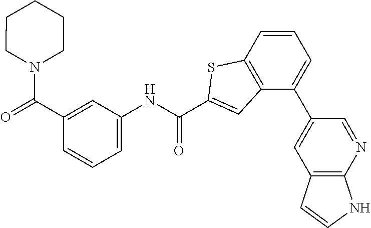 [SMILES: O=C(Nc1cccc(C(=O)N2CCCCC2)c1)c1cc2c(-c3cnc4[nH]ccc4c3)cccc2s1] | N-(3-(piperidine-1-carbonyl)phenyl)-4-(1H- pyrrolo[2,3-b]pyridin-5- yl)benzo[b]thiophene-2-carboxamide |
| 16 CDD- 2634 | 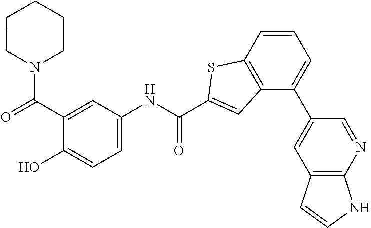 [SMILES: O=C(Nc1ccc(O)c(C(=O)N2CCCCC2)c1)c1cc2c(-c3cnc4[nH]ccc4c3)cccc2s1] | N-(4-hydroxy-3-(piperidine-1- carbonyl)phenyl)-4-(1H-pyrrolo[2,3- b]pyridin-5-yl)benzo[b]thiophene-2- carboxamide |
| 17 CDD- 2728 | 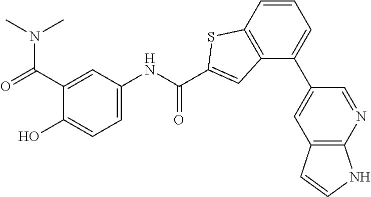 [SMILES: CN(C)C(=O)c1cc(NC(=O)c2cc3c(-c4cnc5[nH]ccc5c4)cccc3s2)ccc1O] | N-(3-(dimethylcarbamoyl)-4- hydroxyphenyl)-4-(1H-pyrrolo[2,3- b]pyridin-5-yl)benzo[b]thiophene-2- carboxamide |
| 18 CDD- 2729 | 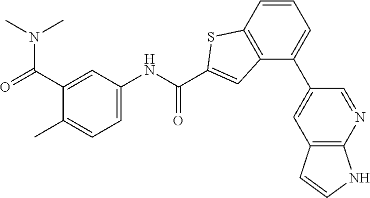 [SMILES: Cc1ccc(NC(=O)c2cc3c(-c4cnc5[nH]ccc5c4)cccc3s2)cc1C(=O)N(C)C] | N-(3-(dimethylcarbamoyl)-4- methylphenyl)-4-(1H-pyrrolo[2,3- b]pyridin-5-yl)benzo[b]thiophene-2- carboxamide |
| 19 CDD- 2731 | 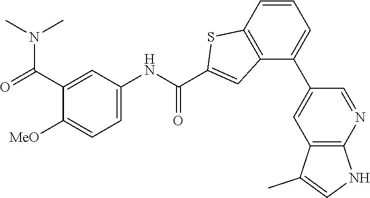 [SMILES: COc1ccc(NC(=O)c2cc3c(-c4cnc5[nH]cc(C)c5c4)cccc3s2)cc1C(=O)N(C)C] | N-(3-(dimethylcarbamoyl)-4- methoxyphenyl)-4-(3-methyl-1H- pyrrolo[2,3-b]pyridin-5- yl)benzo[b]thiophene-2-carboxamide |
| 20 CDD- 2855 | 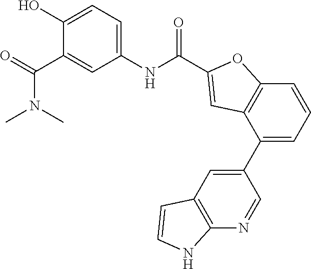 [SMILES: CN(C)C(=O)c1cc(NC(=O)c2cc3c(-c4cnc5[nH]ccc5c4)cccc3o2)ccc1O] | N-(3-(dimethylcarbamoyl)-4- hydroxyphenyl)-4-(1H-pyrrolo[2,3- b]pyridin-5-yl)benzofuran-2-carboxamide |
| 21 CDD- 2856 | 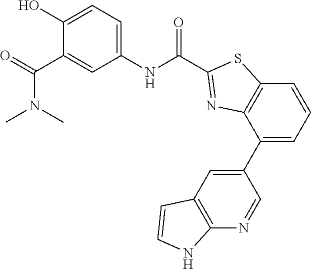 [SMILES: CN(C)C(=O)c1cc(NC(=O)c2nc3c(-c4cnc5[nH]ccc5c4)cccc3s2)ccc1O] | N-(3-(dimethylcarbamoyl)-4- hydroxyphenyl)-4-(1H-pyrrolo[2,3- b]pyridin-5-yl)benzo[d]thiazole-2- carboxamide |
| 22 CDD- 2857 | 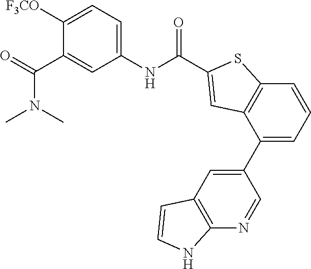 [SMILES: CN(C)C(=O)c1cc(NC(=O)c2cc3c(-c4cnc5[nH]ccc5c4)cccc3s2)ccc1OC(F)(F)F] | N-(3-(dimethylcarbamoyl)-4- (trifluoromethoxy)phenyl)-4-(1H- pyrrolo[2,3-b]pyridin-5- yl)benzo[b]thiophene-2-carboxamide |
| 23 CDD- 2936 | 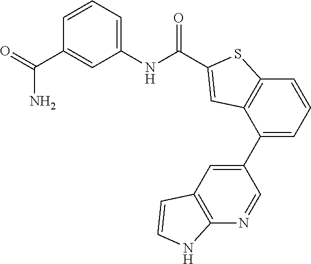 [SMILES: NC(=O)c1cccc(NC(=O)c2cc3c(-c4cnc5[nH]ccc5c4)cccc3s2)c1] | N-(3-carbamoylphenyl)-4-(1H-pyrrolo[2,3- b]pyridin-5-yl)benzo[b]thiophene-2- carboxamide |
| 24 CDD- 2937 | 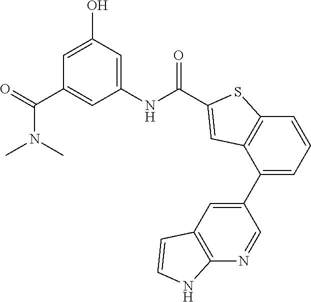 [SMILES: CN(C)C(=O)c1cc(O)cc(NC(=O)c2cc3c(-c4cnc5[nH]ccc5c4)cccc3s2)c1] | N-(3-(dimethylcarbamoyl)-5- hydroxyphenyl)-4-(1H-pyrrolo[2,3- b]pyridin-5-yl)benzo[b]thiophene-2- carboxamide |
| 25 CDD- 2938 | 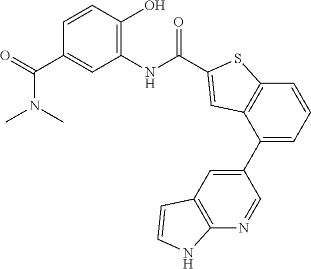 [SMILES: CN(C)C(=O)c1ccc(O)c(NC(=O)c2cc3c(-c4cnc5[nH]ccc5c4)cccc3s2)c1] | N-(5-(dimethylcarbamoyl)-2- hydroxyphenyl)-4-(1H-pyrrolo[2,3- b]pyridin-5-yl)benzo[b]thiophene-2- carboxamide |
| 26 CDD- 3012 | 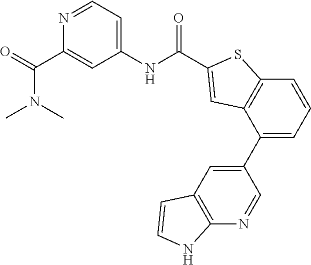 [SMILES: CN(C)C(=O)c1cc(NC(=O)c2cc3c(-c4cnc5[nH]ccc5c4)cccc3s2)ccn1] | 4-(4-(1H-pyrrolo[2,3-b]pyridin-5- yl)benzo[b]thiophene-2-carboxamido)-N,N- dimethylpicolinamide |
| 27 CDD- 3013 | 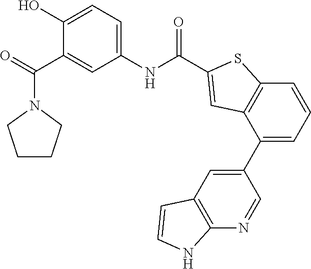 [SMILES: O=C(Nc1ccc(O)c(C(=O)N2CCCC2)c1)c1cc2c(-c3cnc4[nH]ccc4c3)cccc2s1] | N-(4-hydroxy-3-(pyrrolidine-1- carbonyl)phenyl)-4-(1H-pyrrolo[2,3- b]pyridin-5-yl)benzo[b]thiophene-2- carboxamide |
| 28 CDD- 3014 | 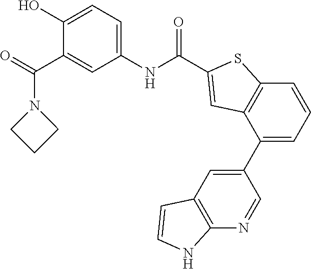 [SMILES: O=C(Nc1ccc(O)c(C(=O)N2CCC2)c1)c1cc2c(-c3cnc4[nH]ccc4c3)cccc2s1] | N-(3-(azetidine-1-carbonyl)-4- hydroxyphenyl)-4-(1H-pyrrolo[2,3- b]pyridin-5-yl)benzo[b]thiophene-2- carboxamide |
| 29 CDD- 3015 | 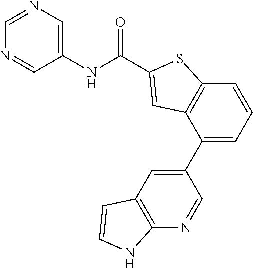 [SMILES: O=C(Nc1cncnc1)c1cc2c(-c3cnc4[nH]ccc4c3)cccc2s1] | N-(pyrimidin-5-yl)-4-(1H-pyrrolo[2,3- b]pyridin-5-yl)benzo[b]thiophene-2- carboxamide |
| 30 CDD- 3016 | 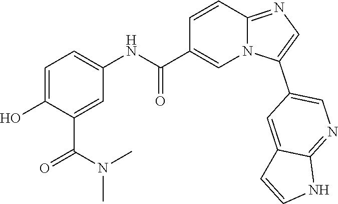 [SMILES: CN(C)C(=O)c1cc(NC(=O)c2ccc3ncc(-c4cnc5[nH]ccc5c4)n3c2)ccc1O] | N-(3-(dimethylcarbamoyl)-4- hydroxyphenyl)-4-(1H-pyrrolo[2,3- b]pyridin-5-yl)benzo[d]thiazole-2- carboxamide |
| 31 CDD- 3017 | 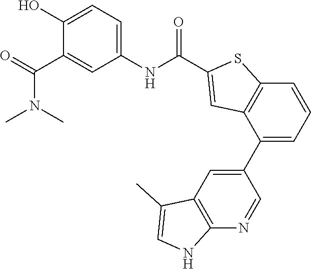 [SMILES: Cc1c[nH]c2ncc(-c3cccc4sc(C(=O)Nc5ccc(O)c(C(=O)N(C)C)c5)cc34)cc12] | N-(3-(dimethylcarbamoyl)-4- hydroxyphenyl)-4-(3-methyl-1H- pyrrolo[2,3-b]pyridin-5- yl)benzo[b]thiophene-2-carboxamide |
| 32 CDD- 3018 | 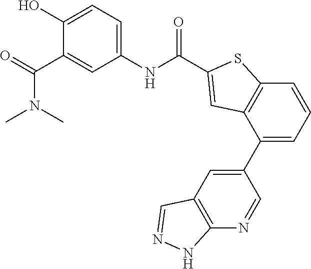 [SMILES: CN(C)C(=O)c1cc(NC(=O)c2cc3c(-c4cnc5[nH]ncc5c4)cccc3s2)ccc1O] | N-(3-(dimethylcarbamoyl)-4- hydroxyphenyl)-4-(1H-pyrazolo[3,4- b]pyridin-5-yl)benzo[b]thiophene-2- carboxamide |
| 33 CDD- 3085 | 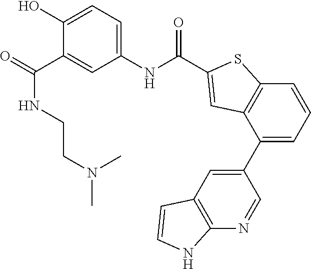 [SMILES: CN(C)CCNC(=O)c1cc(NC(=O)c2cc3c(-c4cnc5[nH]ccc5c4)cccc3s2)ccc1O] | N-(2-(dimethylamino)ethyl)-4-(1H- pyrrolo[2,3-b]pyridin-5- yl)benzo[b]thiophene-2-carboxamide |
| 33 CDD- 3086 | 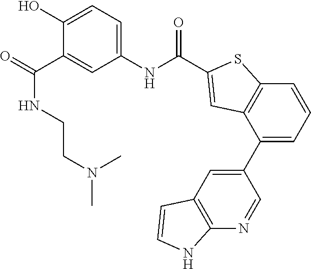 [SMILES: CN(C)CCNC(=O)c1cc(NC(=O)c2cc3c(-c4cnc5[nH]ccc5c4)cccc3s2)ccc1O] | N-(3-((2-(dimethylamino)ethyl)carbamoyl)- 4-hydroxyphenyl)-4-(1H-pyrrolo[2,3- b]pyridin-5-yl)benzo[b]thiophene-2- carboxamide |
| 34 CDD- 3087 | 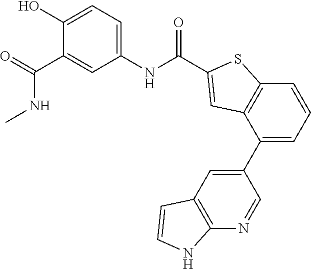 [SMILES: CNC(=O)c1cc(NC(=O)c2cc3c(-c4cnc5[nH]ccc5c4)cccc3s2)ccc1O] | N-(4-hydroxy-3- (methylcarbamoyl)phenyl)-4-(1H- pyrrolo[2,3-b]pyridin-5- yl)benzo[b]thiophene-2-carboxamide |
| 35 CDD- 3111 | 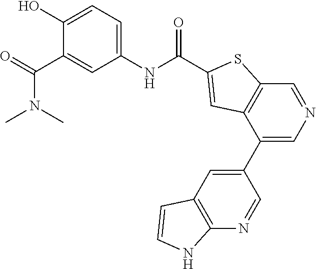 [SMILES: CN(C)C(=O)c1cc(NC(=O)c2cc3c(-c4cnc5[nH]ccc5c4)cncc3s2)ccc1O] | N-(3-(dimethylcarbamoyl)-4- hydroxyphenyl)-4-(1H-pyrrolo[2,3- b]pyridin-5-yl)thieno[2,3-c]pyridine-2- carboxamide |
| 36 CDD- 3112 | 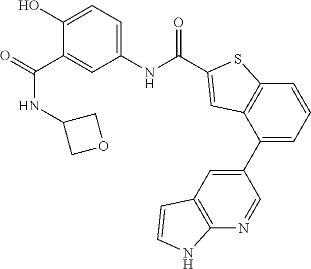 [SMILES: O=C(Nc1ccc(O)c(C(=O)NC2COC2)c1)c1cc2c(-c3cnc4[nH]ccc4c3)cccc2s1] | N-(4-hydroxy-3-(oxetan-3- ylcarbamoyl)phenyl)-4-(1H-pyrrolo[2,3- b]pyridin-5-yl)benzo[b]thiophene-2- carboxamide |
| 37 CDD- 3113 | 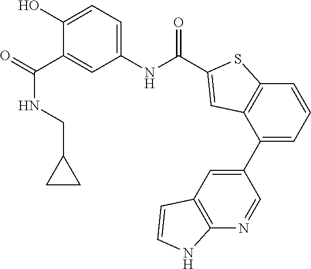 [SMILES: O=C(Nc1ccc(O)c(C(=O)NCC2CC2)c1)c1cc2c(-c3cnc4[nH]ccc4c3)cccc2s1] | N-(3-((cyclopropylmethyl)carbamoyl)-4- hydroxyphenyl)-4-(1H-pyrrolo[2,3- b]pyridin-5-yl)benzo[b]thiophene-2- carboxamide |
| 38 CDD- 3115 | 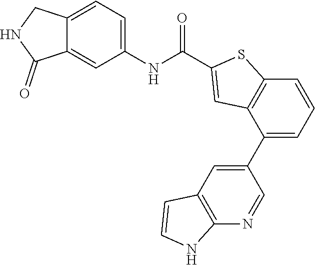 [SMILES: O=C(Nc1ccc2c(c1)C(=O)NC2)c1cc2c(-c3cnc4[nH]ccc4c3)cccc2s1] | N-(3-oxoisoindolin-5-yl)-4-(1H- pyrrolo[2,3-b]pyridin-5- yl)benzo[b]thiophene-2-carboxamide |
| 39 CDD- 3116 | 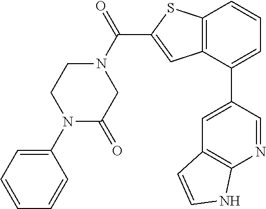 [SMILES: O=C(c1cc2c(-c3cnc4[nH]ccc4c3)cccc2s1)N1CCN(c2ccccc2)C(=O)C1] | 4-(4-(1H-pyrrolo[2,3-b]pyridin-5- yl)benzo[b]thiophene-2-carbonyl)-1- phenylpiperazin-2-one |
| 40 CDD- 3117 | 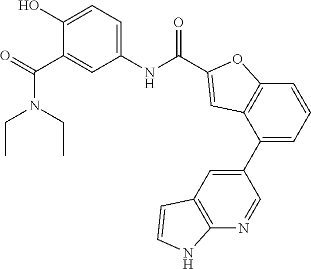 [SMILES: CCN(CC)C(=O)c1cc(NC(=O)c2cc3c(-c4cnc5[nH]ccc5c4)cccc3o2)ccc1O] | N-(3-((diethylamino)methyl)-4- hydroxyphenyl)-4-(1H-pyrrolo[2,3- b]pyridin-5-yl)benzo[b]thiophene-2- carboxamide |
| 41 CDD- 3126 | 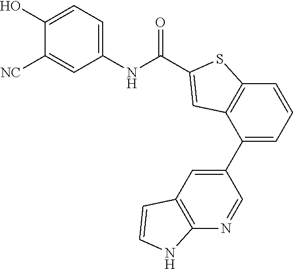 [SMILES: N#Cc1cc(NC(=O)c2cc3c(-c4cnc5[nH]ccc5c4)cccc3s2)ccc1O] | N-(3-cyano-4-hydroxyphenyl)-4-(1H- pyrrolo[2,3-b]pyridin-5- yl)benzo[b]thiophene-2-carboxamide |
| 42 CDD- 2485 | 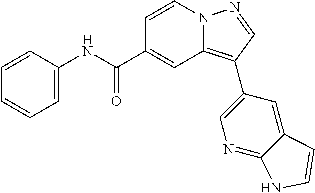 [SMILES: O=C(Nc1ccccc1)c1ccn2ncc(-c3cnc4[nH]ccc4c3)c2c1] | N-phenyl-3-(1H-pyrrolo[2,3-b]pyridin-5- yl)pyrazolo[1,5-a]pyridine-5-carboxamide |
| 43 CDD- 2288 | 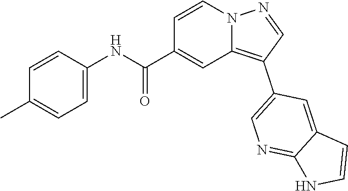 [SMILES: Cc1ccc(NC(=O)c2ccn3ncc(-c4cnc5[nH]ccc5c4)c3c2)cc1] | 3-(1H-pyrrolo[2,3-b]pyridin-5-yl)-N-(p- tolyl)pyrazolo[1,5-a]pyridine-5- carboxamide |
| 44 CDD- 2343 | 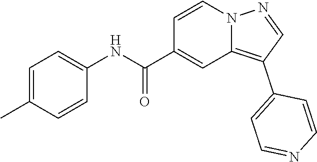 [SMILES: Cc1ccc(NC(=O)c2ccn3ncc(-c4ccncc4)c3c2)cc1] | 3-(pyridin-4-yl)-N-(p-tolyl)pyrazolo[1,5- a]pyridine-5-carboxamide |
| 45 CDD- 2344 | 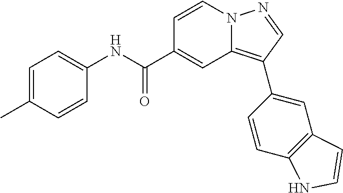 [SMILES: Cc1ccc(NC(=O)c2ccn3ncc(-c4ccc5[nH]ccc5c4)c3c2)cc1] | 3-(1H-indol-5-yl)-N-(p-tolyl)pyrazolo[1,5- a]pyridine-5-carboxamide |
| 46 CDD- 2426 | 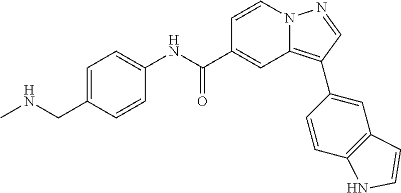 [SMILES: CNCc1ccc(NC(=O)c2ccn3ncc(-c4ccc5[nH]ccc5c4)c3c2)cc1] | 3-(1H-indazol-5-yl)-N-(4- ((methylamino)methyl)phenyl)pyrazolo [1,5-a]pyridine-5-carboxamide |
| 47 CDD- 2388 | 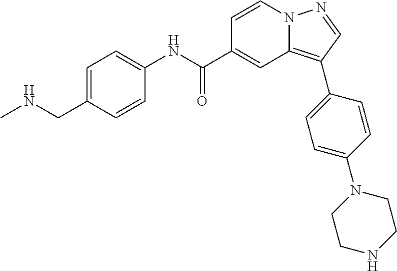 [SMILES: CNCc1ccc(NC(=O)c2ccn3ncc(-c4ccc(N5CCNCC5)cc4)c3c2)cc1] | N-(4-((methylamino)methyl)phenyl)-3-(4- (piperazin-1-yl)phenyl)pyrazolo[1,5- a]pyridine-5-carboxamide |
| 48 CDD- 2489 | 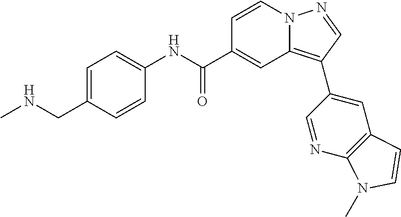 [SMILES: CNCc1ccc(NC(=O)c2ccn3ncc(-c4cnc5c(ccn5C)c4)c3c2)cc1] | 3-(1-methyl-1H-pyrrolo[2,3-b]pyridin-5- yl)-N-(4- ((methylamino)methyl)phenyl)pyrazolo [1,5-a]pyridine-5-carboxamide |
| 49 CDD- 2483 | 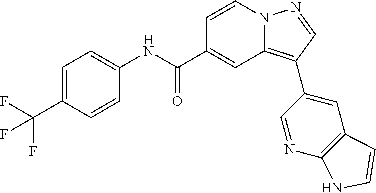 [SMILES: O=C(Nc1ccc(C(F)(F)F)cc1)c1ccn2ncc(-c3cnc4[nH]ccc4c3)c2c1] | 3-(1H-pyrrolo[2,3-b]pyridin-5-yl)-N-(4- (trifluoromethyl)phenyl)pyrazolo[1,5- a]pyridine-5-carboxamide |
| 50 CDD- 2484 | 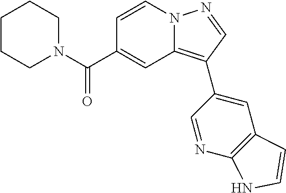 [SMILES: O=C(c1ccn2ncc(-c3cnc4[nH]ccc4c3)c2c1)N1CCCCC1] | (3-(1H-pyrrolo[2,3-b]pyridin-5- yl)pyrazolo[1,5-a\|pyridin-5-yl)(piperidin-1- yl)methanone |
| 51 CDD- 2487 | 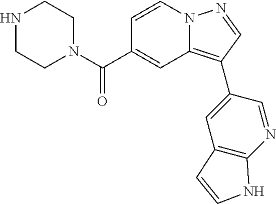 [SMILES: O=C(c1ccn2ncc(-c3cnc4[nH]ccc4c3)c2c1)N1CCNCC1] | (3-(1H-pyrrolo[2,3-b]pyridin-5- yl)pyrazolo[1,5-a]pyridin-5-yl)(piperazin-1- yl)methanone |
| 52 CDD- 2519 | 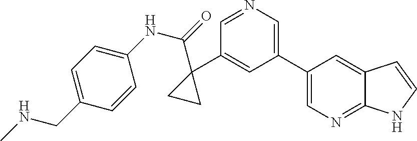 [SMILES: CNCc1ccc(NC(=O)C2(c3cncc(-c4cnc5[nH]ccc5c4)c3)CC2)cc1] | 1-(5-(1H-pyrrolo[2,3-b]pyridin-5- yl)pyridin-3-yl)-N-(4 ((methylamino)methyl)phenyl) cyclopropane-1-carboxamide |
| 53 CDD- 2520 | 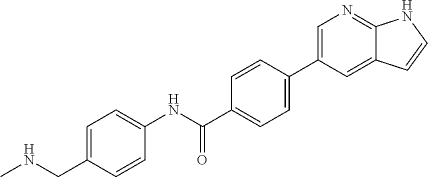 [SMILES: CNCc1ccc(NC(=O)c2ccc(-c3cnc4[nH]ccc4c3)cc2)cc1] | N-(4-((methylamino)methyl)phenyl)-4-(1H- pyrrolo[2,3-b]pyridin-5-yl)benzamide |
| 54 CDD- 2627 | 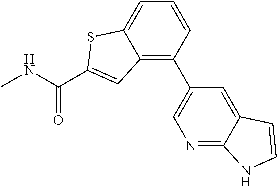 [SMILES: CNC(=O)c1cc2c(-c3cnc4[nH]ccc4c3)cccc2s1] | N-methyl-4-(1H-pyrrolo[2,3-b]pyridin-5- yl)benzo[b]thiophene-2-carboxamide |
| 55 CDD- 2631 | 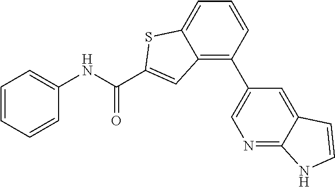 [SMILES: O=C(Nc1ccccc1)c1cc2c(-c3cnc4[nH]ccc4c3)cccc2s1] | N-phenyl-4-(1H-pyrrolo[2,3-b]pyridin-5- yl)benzo[b]thiophene-2-carboxamide |
| 56 CDD- 2629 | 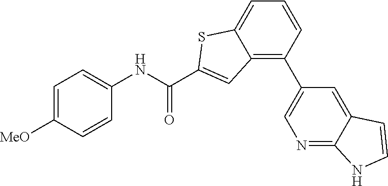 [SMILES: COc1ccc(NC(=O)c2cc3c(-c4cnc5[nH]ccc5c4)cccc3s2)cc1] | N-(4-methoxyphenyl)-4-(1H-pyrrolo[2,3- b]pyridin-5-yl)benzo[b]thiophene-2- carboxamide |
| 57 CDD- 2490 | 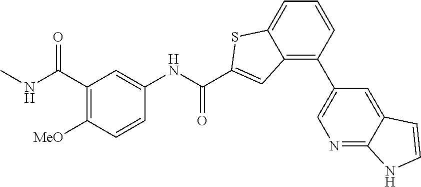 [SMILES: CNC(=O)c1cc(NC(=O)c2cc3c(-c4cnc5[nH]ccc5c4)cccc3s2)ccc1OC] | N-(4-methoxy-3- (methylcarbamoyl)phenyl)-4-(1H- pyrrolo[2,3-b]pyridin-5- yl)benzo[b]thiophene-2-carboxamide |
| 58 CDD- 2636 | 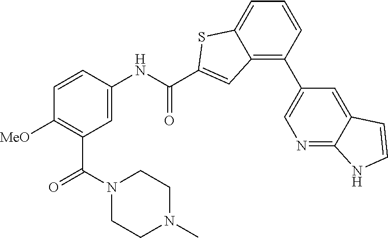 [SMILES: COc1ccc(NC(=O)c2cc3c(-c4cnc5[nH]ccc5c4)cccc3s2)cc1C(=O)N1CCN(C)CC1] | N-(4-methoxy-3-(4-methylpiperazine-1 - carbonyl)phenyl)-4-(1H-pyrrolo[2,3- b]pyridin-5-yl)benzo[b]thiophene-2- carboxamide |
| 59 CDD- 2632 | 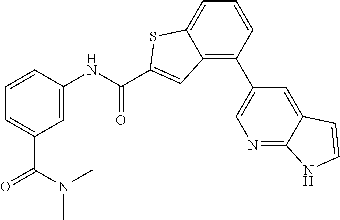 [SMILES: CN(C)C(=O)c1cccc(NC(=O)c2cc3c(-c4cnc5[nH]ccc5c4)cccc3s2)c1] | N-(3-(dimethylcarbamoyl)phenyl)-4-(1H- pyrrolo[2,3-b]pyridin-5- yl)benzo[b]thiophene-2-carboxamide |
| 60 CDD- 2732 | 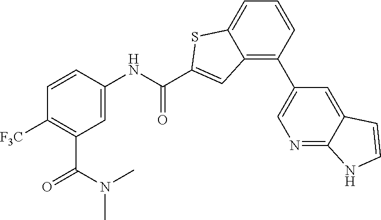 [SMILES: CN(C)C(=O)c1cc(NC(=O)c2cc3c(-c4cnc5[nH]ccc5c4)cccc3s2)ccc1C(F)(F)F] | N-(3-(dimethylcarbamoyl)-4- (trifluoromethyl)phenyl)-4-(1H- pyrrolo[2,3-b]pyridin-5- yl)benzo[b]thiophene-2-carboxamide |
| 61 CDD- 2730 | 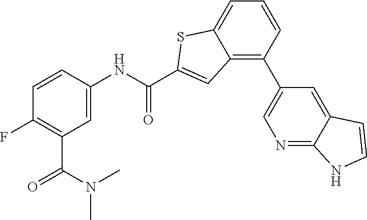 [SMILES: CN(C)C(=O)c1cc(NC(=O)c2cc3c(-c4cnc5[nH]ccc5c4)cccc3s2)ccc1F] | N-(3-(dimethylcarbamoyl)-4-fluorophenyl)- 4-(1H-pyrrolo[2,3-b]pyridin-5- yl)benzo[b]thiophene-2-carboxamide |
| 62 CDD- 1723 | 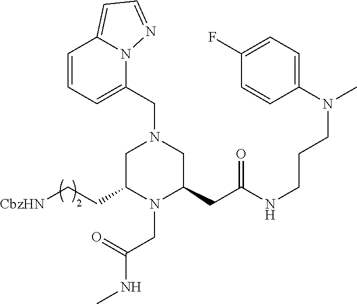 | benzyl (2-((2R,6R)-6-(2-((3-((4- fluorophenyl)(methyl)amino)propyl) amino)-2-oxoethyl)-1-(2-(methylamino)- 2-oxoethyl)-4-(pyrazolo[1,5-a]pyridin-7- ylmethyl)piperazin-2-yl)ethyl)carbamate |
| 63 CDD- 1722 | 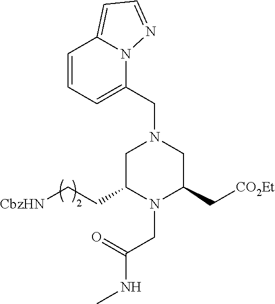 | ethyl 2-((2R,6R)-6-(2- (((benzyloxy)carbonyl)amino)ethyl)-1-(2- (methylamino)-2-oxoethyl)-4- (pyrazolo[1,5-a]pyridin-7- ylmethyl)piperazin-2-yl)acetate |
| 64 CDD- 2009 | 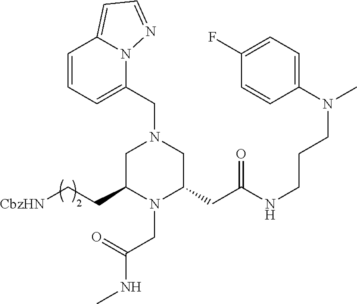 | benzyl (2-((2S,6S)-6-(2-((3-((4- fluorophenyl)(methyl)amino)propyl) amino)-2-oxoethyl)-1-(2-(methylamino)- 2-oxoethyl)-4-(pyrazolo[1,5-a]pyridin-7- ylmethyl)piperazin-2-yl)ethyl)carbamate |
| 65 CDD- 2010 | 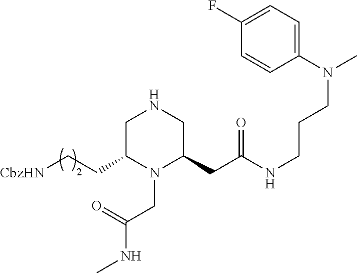 | benzyl (2-((2R,6R)-6-(2-((3-((4- fluorophenyl)(methyl)amino)propyl) amino)-2-oxoethyl)-1-(2-(methylamino)- 2-oxoethyl)piperazin-2-yl)ethyl)carbamate |
| 66 CDD- 2272 | 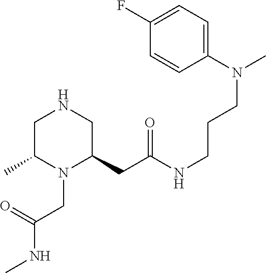 | N-(3-((4- fluorophenyl)(methyl)amino)propyl)-2- ((2R,6R)-6-methyl-1-(2-(methylamino)-2- oxoethyl)piperazin-2-yl)acetamide |

In certain embodiments, each occurrence of optionally substituted alkyl, optionally substituted heteroalkyl, optionally substituted cycloalkyl, optionally substituted heterocycloalkyl, optionally substituted heterocycloalkylenyl, optionally substituted C₁-C₃ alkylenyl, optionally substituted C₃-C₈ cycloalkylenyl, optionally substituted benzyl, optionally substituted aralkyl, optionally substituted aryl, optionally substituted heterocyclyl, and optionally substituted heteroaryl, is independently optionally substituted with at least one substituent selected from the group consisting of C₁-C₆ alkyl, C₃-C₈ cycloalkyl, C₂-C₁₂ heterocycloalkyl, C₁-C₆ hydroxyalkyl, halogen, CN, NO₂ OR^{a}, N(R^{a})(R^{b}), C₁-C₆ haloalkoxy, C₃-C₈ halocycloalkoxy, aryl, heteroaryl, (C₁-C₆ alkylenyl)C(═O)N(R^{a})(R^{b}), (C₁-C₆ alkylenyl)C(═O)OR^{a}, O(C₁-C₃ alkylenyl)C(═O)OR^{a}, O(C₁-C₃ alkylenyl)C(═O)N(R^{a})(R^{b}), C(═O)R^{a}, C(═O)OR^{a}, OC(═O)R^{a}, OC(═O)OR^{a}, SR^{a}—, S(═O)R^{a}, S(═O)₂R^{a}, S(═O)₂N(R^{a})(R^{b}), S(═O)₂NR^{a}C(═O)NHR^{b}, N(R^{a})S(═O)₂R^{b}, N(R^{a})C(═O)R^{b}, and C(═O)NR^{a}R^{b}, wherein R^{a} and R^{b} are each independently selected from the group consisting of H, —C(═O)(C₁-C₆ alkyl), C₁—C₆ alkyl, C₁-C₆ haloalkyl, C₁-C₆ heteroalkyl, C₃-C₈ cycloalkyl, C₂-C₁₂ heterocycloalkyl, C₇-C₁₂ aralkyl, aryl, and heteroaryl.

In another aspect, the present disclosure provides a pharmaceutical composition comprising at least one compound of the present disclosure and a pharmaceutically acceptable carrier.

In certain embodiments, the pharmaceutical composition further comprises at least one additional therapeutically effective agent.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds. Volumes 1-5 and Supplementals (Elsevier Science Publishers, 1989): Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from allyl, benzyl (Bn), benzyloxycarbonyl (Cbz), allyloxycarbonyl (Alloc), methyl, ethyl, t-butyl, t-butyldimethylsilyl (TBDMS), 2-(trimethylsilyl)ethoxycarbonyl (Teoc), t-butyloxycarbonyl (Boc), para-methoxybenzyl (PMB), triphenylmethyl (trityl), acetyl, and fluorenylmethoxycarbonyl (FMOC).

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Methods

In another aspect, the present disclosure provides a method of treating, preventing, and/or ameliorating an inflammatory disease in a subject, the method comprising administering to the subject a therapeutically effective amount of at least one compound of the present disclosure and/or at least one pharmaceutical composition of the present disclosure.

In certain embodiments, JNK11 is selectively inhibited over JNK2 and/or JNK3.

In certain embodiments, JNK11 is inhibited at a similar rate of inhibition as JNK2. In certain embodiments, JNK1 is inhibited at a greater rate of inhibition than JNK2.

In certain embodiments, JNK1 is inhibited at a similar rate of inhibition as JNK3. In certain embodiments, JNK1 is inhibited at a greater rate of inhibition than JNK3.

In certain embodiments, the inflammatory disease is a non-central nervous system (CNS) inflammatory disease.

In certain embodiments, the inflammatory disease is at least one selected from the group consisting of endometriosis, arthritis, pulmonary fibrosis, cancer, type 1 diabetes, and type 2 diabetes. In certain embodiments, the inflammatory disease is endometriosis.

In certain embodiments, progesterone insensitivity in the subject is reduced and/or eliminated. In certain embodiments, pain associated with endometriosis is reduced and/or eliminated. In certain embodiments, normal reproductive function is maintained in the subject.

In certain embodiments, the inflammatory disease is a non-central nervous system (CNS) inflammatory disease.

In certain embodiments, the inflammatory disease is selected from the group consisting of Alzheimer's disease, Parkinson's disease, and amyotrophic lateral sclerosis.

In certain embodiments, the subject is further administered progesterone.

In certain embodiments, the subject is a mammal.

In certain embodiments, the mammal is a human.

In certain embodiments, the human is a female.

In certain embodiments, the compound selectively inhibits JNK1 over JNK2. In certain embodiments, the compound selectively inhibits JNK1 over JNK3. In certain embodiments, the compound selectively inhibits JNK2 over JNK1. In certain embodiments, the compound selectively inhibits JNK2 over JNK3. In certain embodiments, the compound selectively inhibits JNK3 over JNK1. In certain embodiments, the compound selectively inhibits JNK3 over JNK2.

In certain embodiments, the compounds of the present disclosure are suitable to reverse at least one immune disruption that supports the underlying pathophysiology of diseases that increase in the presence of endometriosis.

In certain embodiments, the compounds of the present disclosure are suitable to treat polycystic ovarian disease which is driven by inflammation that causes endocrine disruption and/or insulin resistance.

In certain embodiments, the compounds of the present disclosure are suitable to treat hearing loss, by overcoming glucocorticoid resistance that normally provides protection from cochlear cell death of stress-damaged auditory hair cells.

In certain embodiments, the compounds of the present disclosure are suitable for reducing the size or volume of endometriosis lesions. In certain embodiments, the compounds of the present disclosure are suitable for endometriosis therapy while preserving hypothalamic-pituitary-ovarian steroid control of menstrual cycles and fertility.

In certain embodiments, the compounds of the present disclosure are suitable for reduction of pain associated with endometriosis with preservation of normal reproductive function. In certain embodiments, normal reproductive function includes follicular maturation, ovulation, conception, embryo development, implantation of a blastocyst into uterine decidual tissue, and/or fetal development.

In certain embodiments, the compounds of the present disclosure are suitable to decrease primordial follicle activation, leading to an extension of ovarian lifespan, and delay of the onset of ovarian senescence.

In certain embodiments, the compounds of the present disclosure are suitable to reduce and/or prevent insulin-resistance in subjects with ovarian disease.

In certain embodiments, the method further comprises detecting the inflammatory disease or disorder in a subject. In certain embodiments, the detecting comprises administering to the subject a suitable diagnostic method.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated: each unit containing a predetermined quantity of therapeutic compound calculated to produce the desired therapeutic effect in association with the required pharmaceutical vehicle.

The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9.500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose): fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point. Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously: alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Example 1: Identification of JNK Inhibitors Using DEC-Tec Library Screening

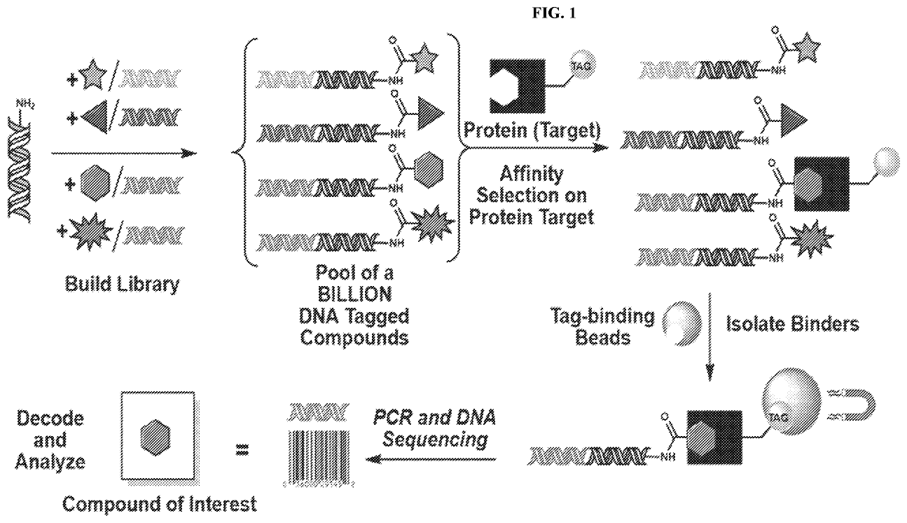

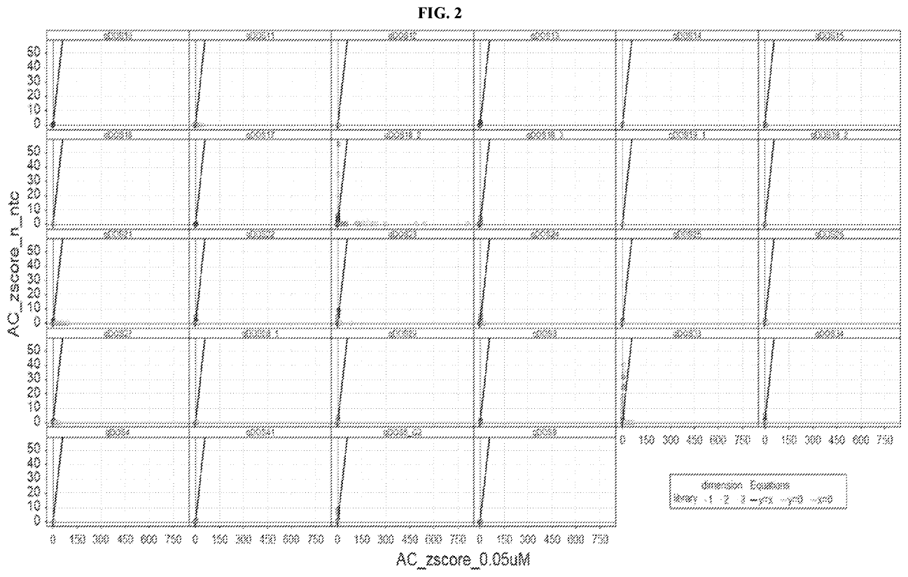

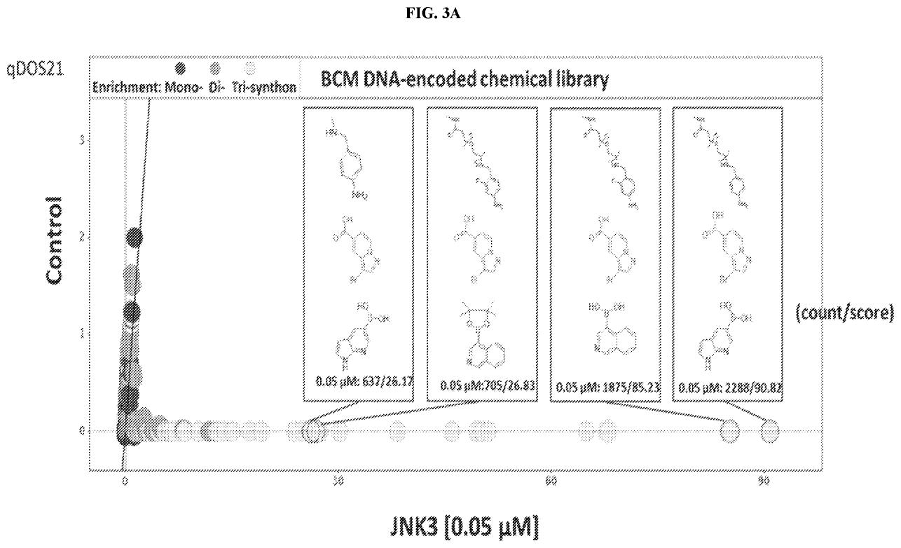

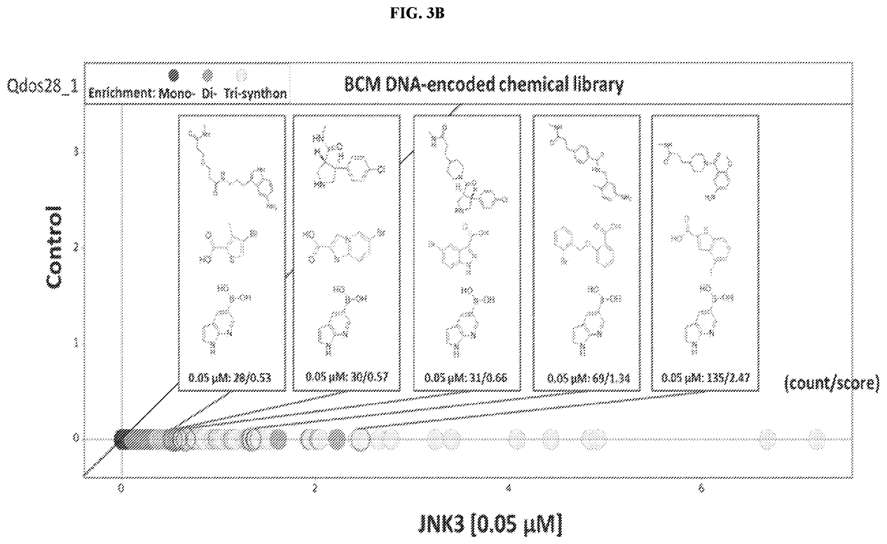

Selections of JNK inhibitors have been conducted and refined from the BCM DEC-Tec chemical library using methods described in FIG. 1 with results in FIG. 2 and FIGS. 3A-3B. Over 4 billion compounds were included in the selection with an objective to identify novel chemical scaffolds and to associate those novel scaffolds as JNK inhibitors. The present disclosure, in one aspect, describes the methods used to identify JNK inhibitors. Seven dominant structures were identified among over 3,000 positives, using established cheminformatic and bioinformatics capabilities. The first observation from DEC-Tec selections performed with 1 M JNK1 enzyme yielded far fewer hits than expected. A depletion step was introduced into our selection methods that dramatically improved selection results.

Subsequently DEC-Tec selections were performed with 0.05 μM JNK3 enzyme with much more manageable results. At this concentration of JNK1, there was large enrichment for compounds in libraries qDOS18_2 to as high as 750-fold enrichment. In qDOS21, the enrichment was approximately 150-fold. According to the target compound profile, desired compounds should have higher affinity for JNK1 than JNK2. Compounds from qDOS24 were enriched with JNK2, while compounds from qDOS21 were not enriched with JNK2. These results suggested an inherent selectivity preference for JNK1 vs JNK2 in qDOS21. By comparison to JNK1, compounds in qDOS18_2 demonstrated high non-specific binding (y-axis) relative to enrichment (x-axis). Finally, DEC-Tec selections performed were performed with JNK3 and results from the two libraries with the greatest enrichment are presented (FIGS. 3A-3B).

Library qDOS21 again showed very good enrichment of compounds selected with JNK3 and qDOS28_1 had less, but good enrichment with JNK3. Based on overlapping scaffolds within qDOS21 that were represented in both JNK1 and JNK3 selections, hits from this library were resynthesized. These results are described in greater detail elsewhere herein. Briefly, the results described herein demonstrate the use of the DEC-Tec platform to establish selectivity among isoforms of JNKs. Selected compounds in libraries that were over-represented in multiple selections, and among libraries with promiscuous ligands, were rejected. This was the case with qDOS18_2 hits.

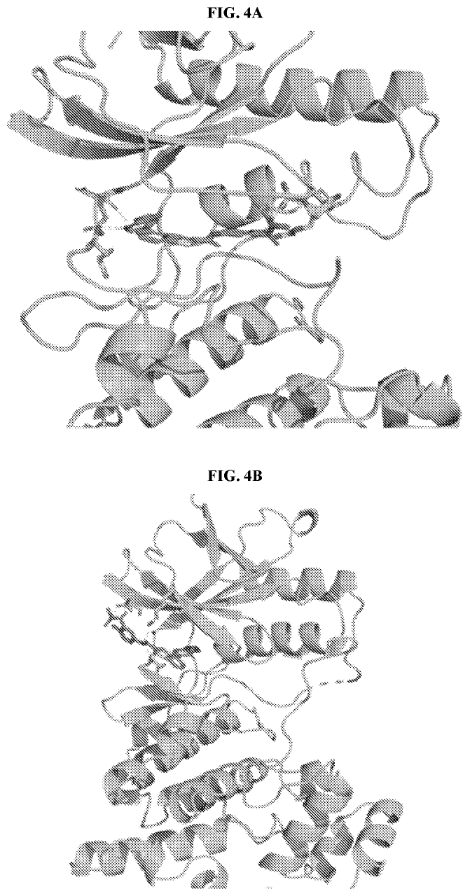

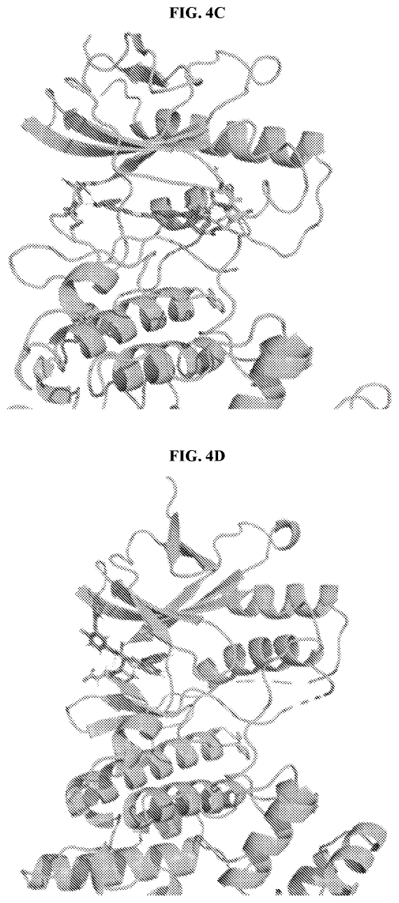

The following crystal structures of human JNK1 and JNK3 (i.e., 4QTD, 4WHZ, 3ELJ, 4AWI, 4L7F, 3PZE, 4HYU, 4E73, 4IZY, 2H96, 2NO3, 2G01, 2GMX, 3TTJ, 3TTI) have been overlaid to observe possible side chain conformations. Their small molecule ligands were optimized, and a pharmacophore model was created from these plus four other known binders described in the literature. The modeling generated a common overlap of several compounds from the DEC-Tec selection described herein with known structures (FIGS. 4A-4D).

### Example 2: Chemical Synthesis of Compounds Identified in qDOS21 and qDOS28_1 Libraries

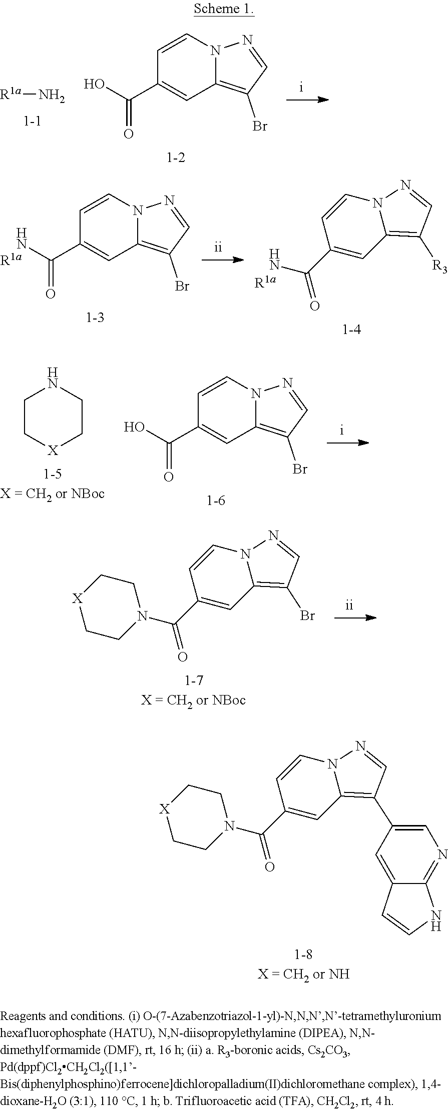

### General Procedure for Amide Synthesis (i.e., Step (i) in Scheme 1)

To a stirred solution of 3-Bromopyrazolo[1,5-a]pyridine-5-carboxylic acid (100 mg, 0.4 mmol, 1.0 eq) in anhydrous DMF (2 mL) was added substituted amines (1.5 eq), HATU (236 mg, 0.6 mmol, 1.2 eq), DIPEA (217 μL, 1.24 mmol, 3.0 eq), and the resulting mixture was stirred at room temperature for 14 h. After completion, the reaction (monitored by TLC and LC-MS) was quenched by adding water and extracted with 20 mL of ethyl acetate (EtOAc) thrice. The combined organic layers were washed with brine solution (NaCl), dried over anhydrous sodium sulfate (Na₂SO₄), filtered, and concentrated under reduced pressure to give a crude reaction mixture. It was purified by silica gel column chromatography (Teledyne ISCO CombiFlash system) using hexane-EtOAc (100-0 to 0-100) to afford the desired compound in moderate to good yields (63-88%).

### General Procedure for Suzuki Cross-Coupling Reaction (i.e., Step (ii) in Scheme 1)

A 10 mL oven dried microwave vial equipped with magnetic stir bar was charged with the 1.0 eq of aryl halide intermediate, boronic acid (1.2 eq), Cs₂CO₃ (2.5 eq), and Pd(dppf)Cl₂·CH₂Cl₂ (0.1 eq) followed by addition of 1,4-dioxane-H₂O (3:1, 4 mL). The composition of a vial was sealed with a microwave cap, purged with nitrogen for 25 mins, and then irradiated the vial at 110° C. under microwave conditions for 1 h. After completing the reaction (monitored by TLC and LC-MS), the crude material containing unwanted catalyst was removed by filtered using celite. The clear solution was extracted with 20 mL of ethyl acetate (EtOAc) thrice, the combined organic layers were washed with brine solution (NaCl), and dried over anhydrous sodium sulfate (Na₂SO₄), filtered, and concentered under reduced pressure to give a crude reaction mixture. It was purified by normal phased silica gel column chromatography (Teledyne ISCO CombiFlash system) CH₂Cl₂-MeOH (100-0 to 0-10) as eluent to afford the Boc-protected compounds. Further, it was reacted with TFA in CH₂Cl₂ to remove the Boc protection and purified by reversed phase column chromatography (Teledyne ISCO CombiFlash system) using solvent CH₃CN—H₂O (0-90 to 95-5) to obtain the desired compounds in 32-72% yield.

The following exemplary compounds were prepared according to the synthesis depicted Scheme 1.

### N-methyl-3-(1H-pyrrolo[2,3-b]pyridin-5-yl)pyrazolo[1,5-a]pyridine-5-carboxamide (CDD-2574)

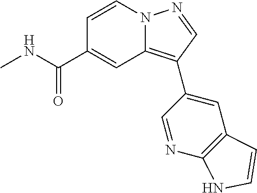 [SMILES: CNC(=O)c1ccn2ncc(-c3cnc4[nH]ccc4c3)c2c1]

Yield 57% (63 mg); Light yellow solid; ¹H NMR (600 MHz, DMSO) δ 11.75 (s, 1H), 8.80 (d, J=7.1 Hz, 2H), 8.61-8.58 (mi 1H), 8.45 (d, J=16.5 Hz, 2H), 8.27 (d, J=2.2 Hz, 1H), 7.54 (t, J=2.8 Hz, 1H), 7.33 (dd, J=7.2, 1.9 Hz, 1H), 6.55-6.52 (m, 1H), 2.84 (d, J=4.5 Hz, 3H); ¹³C NMR (151 MHz, DMSO) δ 165.36, 148.01, 142.20, 141.58, 135.64, 130.36, 129.51, 127.36, 126.69, 120.55, 120.32, 116.65, 113.24, 111.07, 100.43, 26.76; HRMS (ESI) m/z calcd for C₁₆H₁₄N₅O [M+H]⁺ 292.1198, found 292.1187.

### N-phenyl-3-(1H-pyrrolo[2,3-b]pyridin-5-yl)pyrazolo[1,5-a]pyridine-5-carboxamide (CDD-2485)

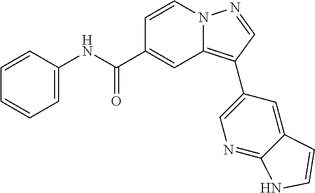 [SMILES: O=C(Nc1ccccc1)c1ccn2ncc(-c3cnc4[nH]ccc4c3)c2c1]

Yield 32% (40 mg); Light yellow solid; ¹H NMR (600 MHz, DMSO) δ 11.77 (s, 1H), 10.52 (s, 1H), 8.87 (d, J=7.3 Hz, 1H), 8.65 (d, J=2.1 Hz, 1H), 8.61 (s, 1H), 8.51 (s, 1H), 8.32 (d, J=2.2 Hz, 1H), 7.78 (d, J=8.0 Hz, 2H), 7.55 (t, J=3.0 Hz, 1H), 7.45-7.42 (m, 1H), 7.39 (t, J=7.8 Hz, 2H), 7.14 (t, J=7.4 Hz, 1H), 6.56-6.53 (m, 1H); ¹³C NMR (151 MHz, DMSO) δ 164.23, 148.06, 142.27, 141.73, 139.29, 135.50, 130.58, 129.62, 129.14, 127.40, 126.83, 124.48, 121.18, 120.53, 120.37, 117.68, 113.68, 111.37, 100.48; HRMS (ESI) m/z calcd for C₂₁H₁₆N₅O [M+H]⁺ 354.1354, found 354.1349.

### 3-(1H-pyrrolo[2, 3-b]pyridin-5-yl)-N-(p-tolyl)pyrazolo[, 5-a]pyridine-5-carboxamide (CDD-2288)

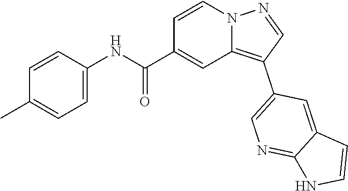 [SMILES: Cc1ccc(NC(=O)c2ccn3ncc(-c4cnc5[nH]ccc5c4)c3c2)cc1]

Yield 60% (27 mg); Light yellow solid; ¹H NMR (600 MHz, DMSO) δ 11.76 (s, 1H), 10.47 (s, 1H), 8.85 (d, J=7.3 Hz, 1H), 8.62 (d, J=30.4 Hz, 2H), 8.50 (s, 1H), 8.33 (s, 1H), 7.66 (d, J=8.0 Hz, 2H), 7.54 (t, J=2.9 Hz, 1H), 7.43 (d, J=7.3 Hz, 1H), 7.18 (d, J=8.0 Hz, 2H), 6.54 (d, J=3.4 Hz, 1H), 2.29 (s, 3H); 13C NMR (151 MHz, DMSO) δ 164.01, 148.04, 142.25, 141.70, 136.75, 135.53, 133.47, 130.65, 129.57, 129.51, 127.38, 126.83, 121.22, 120.54, 120.38, 117.57, 113.63, 111.38, 100.47, 20.98: HRMS (ESI) m/z calcd for C₂₂H₁₈N₅O [M+H]⁺ 368.1511, found 368.1510.

### 3-(isoquinolin-4-yl)-N-(p-tolyl)pyrazolo[1,5-a]pyridine-5-carboxamide (CDD-2325)

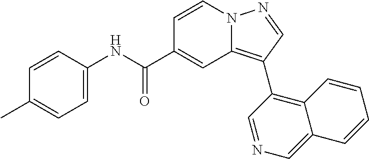 [SMILES: Cc1ccc(NC(=O)c2ccn3ncc(-c4cncc5ccccc45)c3c2)cc1]

Yield 63% (70 mg); Light yellow solid; ¹H NMR (600 MHz, DMSO) δ 10.32 (s, 1H), 9.41-9.38 (m. 1H), 8.96 (dd, J=7.3, 0.9 Hz. 1H), 8.69 (s, 1H), 8.49 (s, 1H), 8.29-8.23 (m, 2H), 7.96 (dd, J=8.3, 1.2 Hz, 1H), 7.83 (ddd, J=8.3, 6.8, 1.4 Hz, 1H), 7.77 (ddd, J=8.0, 6.8, 1.2 Hz, 1H), 7.60-7.56 (m, 2H), 7.51 (dd, J=7.3, 1.9 Hz, 1H), 7.14 (d, J=8.2 Hz, 2H), 2.27 (s, 3H), 1.99 (s, 1H); ¹³C NMR (151 MHz, DMSO) δ 170.79, 163.70, 152.30, 143.85, 143.75, 137.33, 136.59, 134.28, 133.51, 131.72, 131.12, 129.70, 129.48, 128.76, 128.67, 128.20, 124.58, 123.51, 121.14, 117.14, 111.78, 109.48, 40.44, 40.30, 40.16, 40.02, 39.88, 39.74, 39.60, 20.94; HRMS (ESI) m/z calcd for C₂₄H₁₉N₄O [M+H]379.1558, found 379.1544.

### N-(4-((methylamino)methyl)phenyl)-3-(1H-pyrrolo[2,3-b]pyridin-5-yl)pyrazolo[1,5-a]pyridine-5-carboxamide (CDD-2346)

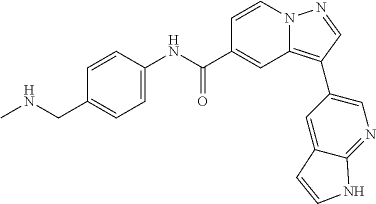 [SMILES: CNCc1ccc(NC(=O)c2ccn3ncc(-c4cnc5[nH]ccc5c4)c3c2)cc1]

Yield 53% (46 mg); Light yellow solid; ¹H NMR (600 MHz, DMSO) δ 11.77 (s, 1H), 10.64 (s, 1H), 8.88 (d, J=7.3 Hz, 1H), 8.81 (s, 1H), 8.62 (dd, J=16.6, 2.0 Hz, 2H), 8.52 (s, 1H), 8.32 (d, J=2.1 Hz, 1H), 7.86-7.82 (m, 2H), 7.55 (1, J=2.9 Hz, 1H). 7.48 (d, J=8.5 Hz. 2H), 7.42 (dd, J=7.3, 1.9 Hz. 1H), 6.54 (dd, J=3.4, 1.8 Hz. 1H), 4.12 (s, 2H), 2.58 (s, 3H); ¹³C NMR (151 MHz, DMSO) δ 164.34, 148.06, 142.26, 141.78, 139.93, 135.48, 130.84, 130.34, 129.68, 127.82, 127.43, 126.85, 121.14, 120.48, 120.37, 117.80, 113.79, 111.29, 100.47, 51.46, 32.48; HRMS (ESI) m/z calcd for C₂₃H₂₁N₆O [M+H]⁺ 397.1776, found 397.1771.

### 3-(isoquinolin-4-yl)-N-(4-((methylamino)methyl)phenyl)pyrazolo[1,5-a]pyridine-5-carboxamide (CDD-2347)

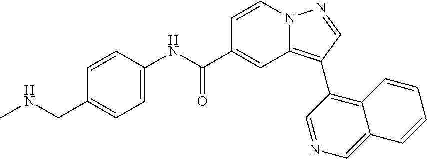 [SMILES: CNCc1ccc(NC(=O)c2ccn3ncc(-c4cncc5ccccc45)c3c2)cc1]

Yield 69% (80 mg); White solid ¹H NMR (600 MHz, MeOD) δ 8.80 (dd, J=7.4, 2.4 Hz. 1H), 8.39 (d. J=2.1 Hz. 1H), 8.28 (d. J=8.2 Hz. 1H), 8.21 (s, 1H), 8.02 (d, J=8.4 Hz, 1H), 7.88 (t, J=7.7 Hz, 1H), 7.80 (dd, J=22.5, 8.0 Hz, 3H). 7.53 (d. J=7.3 Hz, 1H). 7.46 (d, J=8.1 Hz, 2H), 4.17 (s, 2H), 2.73 (s, 3H); ¹³C NMR (151 MHz, MeOD) δ 164.75, 143.03, 139.62, 137.55, 132.00, 130.99, 130.11, 128.85, 128.53, 128.22, 127.03, 124.19, 121.12, 116.82, 111.07, 51.76, 31.58; HRMS (ESI) m/z calcd for C₂₅H₂₂N₅O [M+H]⁺ 408.1824, found 408.1821.

### 3-(pyridin-4-yl)-N-(p-tolyl)pyrazolo[1,5-a]pyridine-5-carboxamide (CDD-2343)

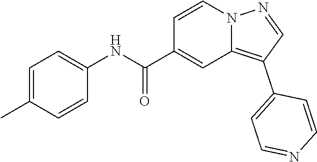 [SMILES: Cc1ccc(NC(=O)c2ccn3ncc(-c4ccncc4)c3c2)cc1]

Yield 62% (46 mg); White solid; ¹H NMR (600 MHz, DMSO) δ 10.49 (s, 1H), 8.92 (d, J=7.2 Hz, 1H), 8.69 (d, J=34.2 Hz, 4H), 7.86 (s. 2H), 7.67 (d. J=8.0 Hz. 2H), 7.51-7.45 (m, 1H), 7.20 (d, J=8.0 Hz, 2H), 2.30 (s, 3H): ¹³C NMR (151 MHz, DMSO) δ 163.82, 150.71, 142.69, 140.07, 136.65, 136.09, 133.63, 132.30, 130.16, 129.56, 121.37, 121.21, 117.42, 112.18, 111.86, 111.84, 20.99; HRMS (ESI) in z calcd for C₂₀H₁₇N₄O [M+H]⁺ 329.1402, found 329.1388.

### 3-(1H-indol-5-yl)-N-(p-tolyl)pyrazolo[1,5-a]pyridine-5-carboxamide (CDD-2344)

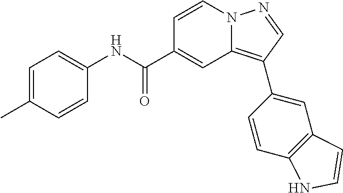 [SMILES: Cc1ccc(NC(=O)c2ccn3ncc(-c4ccc5[nH]ccc5c4)c3c2)cc1]

Yield 63% (29 mg); Light yellow solid; ¹H NMR (600 MHz, DMSO) δ 11.17 (s, 1H), 10.42 (s, 1H), 8.82 (d, J=7.3 Hz, 1H), 8.56 (d, J=2.0 Hz, 1H), 8.40 (s, 1H), 7.91 (d, J=1.7 Hz, 1H), 7.66 (d, J=8.1 Hz, 2H), 7.54 (d, J=8.3 Hz, 1H), 7.47 (dd, J=8.4, 1.7 Hz, 1H), 7.42-7.35 (m, 2H), 7.18 (d, J=8.2 Hz, 2H), 6.52 (t, J=2.5 Hz, 1H), 2.30 (s, 3H); ¹³C NMR (151 MHz, DMSO) δ 164.14, 141.49, 136.82, 135.51, 135.25, 133.41, 130.24, 129.52, 129.45, 128.84, 126.52, 123.29, 121.49, 121.13, 121.03, 118.97, 117.96, 116.83, 112.55, 110.99, 101.79, 20.98; HRMS (ESI) m/z calcd for C₂₃H₁₉N₄O [M+H]J 367.1558, found 367.1547.

### 3-(1H-indazol-5-yl)-N-(4-((methylamino)methyl)phenyl)pyrazolo[1,5-a]pyridine-5-carboxamide (CDD-2426)

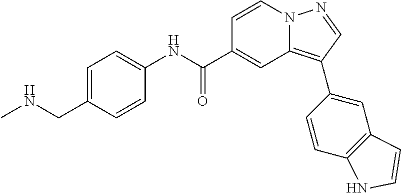 [SMILES: CNCc1ccc(NC(=O)c2ccn3ncc(-c4ccc5[nH]ccc5c4)c3c2)cc1]

Yield 36% (24 mg); Light yellow solid; ¹H NMR (600 MHz, DMSO) δ 10.86 (s, 1H), 9.51-9.37 (m. 2H), 8.84 (d, J=7.2 Hz, 1H), 8.65 (d, J=1.9 Hz. 1H), 8.47 (s, 1H), 8.18 (d, J=27.2 Hz, 2H), 7.89 (d, J=8.1 Hz, 2H), 7.84-7.76 (m, 1H), 7.68 (d, J=8.6 Hz, 1H), 7.55 (d, J=8.2 Hz, 2H), 7.45 (dd, J=7.2, 1.9 Hz. 1H), 4.07 (t, J=5.9 Hz, 2H), 2.51 (d, J=5.2 Hz, 3H); ¹³C NMR (151 MHz, DMSO) δ 164.48, 141.74, 139.96, 139.51, 135.33, 134.03, 130.92, 130.39, 129.59, 127.72, 126.86, 124.78, 124.06, 121.05, 118.95, 118.10, 115.81, 111.47, 111.34, 51.19, 32.17; HRMS (ESI) m/z calcd for C₂₃H₂₁N₆O [M+H]⁺ 397.1776, found 397.1777.

### 3-(2-aminopyrimidin-5-yl)-N-(4-((methylamino)methyl)phenyl)pyrazolo[1,5-a]pyridine-5-carboxamide (CDD-2387)

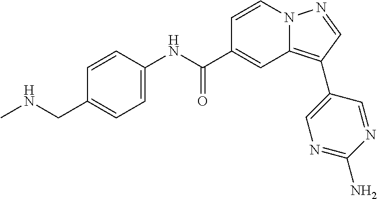 [SMILES: CNCc1ccc(NC(=O)c2ccn3ncc(-c4cnc(N)nc4)c3c2)cc1]

Yield 48% (22 mg); Light yellow solid; ¹H NMR (600 MHz, DMSO) δ 10.43 (s, 1H), 8.79 (d, J=7.3 Hz, 1H), 8.66 (s. 2H), 8.50 (s, 1H), 8.41 (s, 1H), 7.68 (dd, J=8.2, 4.2 Hz, 2H), 7.42-7.35 (m, 1H), 7.31 (d, J=8.1 Hz, 2H), 6.73 (d, J=3.0 Hz, 1H), 3.61 (s, 2H), 2.25 (s, 3H); ¹³C NMR (151 MHz, DMSO) δ 163.85, 162.77, 156.64, 140.90, 137.55, 136.76, 135.12, 130.40, 129.55, 128.69, 121.07, 120.96, 117.38, 115.44, 111.43, 109.87, 54.98, 35.78; HRMS (ESI) m/z calcd for C₂₀H₂₀N₇O [M+H]⁺ 374.1729, found 374.172.

### N-(4-((methylamino)methyl)phenyl)-3-(4-(piperazin-1-yl)phenyl)pyrazolo[1,5-a]pyridine-5-carboxamide (CDD-2388)

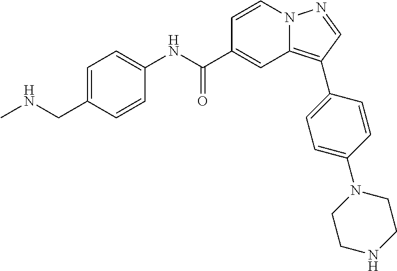 [SMILES: CNCc1ccc(NC(=O)c2ccn3ncc(-c4ccc(N5CCNCC5)cc4)c3c2)cc1]

Yield 46% (30 mg); Light yellow solid; ¹H NMR (600 MHz, DMSO) δ 10.98 (s, 1H), 9.66 (s, 2H), 8.80 (d, J=7.3 Hz, 1H), 8.59 (d, =2.0 Hz, 1H), 8.40 (s, 1H), 7.90 (d, J=8.3 Hz, 2H), 7.74 (d, J=8.3 Hz, 2H), 7.57 (d, J=8.4 Hz, 2H), 7.45 (dd, J=7.3, 1.9 Hz, 1H), 7.12 (d, J=8.5 Hz, 2H), 4.04 (s, 2H), 3.46 (dd, J=6.7, 3.9 Hz, 4H), 3.20-3.11 (m, 4H), 2.46 (s. 3H); ¹³C NMR (151 MHz, DMSO) δ 164.45, 148.73, 141.30, 139.98, 135.04, 130.92, 130.20, 129.53, 128.38, 127.70, 124.54, 120.96, 118.25, 117.30, 115.05, 111.32, 51.10, 49.02, 46.00, 42.67, 32.07: HRMS (ESI) m/z calcd for C₂₆H₂₉N₆O [M+H]⁺ 441.2402, found 441.2397.

### 3-(1-methyl-JH-pyrrolo[2,3-b]pyridin-5-yl)-N-(4-((methylamino)methyl)phenyl)pyrazolo[1,5-a]pyridine-5-carboxamide (CDD-2489)

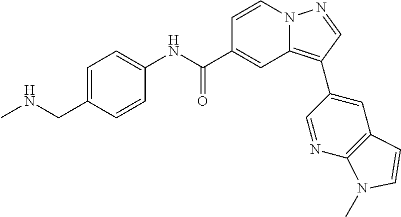 [SMILES: CNCc1ccc(NC(=O)c2ccn3ncc(-c4cnc5c(ccn5C)c4)c3c2)cc1]

Yield 72% (50 mg); Light yellow solid; ¹H NMR (600 MHz, DMSO) δ 10.65 (s, 1H), 8.88 (d, J=7.3 Hz, 3H). 8.70 (d, J=2.1 Hz, 1H). 8.63-8.60 (m. 1H), 8.52 (s. 1H), 8.33 (d. J=2.1 Hz, 1H), 7.84 (d, J=8.4 Hz, 2H), 7.60 (d, J=3.4 Hz, 1H), 7.49 (d, J=8.3 Hz, 2H), 7.43 (dd, J=7.2, 1.9 Hz, 1H), 6.56 (d, J=3.4 Hz, 1H), 4.12 (t, J=5.9 Hz, 2H), 3.89 (s, 3H), 2.58 (t, J=5.4 Hz, 3H); ¹³C NMR (151 MHz, DMSO) δ 163.85, 146.49, 141.54, 141.34, 139.46, 134.99, 130.89, 130.37, 129.89, 129.22, 127.23, 126.73, 120.67, 120.22, 120.03, 117.25, 113.10, 110.84, 99.02, 50.94, 31.97, 30.91; HRMS (ESI) m/z calcd for C₂₄H₂₃N₆O [M+H]⁺ 411.1933, found 411.1923.

### N-(4-((dimethylamino)methyl)phenyl)-3-(1H-pyrrolo[2,3-b]pyridin-5-yl)pyrazolo[1,5-a]pyridine-5-carboxamide (CDD-2482)

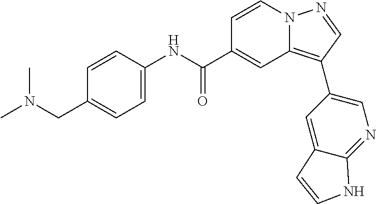 [SMILES: CN(C)Cc1ccc(NC(=O)c2ccn3ncc(-c4cnc5[nH]ccc5c4)c3c2)cc1]

Yield 58% (96 mg); Light yellow solid; ¹H NMR (600 MHz, DMSO) δ 11.76 (s, 1H), 10.54 (d, J=4.1 Hz, 1H), 8.86 (d, J=7.3 Hz, 1H), 8.64 (d, J=1.9 Hz, 1H), 8.60 (s, 1H), 8.50 (s, 1H), 8.32 (d, J=2.1 Hz, 1H), 7.75 (d, J=8.1 Hz, 2H), 7.54 (t, J=2.9 Hz, 1H), 7.42 (dd, J=7.3, 1.8 Hz, 1H), 7.32 (d, J=8.2 Hz, 2H), 6.54 (dd, J=3.4, 1.8 Hz, 1H). 3.51 (s, 2H), 2.23 (s, 6H); ¹³C NMR (151 MHz, DMSO) δ 164.19, 148.05, 142.26, 141.72, 138.46. 135.49, 130.57, 129.91, 129.62, 127.39, 126.83, 120.95, 120.52, 120.37, 117.67, 113.68, 111.36, 100.47, 62.81, 49.07, 44.78: HRMS (ESI) m/z calcd for C₂₄H₂₃N₆O [M+H]⁺ 411.1933, found 411.1927.

### N-(4-(methylcarbamoyl)phenyl)-3-(1H-pyrrolo[2,3-b]pyridin-5-yl)pyrazolo[1,5-a]pyridine-5-carboxamide (CDD-2461)

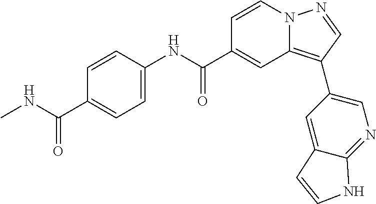 [SMILES: CNC(=O)c1ccc(NC(=O)c2ccn3ncc(-c4cnc5[nH]ccc5c4)c3c2)cc1]

Yield 38% (21 mg); Light yellow solid; ¹H NMR (600 MHz, DMSO) δ 11.76 (s. 1H), 10.69 (s, 1H), 8.87 (d, J=7.3 Hz, 1H), 8.63 (dd, J=9.4, 2.0 Hz, 2H), 8.51 (s, 1H). 8.38 (q, J=4.5 Hz, 1H), 8.32 (d, 1=2.2 Hz, 1H), 7.86 (s, 4H), 7.54 (t, =2.9 Hz, 1H), 7.42 (dd, J=7.3, 1.9 Hz, 1H), 6.53 (dd, J=3.5, 1.8 Hz, 1H), 2.79 (d, J=4.5 Hz, 3H); ¹³C NMR (151 MHz, DMSO) δ 166.57, 164.47, 148.06, 142.27, 141.78, 135.45, 130.30, 130.22, 129.68, 128.25, 127.42, 126.86, 120.48, 120.37, 120.22, 117.91, 113.83, 111.32, 100.48, 26.69; HRMS (ESI) m/z calcd for C₂₃H₁₉N₆O₂ [M+H]⁺ 411.1569. found 411.1569.

### 3-(1H-pyrrolo[2,3-b]pyridin-5-yl)-N-(4-(trifluoromethyl)phenyl)pyrazolo[1,5-a]pyridine-5-carboxamide (CDD-2483)

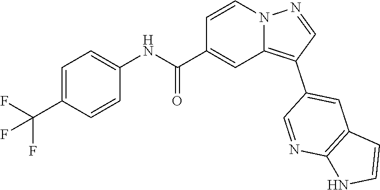 [SMILES: O=C(Nc1ccc(C(F)(F)F)cc1)c1ccn2ncc(-c3cnc4[nH]ccc4c3)c2c1]

Yield 49% (23 mg); Light yellow solid; ¹H NMR (600 MHz, DMSO) δ 11.77 (s, 1H), 10.82 (s, 1H), 8.88 (d, J=7.3 Hz, 1H), 8.64 (s, 2H), 8.52 (s, 1H), 8.33 (s. 1H), 8.03 (d, J=8.5 Hz. 2H), 7.76 (d. J=8.3 Hz. 2H), 7.55 (s, 1H), 7.43 (d, J=7.4 Hz, 1H), 6.54 (s, 1H); ¹³C NMR (151 MHz, DMSO) δ 164.74, 148.08, 143.01, 142.28, 141.80, 135.42, 130.05, 129.72, 127.43, 126.88, 126.47, 126.44, 126.42, 126.39, 120.90, 120.45, 120.37, 118.11, 113.93, 111.28, 100.48; HRMS (ESI) m/z calcd for C₂₂H₁₅F3N₅O [M+H]⁺ 422.1228, found 422.1215.

### N-(4-fluorophenyl)-3-(1H-pyrrolo[2,3-b]pyridin-5-yl)pyrazolo[1,5-a]pyridine-5-carboxamide (CDD-2486)

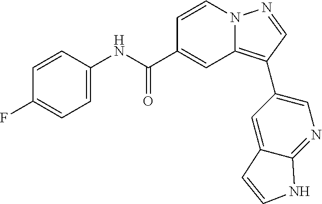 [SMILES: O=C(Nc1ccc(F)cc1)c1ccn2ncc(-c3cnc4[nH]ccc4c3)c2c1]

Yield 69% (87 mg); Light yellow solid; ¹H NMR (600 MHz, DMSO) δ 11.76 (s, 1H), 10.56 (s, 1H), 8.87 (d, J=7.3 Hz, 1H), 8.65-8.62 (m, 1H), 8.60 (s, 1H). 8.51 (s, 1H), 8.33-8.30 (m, 1H), 7.79 (dd, J=8.8, 5.0 Hz, 2H), 7.55 (d, J=3.0 Hz, 1H). 7.42 (d, J=7.6 Hz, 1H), 7.23 (t, J=8.7 Hz, 2H), 6.56-6.52 (m, 1H): ¹³C NMR (151 MHz, DMSO) δ 164.15, 159.76, 158.17, 148.06, 142.26, 141.75, 135.64, 135.62, 135.50, 130.41, 129.65, 127.41, 126.83, 123.09, 123.04, 120.50, 120.37, 117.67, 115.81, 115.66, 113.69, 111.30, 100.47: HRMS (ESI) m/z calcd for C₂₁H₁₅FN₅O [M+H]⁺ 372.126, found 372.1251.

### (3-(1H-pyrrolo[2,3-b]pyridin-5-yl)pyrazolo[1,5-a]pyridin-5-yl)(piperidin-1-yl)methanone (CDD-2484)

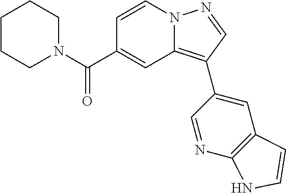 [SMILES: O=C(c1ccn2ncc(-c3cnc4[nH]ccc4c3)c2c1)N1CCCCC1]

Yield 47% (50 mg); White solid; ¹H NMR (600 MHz, DMSO) δ 11.72 (s, 1H), 8.79 (d, J=7.1 Hz, 1H), 8.54 (d, J=2.1 Hz, 1H), 8.46 (s, 1H), 8.24 (d, J=2.1 Hz, 1H), 7.94 (s, 1H), 7.52 (t, J=2.9 Hz, 1H), 6.92 (dd, J=7.1, 1.7 Hz, 1H), 6.51 (dd. J=3.4, 1.8 Hz, 1H). 3.60 (s. 2H), 3.38 (s. 2H), 3.18 (d, J=5.2 Hz. 1H), 1.63-1.47 (m, 6H); ¹³C NMR (151 MHz, DMSO) δ 167.48, 147.92, 142.04, 141.46, 135.67, 132.71, 129.80, 127.27, 126.45, 120.61, 120.34, 115.87, 112.23, 111.47, 100.44, 49.07, 48.55, 25.62, 24.49; HRMS (ESI) m/z calcd for C₂₀H₁₉N₅O [M+H]⁺ 346.1667, found 346.1655.

### (3-(1H-pyrrolo[2,3-b]pyridin-5-yl)pyrazolo[1,5-a]pyridin-5-yl)(piperazin-1-yl)methanone (CDD-2487)

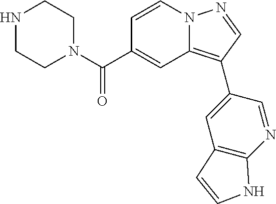 [SMILES: O=C(c1ccn2ncc(-c3cnc4[nH]ccc4c3)c2c1)N1CCNCC1]

Yield 65% (87 mg); White solid ¹H NMR (600 MHz, DMSO) δ 11.81 (s, 1H), 9.12 (s, 2H). 8.82 (d, J=7.1 Hz, 1H). 8.60-8.54 (m, 1H), 8.48 (s, 1H), 8.27 (d. J=2.0 Hz, 1H), 8.08 (s. 1H), 7.54 (t, J=2.9 Hz, 1H), 6.99 (d, J=7.1 Hz, 1H), 6.56-6.47 (m, 1H), 3.77 (s, 4H), 3.19 (s, 4H); ¹³C NMR (151 MHz, DMSO) δ 167.93, 147.44, 141.58, 135.60, 131.22, 129.96, 127.54, 126.97, 120.66, 120.62, 116.71, 112.40, 111.50, 100.53, 49.06, 42.98; HRMS (ESI) m/z calcd for C₁₉H₁₉N₆O [M+H]⁺ 347.162, found 347.1611.

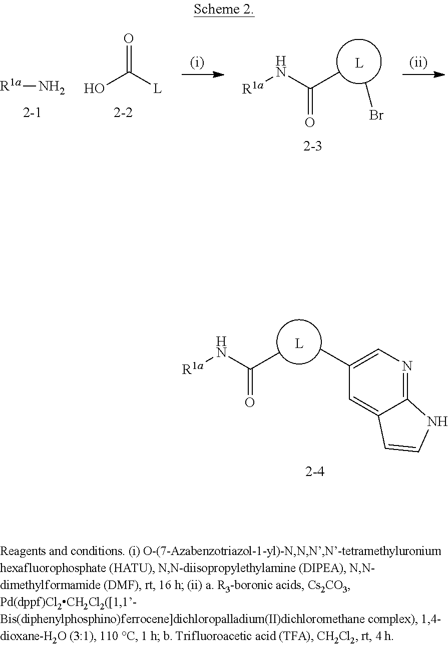

### General Procedure for Amide Synthesis (i.e., Step (i) in Scheme 2)

To a stirred solution of substituted amines (100 mg. 0.4 mmol, 1.0 eq) in anhydrous DMF (2 mL) was added substituted acids (1.0 eq), HATU (193 mg, 0.5 mmol, 1.2 eq), DIPEA (222 μL, 1.27 mmol, 3.0 eq), and the resulting mixture was stirred at room temperature for 14 h. After completion, the reaction (monitored by TLC and LC-MS) was quenched by adding water and extracted with 20 mL of ethyl acetate (EtOAc) thrice. The combined organic layers were washed with brine solution (NaCl), dried over anhydrous sodium sulfate (Na₂SO₄), filtered, and concentrated under reduced pressure to give a crude reaction mixture. It was purified by silica gel column chromatography (Teledyne ISCO CombiFlash system) using hexane-EtOAc (100-0 to 0-100) to afford the desired compound in moderate to good yield (69-90%).

### General Procedure for Suzuki Cross-Coupling Reaction (i.e., Step (ii) in Scheme 2)

A 10 mL oven dried microwave vial equipped with magnetic stir bar was charged with the 1.0 eq of bromo-intermediates (2s-2y), (1H-pyrrolo[2,3-b]pyridin-5-yl)boronic acid (1.2 eq), Cs₂CO₃ (2.5 eq), and Pd(dppf)Cl₂·CH₂Cl₂ (0.1 eq) followed by addition of 1,4-dioxane-H₂O (3:1, 4 mL). The composition of a vial was sealed with a microwave cap, purged with nitrogen for 25 mins, and then irradiated the vial at 110° C. under microwave conditions for 1 h. After completing the reaction (monitored by TLC and LC-MS), the crude material containing unwanted catalyst was removed by filtered using celite. The clear solution was extracted with 20 mL of ethyl acetate (EtOAc) thrice, the combined organic layers were washed with brine solution (NaCl), and dried over anhydrous sodium sulfate (Na₂SO₄), filtered, and concentered under reduced pressure to give a crude reaction mixture. It was purified by normal phased silica gel column chromatography (Teledyne ISCO CombiFlash system) CH₂Cl₂-MeOH (100-0 to 0-10) as eluent to afford the Boc-protected compounds. Further, it was reacted with TFA in CH₂Cl₂ to remove the Boc protection and purified by reversed phase column chromatography (Teledyne ISCO CombiFlash system) using solvent CH₃CN—H₂O (0-90 to 95-5) to obtain the desired compound in 46-82% yield.

The following exemplary compounds were prepared according to the synthesis depicted Scheme 2.

### 1-(5-(1H-pyrrolo[2,3-b]pyridin-5-yl)pyridin-3-yl)-N-(4 ((methylamino)methyl)phenyl)cyclopropane-1-carboxamide (CDD-2519)

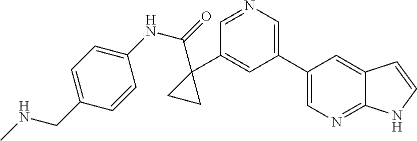 [SMILES: CNCc1ccc(NC(=O)C2(c3cncc(-c4cnc5[nH]ccc5c4)c3)CC2)cc1]

Yield 60% (80 mg); White solid ¹H NMR (600 MHz, DMSO) δ 11.82 (s, 1H). 9.37 (s, 1H), 8.88 (s, 1H), 8.78 (s, 1H), 8.63-8.59 (m, 2H), 8.35 (d, J=2.1 Hz, 1H), 8.12 (t, J=2.2 Hz, 1H), 7.62 (d, J=8.6 Hz, 2H), 7.56 (t, J=2.9 Hz, 1H), 7.37 (d, J=8.4 Hz, 2H), 6.54 (dd, J=3.4, 1.8 Hz, 1H), 4.05 (s, 2H), 2.53 (s, 3H), 1.56 (q. J=4.3 Hz, 2H), 1.33 (q. J=4.4 Hz. 2H); ¹³C NMR (151 MHz, DMSO) δ 171.28, 149.62, 148.81, 146.71, 142.01, 139.93, 136.28, 135.57, 134.83, 130.50, 127.73, 127.46, 127.02, 125.31, 121.22, 120.22, 100.74, 51.43, 32.44, 29.87, 15.27: HRMS (ESI) m/z calcd for C₂₄H₂₄N₅O [M+H]⁺ 398.1980, found 398.1969.

### N-(4-((methylamino)methyl)phenyl)-4-(1H-pyrrolo[2,3-b]pyridin-5-yl)benzamide (CDD-2520)

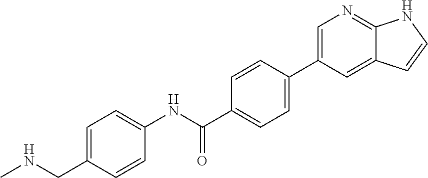 [SMILES: CNCc1ccc(NC(=O)c2ccc(-c3cnc4[nH]ccc4c3)cc2)cc1]

Yield 46% (91 mg); White solid; ¹H NMR (600 MHz, DMSO) δ 11.91 (s, 1H), 10.45 (s, 1H), 8.88-8.82 (m, 2H). 8.66 (d, J=2.3 Hz, 1H). 8.39 (d, J=2.2 Hz, 1H). 8.11 (d, J=8.2 Hz, 2H), 7.93 (d, J=8.1 Hz, 2H), 7.89 (d, J=8.2 Hz, 2H), 7.58 (t, J=2.9 Hz, 1H), 7.48 (d, J 8.3 Hz, 2H), 6.58 (dd, J=3.5, 1.8 Hz, 1H), 4.12 (t, J 5.9 Hz, 2H), 2.58 (t, J=5.4 Hz. 3H); ¹³C NMR (151 MHz, DMSO) δ 165.80, 148.21, 142.60, 141.48, 140.40, 133.37, 130.82, 128.93, 127.96, 127.52, 127.44, 127.31, 127.14, 120.83, 120.62, 100.98, 51.48, 32.45; HRMS (ESI) m/z calcd for C₂₂H₂₁N₄O [M+H]⁺ 357.1715, found 357.1705.

### N-(4-((methylamino)methyl)phenyl)-3-(1H-pyrrolo[2,3-b]pyridin-5-yl)imidazo[1,2-a]pyridine-6-carboxamide (CDD-2488)

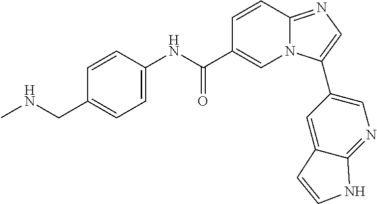 [SMILES: CNCc1ccc(NC(=O)c2ccc3ncc(-c4cnc5[nH]ccc5c4)n3c2)cc1]

Yield 63% (87 mg); White solid, ¹H NMR (600 MHz, DMSO) δ 12.08 (s, 1H), 10.74 (s, 1H), 9.21 (s, 1H). 8.94 (d, J=5.3 Hz, 1H). 8.57 (d, J=2.1 Hz, 1H). 8.43 (d, J=2.1 Hz, 1H), 8.34 (s, 1H), 8.26 (dd, J=9.4, 1.6 Hz, 1H), 8.10 (d, J=9.4 Hz, 1H), 7.76 (d, J=8.5 Hz, 2H), 7.67 (t, J 3.0 Hz, 1H), 7.47 (d, J 8.4 Hz, 2H), 6.63 (dd, J=3.4, 1.8 Hz, 1H), 4.11 (t, J=5.8 Hz, 2H), 2.56 (t, J=5.3 Hz, 3H); ¹³C NMR (151 MHz, DMSO) δ 162.90, 149.16, 143.54, 142.53, 139.58, 130.90, 129.62, 128.37, 128.02, 127.10, 126.72, 123.75, 121.21, 120.14, 117.92, 115.96, 114.55, 114.39, 101.03, 51.35, 32.42: HRMS (ESI) m/z calcd for C₂₃H₂₁N₆O [M+H]⁺ 397.1776, found 397.1772.

### N-(4-((methylamino)methyl)phenyl)-4-(1H-pyrrolo[2,3-b]pyridin-5-yl)benzo[b]thiophene-2-carboxamide (CDD-2575)

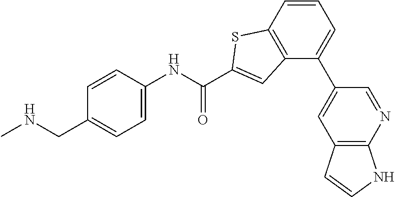 [SMILES: CNCc1ccc(NC(=O)c2cc3c(-c4cnc5[nH]ccc5c4)cccc3s2)cc1]

Yield 82% (46 mg); Light brown solid; ¹H NMR (600 MHz, MeOD) δ 8.42-8.39 (m, 1H), 8.26 (s, 1H), 8.21 (d, J=2.0 Hz, 1H), 7.93 (d, J=8.1 Hz, 1H), 7.62 (d, J=8.4 Hz, 2H), 7.54 (t, J=7.7 Hz, 1H), 7.49 (d, J=3.5 Hz, 1H), 7.44 (d, J=7.3 Hz, 1H), 7.29 (d, J=8.4 Hz, 2H), 6.61 (d, J=3.5 Hz, 1H), 3.67 (s, 2H), 2.37 (s, 3H); ¹³C NMR (151 MHz, MeOD) δ 161.54, 147.52, 142.24, 142.13, 139.71, 138.25, 137.40, 137.24, 134.98, 129.24, 128.64, 128.29, 126.61, 126.43, 125.56, 124.49, 121.34, 120.78, 120.68, 100.51, 54.38, 33.99; HRMS (ESI) m/z calcd for C₂₄H₂₁N₄OS [M+H]⁺ 413.1436, found 413.1435.

### N-(3-(dimethylcarbamoyl)-4-hydroxyphenyl)-4-(1H-pyrrolo[2,3-b]pyridin-5-yl)benzofuran-2-carboxamide (CDD-2855)

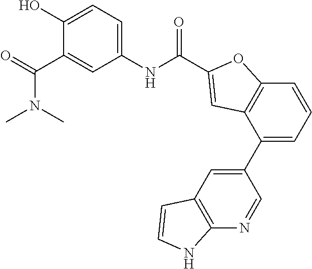 [SMILES: CN(C)C(=O)c1cc(NC(=O)c2cc3c(-c4cnc5[nH]ccc5c4)cccc3o2)ccc1O]

Yield 45% (43 mg); white solid; ¹H NMR (600 MHz, DMSO) δ 11.87 (t, J=2.2 Hz, 1H), 10.38 (s, 1H), 9.77 (s, 1H), 8.55 (d, J=2.2 Hz, 1H), 8.28 (d, J=2.1 Hz, 1H), 7.97 (d, J=1.0 Hz, 1H), 7.73 (dt, J=8.3, 1.0 Hz, 1H), 7.63-7.59 (m, 3H), 7.57 (d, J=2.7 Hz, 1H), 7.53 (dd, J=7.4, 0.9 Hz, 1H), 6.88 (d. J=8.8 Hz, 1H), 6.59 (dd, J=3.4, 1.8 Hz. 1H), 2.97 (s, 3H), 2.86 (s, 3H): ¹³C NMR 13C NMR (151 MHz, DMSO) δ 168.59, 156.67, 155.43, 150.25, 149.61, 148.62, 142.87, 135.19, 130.57, 128.12, 127.92, 127.72, 126.97, 126.08, 124.80, 123.74, 123.01, 120.73, 120.19, 116.24, 111.05, 109.64, 100.84, 38.08, 34.65; HRMS (ESI) m/z calcd for C₂₅H₂₁N₄O₄ [M+H]⁺ 441.1562, found 441.1550.

### N-(3-(dimethylcarbamoyl)-4-hydroxyphenyl)-4-(1H-pyrrolo[2,3-b]pyridin-5-yl)benzo[d]thiazole-2-carboxamide (CDD-2856)

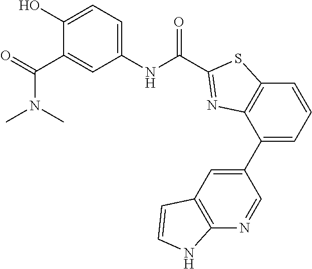 [SMILES: CN(C)C(=O)c1cc(NC(=O)c2nc3c(-c4cnc5[nH]ccc5c4)cccc3s2)ccc1O]

Yield 49% (28 mg); white solid; ¹H NMR (600 MHz, DMSO) δ 11.77 (s, 1H), 10.58 (s, 1H), 9.85 (s, 1H), 8.77 (d, J=2.1 Hz, 1H), 8.53 (d, J=2.2 Hz, 1H), 8.26-8.21 (m. 1H), 7.80 (dd, J=7.5, 1.1 Hz, 1H), 7.70 (t, J=7.7 Hz, 1H), 7.64 (dd, J=8.8, 2.7 Hz, 1H), 7.59-7.53 (m, 2H), 6.89 (d, J=8.8 Hz, 1H), 6.59 (dd, J=3.4, 1.8 Hz, 1H), 2.97 (s, 3H), 2.85 (s, 3H); 13C NMR (151 MHz, DMSO) δ 168.48, 164.56, 158.50, 150.83, 150.75, 148.54, 144.27, 137.88, 136.14, 132.96, 129.74, 129.70, 127.85, 127.66, 127.16, 126.33, 124.83, 123.77, 122.00, 121.53, 119.90, 116.20, 101.03, 38.06, 34.62: HRMS (ESI) m/z calcd for C₂₄H₂₀N₅O₃S [M+H]⁺ 458.1286, found 458.1280.

### N-(3-(dimethylcarbamoyl)-4-hydroxyphenyl)-4-(1H-pyrrolo[2,3-b]pyridin-5-yl)benzo[d]thiazole-2-carboxamide (CDD-3016)

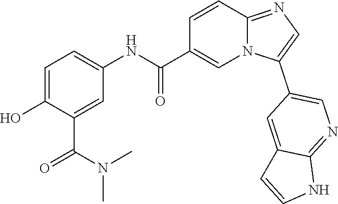 [SMILES: CN(C)C(=O)c1cc(NC(=O)c2ccc3ncc(-c4cnc5[nH]ccc5c4)n3c2)ccc1O]

Yield 36% (14 mg); white solid; 1H NMR (600 MHz, DMSO) δ 11.94 (s, 1H), 10.23 (s, 1H), 9.08 (s, 1H). 8.54-8.51 (m, 1H), 8.35-8.32 (m. 1H), 7.87 (s, 1H), 7.78 (q, J=9.5 Hz, 3H), 7.61 (d, J=3.5 Hz, 1H), 7.51 (dd, J=8.8, 2.7 Hz, 1H), 7.45 (d, J=2.7 Hz, 1H), 6.85 (d, J=8.7 Hz, 1H), 6.59 (d, J=3.4 Hz, 1H), 2.95 (s, 3H), 2.85 (s, 3H); HRMS (ESI) m/z calcd for C₂₄H₂₁N₆O₃ [M+H]⁺ 441.1675, found 441.1669.

### N-(3-(dimethylcarbamoyl)-4-hydroxyphenyl)-4-(1H-pyrrolo[2,3-b]pyridin-5-yl)thieno[2,3-c]pyridine-2-carboxamide (CDD-3111)

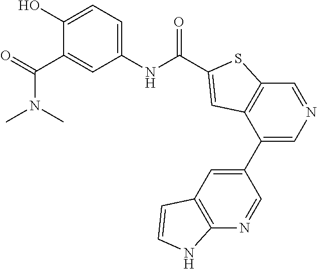 [SMILES: CN(C)C(=O)c1cc(NC(=O)c2cc3c(-c4cnc5[nH]ccc5c4)cncc3s2)ccc1O]

Yield 29% (21 mg); white solid; 1H NMR (600 MHz, DMSO) δ 11.89 (d, J=2.3 Hz, 1H), 10.53 (s, 1H), 9.74 (s, 1H), 9.38 (s, 1H), 8.60 (s, 1H), 8.48-8.45 (m, 2H), 8.24 (d, J=2.1 Hz, 1H), 7.58-7.54 (m, 1H), 7.47 (dd, J=8.8, 2.7 Hz, 1H), 7.39 (d, J=2.6 Hz, 1H), 6.80 (d, J=8.8 Hz, 1H), 6.55 (dd, J=3.5, 1.8 Hz, 1H), 2.82 (d, J=72.6 Hz, 6H); ¹³C NMR (151 MHz, DMSO) δ 168.48, 159.40, 150.56, 148.81, 145.01, 143.18, 142.92, 141.01, 137.30, 132.54, 130.14, 129.29, 128.05, 124.90, 124.09, 123.51, 123.29, 121.12, 120.21, 116.27, 100.98, 34.68; HRMS (ESI) m/z calcd for C₂₄H₂₀N₅O₃S [M+H]⁺ 458.1286, found 458.1283.

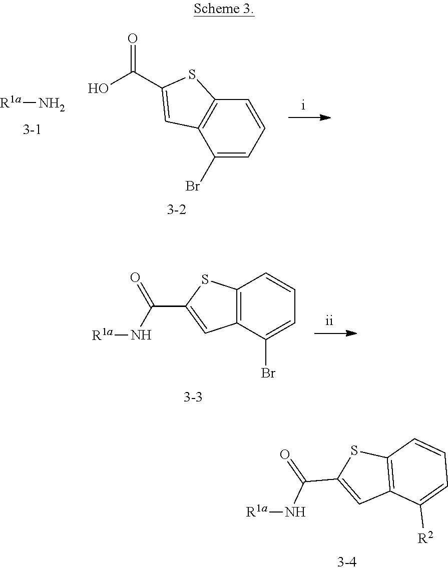

### General Procedure for Amide Synthesis (i.e., Step (i) in Scheme 3)

To a stirred solution of substituted amines (1.0 eq) in anhydrous DMF (2 mL) was added substituted acids (1.0 eq), HATU (1.2 eq), DIPEA (3.0 eq), and the resulting mixture was stirred at room temperature for 16 h. After completion, the reaction (monitored by TLC and LC-MS) was quenched by adding water and extracted with 20 mL of ethyl acetate (EtOAc) thrice. The combined organic layers were washed with brine solution (NaCl), dried over anhydrous sodium sulfate (Na₂SO₄), filtered, and concentrated under reduced pressure to give a crude reaction mixture. It was purified by silica gel column chromatography (Teledyne ISCO CombiFlash system) using CH₂Cl₂-MeOH (100-0 to 0-10) to afford the desired compound in moderate to good yield (72-84%).

### General Procedure for Suzuki Cross-Coupling Reaction (i.e., Step (ii) in Scheme 3)

A 10 mL oven dried microwave vial equipped with magnetic stir bar was charged with the 1.0 eq of aryl halide intermediates, boronic acid (1.2 eq). Cs₂CO₃ (2.5 eq), and Pd(dppf)Cl₂·CH₂Cl₂ (0.1 eq) followed by addition of 1,4-dioxane-H₂O (3:1, 4 mL). The composition of a vial was sealed with a microwave cap, purged with nitrogen for 25 mins, and then irradiated the vial at 110° C. under microwave conditions for 1 h. After completing the reaction (monitored by TLC and LC-MS), the crude material containing unwanted catalyst was removed by filtered using celite. The clear solution was extracted with 20 mL of ethyl acetate (EtOAc) thrice, the combined organic layers were washed with brine solution (NaCl), and dried over anhydrous sodium sulfate (Na₂SO₄), filtered, and concentered under reduced pressure to give a crude reaction mixture. It was purified by normal phased silica gel column chromatography (Teledyne ISCO CombiFlash system) CH₂Cl₂-MeOH (100-0 to 0-10) as eluent to afford the desired compounds in 35-72% yield.

The following exemplary compounds were prepared according to the synthesis depicted Scheme 3.

### N-methyl-4-(1H-pyrrolo[2,3-b]pyridin-5-yl)benzo[b]thiophene-2-carboxamide (CDD-2627)

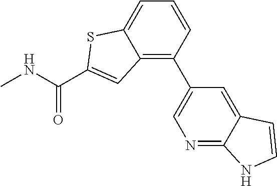 [SMILES: CNC(=O)c1cc2c(-c3cnc4[nH]ccc4c3)cccc2s1]

Yield 73% (53 mg); Light brown solid; ¹H NMR (600 MHz, DMSO) δ 11.84 (s, 1H), 8.78 (d, J=5.1 Hz, 1H), 8.42 (s. 1H), 8.14 (d. J=8.8 Hz. 2H), 8.03 (d. J=8.1 Hz. 1H), 7.61-7.52 (m, 2H), 7.46 (d, J=7.2 Hz, 1H), 6.59-6.56 (m, 1H), 2.76 (d, J=4.7 Hz, 3H); ¹³C NMR (151 MHz, DMSO) δ 162.31, 148.51, 143.27, 141.40, 140.82, 138.44, 137.55, 128.75, 128.01, 127.55, 126.77, 126.06, 123.80, 122.13, 119.98, 100.78, 26.51; HRMS (ESI) m/z calcd for C₁₇H₁₄N₃OS [M+H]⁺ 308.0857, found 308.0853.

### N-phenyl-4-(1H-pyrrolo[2, 3-b]pyridin-5-yl)benzo[b]thiophene-2-carboxamide (CDD-2631)

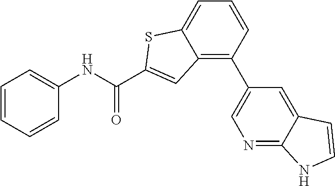 [SMILES: O=C(Nc1ccccc1)c1cc2c(-c3cnc4[nH]ccc4c3)cccc2s1]

Yield 64% (72 mg); White solid; ¹H NMR (600 MHz, DMSO) δ 11.87 (s, 1H), 10.53 (s, 1H). 8.47 (d, J=8.2 Hz, 2H). 8.20 (d, J=2.1 Hz, 1H). 8.08 (d, J=8.1 Hz, 1H). 7.71 (d, J=8.0 Hz, 2H), 7.63-7.57 (m, 2H), 7.50 (d, J=7.3 Hz, 1H), 7.36 (t, J=7.7 Hz, 2H), 7.12 (t, J=7.4 Hz, 1H), 6.59 (dd, J=3.4, 1.8 Hz, 1H); ¹³C NMR (151 MHz, DMSO) δ 160.73, 148.56, 143.31, 141.81, 140.85, 138.93, 138.45, 137.95, 129.15, 128.84, 128.06, 127.61, 127.18, 126.31, 125.16, 124.47, 122.16, 121.05, 120.08, 100.83; HRMS (ESI) m/z calcd for C₂₂H₁₆N₃OS [M+H]⁺ 370.1014, found 370.1009.

### N-(4-methoxyphenyl)-4-(1H-pyrrolo[2,3-b]pyridin-5-yl)benzo[b]thiophene-2-carboxamide (6 CDD-2629)

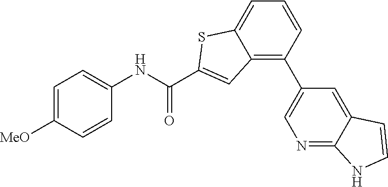 [SMILES: COc1ccc(NC(=O)c2cc3c(-c4cnc5[nH]ccc5c4)cccc3s2)cc1]

Yield 72% (48 mg); White solid ¹H NMR (600 MHz, DMSO) δ 11.86 (s, 1H), 10.43 (s, 1H), 8.47 (d, J=2.1 Hz, 1H), 8.42 (s, 1H), 8.20 (d, J=2.2 Hz, 1H), 8.07 (d, J=8.1 Hz, 1H), 7.63-7.57 (m, 4H), 7.49 (dd, J=7.2, 1.1 Hz, 1H), 6.95-6.90 (m, 2H), 6.59 (dd. J=3.4, 1.8 Hz, 1H). 3.75 (s. 3H); ¹³C NMR (151 MHz, DMSO) δ 160.36, 156.26, 148.55, 143.31, 141.71, 141.06, 138.49, 137.85, 131.91, 128.83, 128.08, 127.60, 127.06, 126.27, 124.75, 122.67, 122.15, 120.07, 114.30, 100.83, 55.66; HRMS (ESI) m/z calcd for C₂₃H₁₈N₃O₂S [M+H]⁺ 400.1119, found 400.1101.

### N-(4-methoxy-3-(methylcarbonyl)phenyl)-4-(1H-pyrrolo[2,3-b]pyridin-5-yl)benzo[b]thiophene-2-carboxamide (CDD-2490)

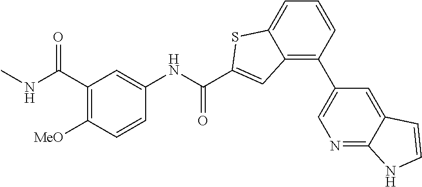 [SMILES: CNC(=O)c1cc(NC(=O)c2cc3c(-c4cnc5[nH]ccc5c4)cccc3s2)ccc1OC]

Yield 70% (113 mg): White solid; ¹H NMR (600 MHz, DMSO) δ 11.79 (s. 1H), 10.48 (s, 11H), 8.40 (d, J=5.5 Hz, 2H), 8.13 (s, 2H), 7.99 (t, J=6.4 Hz, 2H), 7.89-7.84 (m, 1H), 7.52 (dd, J=9.9, 5.6 Hz, 2H), 7.42 (d, J=7.3 Hz, 1H), 7.06 (d, J=9.0 Hz, 1H), 6.54-6.50 (m, 1H), 3.81 (s, 3H), 2.74 (d, J=4.4 Hz, 3H); ¹³C NMR (151 MHz, DMSO) δ 165.38, 160.49, 153.88, 148.57, 143.31, 141.79, 140.79, 138.49, 137.91, 132.04, 128.84, 128.02, 127.59, 127.14, 126.27, 125.06, 124.69, 123.41, 123.09, 122.15, 120.07, 112.71, 100.83, 56.53, 26.86; HRMS (ESI) m/z calcd for C₂₅H₂₁N₄O₃S [M+H]⁺ 457.1334, found 457.1321.

### N-(3-(dimethylcarbamoyl)-4-methoxyphenyl)-4-(1H-pyrrolo[2,3-b]pyridin-5-yl)benzo[b]thiophene-2-carboxamide (CDD-2628)

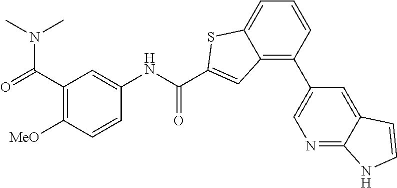 [SMILES: COc1ccc(NC(=O)c2cc3c(-c4cnc5[nH]ccc5c4)cccc3s2)cc1C(=O)N(C)C]

Yield 74% (146 mg); Light yellow solid: ¹H NMR (600 MHz, DMSO) δ 11.87 (s, 1H), 10.50 (s, 1H), 8.48 (d, J=2.1 Hz, 1H), 8.44 (s, 1H), 8.21 (d, J=2.1 Hz, 1H), 8.09 (d. J=8.1 Hz. 1H), 7.72 (dd, J=9.0, 2.7 Hz. 1H), 7.64-7.58 (m, 2H), 7.55-7.49 (m, 2H), 7.10 (d, J=9.0 Hz, 1H), 6.60 (dd, J=3.4, 1.8 Hz, 1H), 3.80 (s, 3H), 2.98 (s, 3H), 2.79 (s, 3H); ¹³C NMR (151 MHz, DMSO) δ 167.96, 160.44, 151.75, 148.56, 143.30, 141.77, 140.79, 138.46, 137.90, 132.04, 128.83, 128.03, 127.61, 127.14, 126.64, 126.30, 124.93, 122.73, 122.17, 120.55, 120.06, 112.15, 100.83, 56.21, 38.01, 34.54; HRMS (ESI) m/z calcd for C₂₆H₂₃N₄O₃S [M+H]⁺ 471.149, found 471.1481.

### N-(4-methoxy-3-(piperidine-1-carbonyl)phenyl)-4-(1H-pyrrolo[2,3-b]pyridin-5-yl)benzo[b]thiophene-2-carboxamide (CDD-2491)

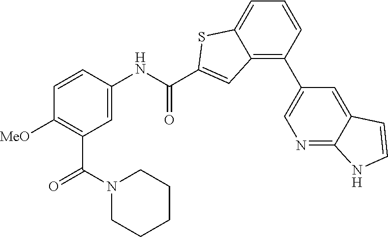 [SMILES: COc1ccc(NC(=O)c2cc3c(-c4cnc5[nH]ccc5c4)cccc3s2)cc1C(=O)N1CCCCC1]

Yield 60% (98 mg); White solid, ¹H NMR (600 MHz, DMSO) δ 11.85 (s, 1H). 10.47 (s, 1H), 8.45 (d, J=2.1 Hz, 1H), 8.42 (s, 1H), 8.19 (d. J=2.1 Hz, 1H), 8.06 (d, J=8.1 Hz, 1H), 7.70 (dd, J=8.9, 2.6 Hz, 1H), 7.61-7.55 (m, 2H), 7.52-7.46 (m, 2H), 7.06 (d, J=9.0 Hz, 1H), 6.58 (dd, J=3.4, 1.8 Hz, 1H), 3.77 (s, 3H), 3.67 (ddt, J=13.2, 6.7, 3.4 Hz, 1H), 3.46 (ddd, J=11.9, 7.6, 3.6 Hz, 1H), 3.35 (s, 3H), 1.61-1.31 (m, 6H): ¹³C NMR (151 MHz, DMSO) δ 166.19, 160.44, 151.73, 148.56, 143.30, 141.77, 140.79, 138.46, 137.90, 132.07. 128.83, 128.04, 127.61, 127.14, 126.56, 126.30, 124.93, 122.58, 122.16, 120.39, 120.07, 112.06, 100.83, 56.17, 47.66, 42.19, 26.43, 25.82, 24.49; HRMS (ESI) m/z calcd for C₂₉H₂₇N₄O₃S [M+H]⁺ 511.1803. found 511.1792.

### N-(4-methoxy-3-(morpholine-4-carbonyl)phenyl)-4-(1H-pyrrolo[2,3-b]pyridin-5-yl)benzo[b]thiophene-2-carboxamide (CDD-2635)

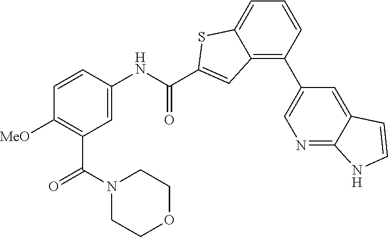 [SMILES: COc1ccc(NC(=O)c2cc3c(-c4cnc5[nH]ccc5c4)cccc3s2)cc1C(=O)N1CCOCC1]

Yield 68% (138 mg); White solid; ¹H NMR (600 MHz, DMSO) δ 11.86 (s, 1H), 10.51 (s, 1H), 8.46 (d, J=2.2 Hz, 1H), 8.43 (s, 1H), 8.20 (d, J=2.2 Hz, 1H), 8.07 (d, J=8.1 Hz, 1H), 7.73 (dd, J=9.0, 2.6 Hz, 1H), 7.62-7.57 (m, 2H), 7.55 (d, J=2.6 Hz, 1H), 7.49 (d, J=7.3 Hz, 1H), 7.09 (d, J=9.0 Hz, 1H), 6.59 (dd, J=3.4, 1.8 Hz, 1H), 3.80 (s, 3H), 3.62 (d, J=4.2 Hz. 4H), 3.56-3.45 (m. 2H), 3.16 (dq, J=14.3, 5.2 Hz, 2H): ¹³C NMR (151 MHz, DMSO) δ 166.54, 160.45, 151.75, 148.56, 143.29, 141.77, 140.75, 138.45, 137.90, 132.14, 128.83, 128.03, 127.61, 127.15, 126.30, 125.58, 124.97, 123.03, 122.17, 120.79, 120.06, 112.13, 100.83, 66.78, 66.55, 56.21, 47.26, 42.09: HRMS (ESI) m/z calcd for C₂₈H₂₅N₄O₄S [M+H]⁺ 513.1596. found 513.1590.

### N-(4-methoxy-3-(4-methylpiperazine-1-carbonyl)phenyl)-4-(1H-pyrrolo[2,3-b]pyridin-5-yl)benzo[b]thiophene-2-carboxamide (CDD-2636)

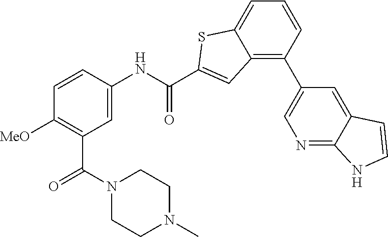 [SMILES: COc1ccc(NC(=O)c2cc3c(-c4cnc5[nH]ccc5c4)cccc3s2)cc1C(=O)N1CCN(C)CC1]

Yield 72% (126 mg): White solid; ¹H NMR (600 MHz, DMSO) δ 11.86 (s. 1H), 10.48 (s, 1H), 8.46 (d, J=2.1 Hz, 1H), 8.42 (s, 1H), 8.19 (d, J=2.1 Hz. 1H), 8.07 (d, J=8.1 Hz, 1H), 7.72 (dd, J=9.0, 2.6 Hz, 1H), 7.62-7.57 (m, 2H), 7.53-7.47 (m, 2H), 7.08 (d, J=9.1 Hz, 1H), 6.59 (dd, J=3.4, 1.8 Hz, 1H), 3.78 (s, 3H), 3.69 (d, J=13.2 Hz, 1H), 3.53 (d, J=10.1 Hz, 1H), 3.14 (q, J=4.1 Hz, 2H), 2.39 (dd, J=10.8, 5.3 Hz, 1H), 2.28 (tt, J=13.0. 6.6 Hz, 2H), 2.18 (s, 3H), 2.14 (p, J=4.8 Hz, 1H); ¹³C NMR (151 MHz, DMSO) δ 166.36, 160.44, 151.74, 148.56, 143.29, 141.77, 140.77, 138.45, 137.90, 132.10, 128.83, 128.03, 127.61, 127.15, 126.30, 126.02, 124.95, 122.82, 122.17, 120.58, 120.06, 112.14, 100.83, 56.19, 55.23, 54.78, 46.64, 46.11, 41.40: HRMS (ESI) m/z calcd for C₂₉H₂₈N₅O₃S [M+H]⁺ 526.1912, found 526.1914.

### N-(3-(dimethylcarbamoyl)phenyl)-4-(1H-pyrrolo[2, 3-b]pyridin-5-yl)benzo[b]thiophene-2-carboxamide (CDD-2632)

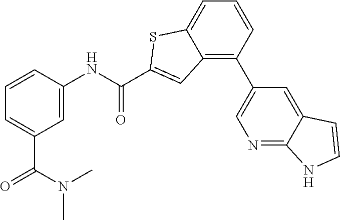 [SMILES: CN(C)C(=O)c1cccc(NC(=O)c2cc3c(-c4cnc5[nH]ccc5c4)cccc3s2)c1]

Yield 60% (78 mg); Light yellow solid; ¹H NMR (600 MHz, DMSO) δ 11.86 (s, 1H), 10.63 (s, 1H), 8.47 (d, J=1.8 Hz, 2H), 8.21 (d, J=2.1 Hz, 1H), 8.08 (d, J=8.1 Hz, 1H), 7.79-7.73 (m, 2H), 7.64-7.55 (m, 2H), 7.50 (d, 0.1=7.3 Hz, 1H), 7.41 (t, J=7.8 Hz, 1H), 7.14 (dt, J=7.6, 1.3 Hz, 1H), 6.59 (dd, J=3.4, 1.8 Hz, 1H), 2.96 (d, J=37.2 Hz, 6H).; ¹³C NMR (151 MHz, DMSO) δ 170.26, 160.89, 148.57, 143.29, 141.87, 140.53, 138.88, 138.41, 138.00, 137.39, 132.96, 129.19, 128.84, 128.01, 127.63, 127.28, 126.93, 126.35, 125.38, 122.80, 122.19, 121.63, 120.07, 119.43, 113.00, 100.83, 99.29, 49.07, 40.43, 40.29, 40.15, 40.02, 39.88, 39.74, 39.60, 35.20; HRMS (ESI) m/z calcd for C₂₅H₂₁N₄O₂S [M+H]⁺ 441.1385, found 441.1352.

### N-(3-(piperidine-1-carbonyl)phenyl)-4-(1H-pyrrolo[2,3-b]pyridin-5-yl)benzo[b]thiophene-2-carboxamide (CDD-2630)

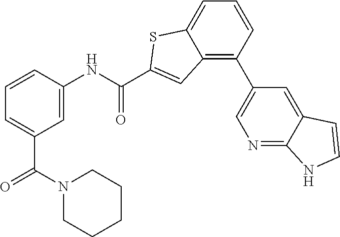 [SMILES: O=C(Nc1cccc(C(=O)N2CCCCC2)c1)c1cc2c(-c3cnc4[nH]ccc4c3)cccc2s1]

Yield 75% (163 mg); Light yellow solid; ¹H NMR (600 MHz, DMSO) δ 11.86 (s, 1H), 10.62 (s, 1H). 8.46 (s, 2H), 8.20 (s. 1H), 8.08 (d, J=8.1 Hz, 1H), 7.78-7.72 (m, 2H), 7.63-7.58 (m. 2H). 7.50 (d. J=7.3 Hz, 1H). 7.41 (t. J=7.8 Hz. 1H), 7.09 (d. J=7.5 Hz. 1H), 6.59 (d, J=3.2 Hz, 1H), 3.58 (s, 2H), 3.29 (s, 2H), 1.66-1.42 (m, 6H); ¹³C NMR (151 MHz, DMSO) δ 169.00, 160.89, 148.57, 143.29, 141.87, 140.52, 138.99, 138.40, 138.00, 137.44, 129.32, 128.84, 128.02, 127.63, 127.28, 126.35, 125.39, 122.46, 122.19, 121.49, 120.08, 119.01, 100.83, 48.48, 42.77, 26.50, 24.48; HRMS (ESI) m/z calcd for C₂₈H₂₅N₄O₂S [M+H]⁺ 481.1698, found 481.1691.

### N-(4-hydroxy-3-(piperidine-1-carbonyl)phenyl)-4-(1H-pyrrolo[2,3-b]pyridin-5-yl)benzo[b]thiophene-2-carboxamide (CDD-2634)

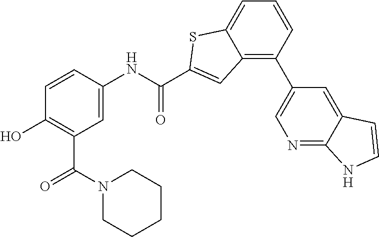 [SMILES: O=C(Nc1ccc(O)c(C(=O)N2CCCCC2)c1)c1cc2c(-c3cnc4[nH]ccc4c3)cccc2s1]

Yield 61% (16 mg); White solid ¹H NMR (600 MHz, DMSO) δ 11.85 (s, 1H), 10.39 (s, 1H), 9.66 (s, 1H), 8.43 (d, J=32.6 Hz, 2H), 8.19 (s, 1H), 8.06 (d, J=8.1 Hz, 1H), 7.60-7.56 (m, 2H), 7.54-7.48 (m, 2H). 7.45-7.42 (m, 1H). 6.83 (d, J=8.8 Hz, 1H). 6.59 (s. 1H). 3.51 (s, 2H), 3.17 (s, 2H), 1.64-1.39 (m, 6H); ¹³C NMR (151 MHz, DMSO) δ 166.83, 160.27, 150.16, 148.55, 143.29, 141.71, 140.97, 138.47, 137.84, 130.60, 129.01, 128.83, 128.04, 127.60, 127.07, 126.26, 124.74, 122.98, 122.15, 120.80, 120.05, 116.09, 100.82, 26.11, 24.52: HRMS (ESI) m/z calcd for C₂₈H₂₅N₄O₃S [M+H]⁺ 497.1647, found 497.1636.

### N-(3-(dimethylcarbamoyl)-4-hydroxyphenyl)-4-(1H-pyrrolo[2,3-b]pyridin-5-yl)benzo[b]thiophene-2-carboxamide (CDD-2728)

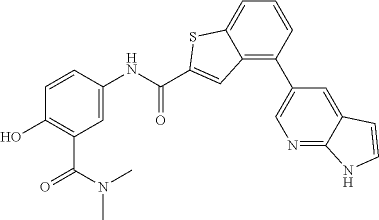 [SMILES: CN(C)C(=O)c1cc(NC(=O)c2cc3c(-c4cnc5[nH]ccc5c4)cccc3s2)ccc1O]

Yield 35% (27 mg): White solid: ¹H NMR (600 MHz, DMSO) δ 11.85 (s, 1H), 10.40 (s, 1H), 9.74 (s, 1H), 8.46 (d, J=2.1 Hz, 1H), 8.40 (s, 1H), 8.19 (d, J=2.1 Hz, 1H), 8.07 (d, J=8.1 Hz, 1H), 7.62-7.57 (m, 2H), 7.57-7.47 (m, 2H), 7.45 (d, J=2.6 Hz, 1H), 6.85 (d, J=8.8 Hz. 1H), 6.59 (dd, J=3.4, 1.7 Hz, 1H), 2.90 (d, J=65.4 Hz, 6H); ¹³C NMR (151 MHz, DMSO) δ 168.58, 160.29, 150.20, 148.54, 143.29, 141.71, 140.96, 138.46, 137.83, 130.58, 128.83, 128.03, 127.60, 127.08, 126.26, 124.78, 124.75, 123.18, 122.16, 120.96, 120.05, 116.17, 100.83, 40.54, 40.42, 40.28, 40.14, 40.01, 39.87, 39.73, 39.59; HRMS (ESI) m/z calcd for C₂₅H₂₁N₄O₃S [M+H]⁺ 457.1334, found 457.1320.

### N-(3-(dimethylcarbamoyl)-4-methylphenyl)-4-(1H-pyrrolo[2,3-b]pyridin-5-yl)benzo[b]thiophene-2-carboxamide (CDD-2729)

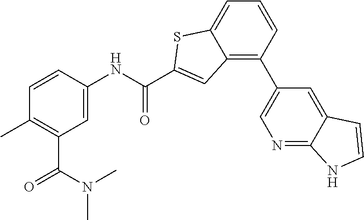 [SMILES: Cc1ccc(NC(=O)c2cc3c(-c4cnc5[nH]ccc5c4)cccc3s2)cc1C(=O)N(C)C]

Yield 58% (83 mg); White solid ¹H NMR (600 MHz, DMSO) δ 11.86 (s, 1H), 10.53 (s, 1H), 8.48-8.42 (m, 2H), 8.20 (d, J=2.1 Hz, 1H), 8.08 (d, J=8.1 Hz, 1H), 7.60 (qd, J=4.6, 3.0 Hz, 3H), 7.54 (d, J=2.2 Hz, 1H), 7.50 (d, J=7.3 Hz, 1H), 7.23 (d, J=8.3 Hz, 1H), 6.61-6.57 (m, 1H), 3.00 (s, 3H), 2.15 (s, 3H); ¹³C NMR (151 MHz, DMSO) δ 170.04, 160.68, 148.55, 143.29, 141.83, 140.68, 138.44, 137.95, 137.63, 136.73, 132.95, 130.88, 129.38, 128.84, 128.02, 127.63, 127.22, 126.94, 126.33, 125.15, 122.18, 120.90, 120.06, 118.07, 100.83, 38.20, 34.35, 18.37; HRMS (ESI) m/z calcd for C₂₆H₂₃N₄O₂S [M+H]⁺ 455.1541, found 455.1536.

### N-(3-(dimethylcarbamoyl)-4-(trifluoromethyl)phenyl)-4-(1H-pyrrolo[2,3-b]pyridin-5-yl)benzo[b]thiophene-2-carboxamide (CDD-2732)

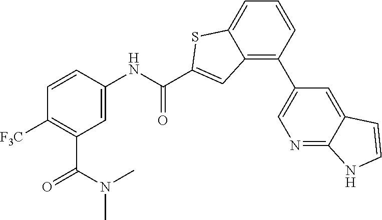 [SMILES: CN(C)C(=O)c1cc(NC(=O)c2cc3c(-c4cnc5[nH]ccc5c4)cccc3s2)ccc1C(F)(F)F]

Yield 42% (31 mg), White solid; ¹H NMR (600 MHz, DMSO) δ 11.87 (s, 1H), 10.87 (s, 1H), 8.51 (s, 1H), 8.47 (d, J=2.2 Hz, 1H), 8.20 (d, J=2.2 Hz, 1H), 8.10 (d, J=8.2 Hz, 1H), 7.94 (dd, J=8.5, 2.2 Hz, 1H), 7.80 (d, J=8.6 Hz, 2H), 7.65-7.59 (m, 2H), 7.51 (d, J=7.2 Hz, 1H), 6.59 (dd, J=3.4, 1.8 Hz, 1H), 3.00 (s, 3H), 2.75 (s, 3H); BC NMR (151 MHz, DMSO) δ 167.69, 161.31, 148.58, 143.28, 142.70, 142.07, 139.86, 138.35, 138.17, 136.63, 128.85, 128.02, 127.96, 127.68, 127.54, 126.45, 126.08, 125.16, 123.36, 122.24, 120.41, 120.08, 118.51, 100.84, 40.42, 40.28, 40.15, 40.01, 39.87, 39.73, 39.59, 38.69, 34.52; HRMS (ESI) m/z calcd for C₂₆H₂₀F3N₄O₂S [M+H]⁺ 509.1259, found 509.1250.

### N-(3-(dimethylcarbamoyl)-4-fluorophenyl)-4-(1H-pyrrolo[2,3-b]pyridin-5-yl)benzo[b]thiophene-2-carboxamide (CDD-2730)

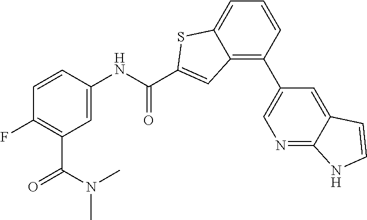 [SMILES: CN(C)C(=O)c1cc(NC(=O)c2cc3c(-c4cnc5[nH]ccc5c4)cccc3s2)ccc1F]

Yield 45% (63 mg); White solid ¹H NMR (600 MHz, DMSO) δ 11.86 (s, 1H), 10.65 (s, 1H), 8.47-8.42 (m, 2H), 8.19 (d, J=2.2 Hz, 1H), 8.08 (d, J=8.1 Hz, 1H), 7.79-7.70 (m, 2H), 7.63-7.58 (m, 2H), 7.50 (d, J=7.3 Hz, 1H), 7.29 (t, J=9.1 Hz, 1H), 6.60-6.56 (m, 1H), 3.00 (s, 3H), 2.86 (s, 3H); ¹³C NMR (151 MHz, DMSO) δ 165.50, 160.79, 154.91, 153.30, 148.56, 143.28, 141.86, 140.32, 138.38, 137.99, 135.53, 135.52, 128.84, 127.99, 127.65, 127.31, 126.36, 125.39, 125.12, 124.99, 123.53, 123.48, 40.43, 40.29, 40.15, 40.01, 39.87, 39.73, 39.59, 38.27, 34.76; HRMS (ESI) m/z calcd for C₂₅H₂₀FN₄O₂S [M+H]⁺ 459.1291, found 459.1288.

### N-(3-(dimethylcarbamoyl)-4-methoxyphenyl)-4-(3-methyl-1H-pyrrolo[2,3-b]pyridin-5-yl)benzo[b]thiophene-2-carboxamide (CDD-2731)

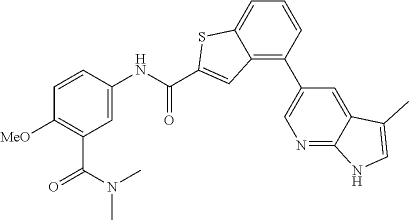 [SMILES: COc1ccc(NC(=O)c2cc3c(-c4cnc5[nH]cc(C)c5c4)cccc3s2)cc1C(=O)N(C)C]

Yield 55% (43 mg); Light yellow solid; ¹H NMR (600 MHz, DMSO) δ 11.51 (s, 1H), 10.49 (s, 1H), 8.44 (s, 2H), 8.13 (d, J=2.2 Hz, 1H), 8.07 (d, J=8.1 Hz, 1H), 7.70 (dd, J=9.0, 2.6 Hz, 1H), 7.59 (t, J=7.7 Hz, 1H), 7.51 (dd, J=8.0, 5.0 Hz, 2H), 7.36 (s, 1H), 7.09 (d, J=9.0 Hz, 1H), 3.79 (s, 3H), 2.97 (s, 3H), 2.77 (s, 3H), 2.34 (s, 3H); ¹³C NMR (151 MHz, DMSO) δ 167.95, 160.44, 151.75, 148.72, 143.10, 141.74, 140.75, 138.49, 138.08, 132.03, 127.33, 127.30, 127.12, 126.61, 126.32, 124.97, 124.74, 122.76, 122.10, 120.55, 120.51, 112.13, 109.41, 56.20, 38.01, 34.54, 10.18, 10.15; HRMS (ESI) m/z calcd for C₂₇H₂₅N₄O₃S [M+H]⁻ 485.1647, found 485.1638.

### N-(3-(dimethylcarbamoyl)-4-(trifluoromethoxy)phenyl)-4-(1H-pyrrolo[2, 3-b]pyridin-5-yl)benzo[b]thiophene-2-carboxamide (CDD-2857)

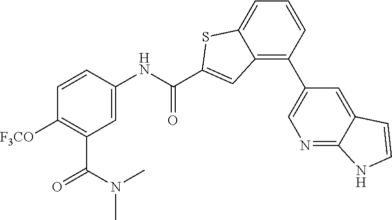 [SMILES: CN(C)C(=O)c1cc(NC(=O)c2cc3c(-c4cnc5[nH]ccc5c4)cccc3s2)ccc1OC(F)(F)F]

Yield 39% (24 mg); white solid; ¹H NMR (600 MHz, DMSO) δ 11.88 (s. 1H), 10.77 (s, 1H), 8.47 (d, J=4.2 Hz, 2H). 8.20 (s, 1H), 8.09 (d, J=8.2 Hz, 1H), 7.88-7.78 (m, 2H), 7.64-7.57 (m, 2H), 7.49 (dd, J=23.8, 8.2 Hz, 2H), 6.59 (s, 1H), 3.01 (s, 3H), 2.81 (s, 3H); ¹³C NMR (151 MHz, DMSO) δ 165.71, 161.00, 148.57, 143.28, 141.94, 140.10, 140.01, 138.36, 138.20, 138.06, 131.131, 128.84, 127.98, 127.66, 127.41, 126.40, 125.70, 122.62, 122.22, 122.20, 120.26, 120.08, 100.83, 38.27, 34.63: HRMS (ESI) m/z calcd for C₂₆H₂₀F3N₄O₃S [M+H]⁺ 525.1208, found 525.1194.

### N-(3-carbamoylphenyl)-4-(1H-pyrrolo[2, 3-b]pyridin-5-yl)benzo[b]thiophene-2-carboxamide (CDD-2936)

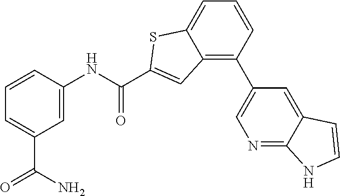 [SMILES: NC(=O)c1cccc(NC(=O)c2cc3c(-c4cnc5[nH]ccc5c4)cccc3s2)c1]

Yield 31% (35 mg); brown solid; ¹H NMR (600 MHz, DMSO) δ 11.86 (s, 1H), 10.66 (s, 1H), 8.51 (s, 1H), 8.47 (d, J=2.2 Hz, 1H), 8.21 (d, J=2.2 Hz, 1H), 8.15 (t, J=2.0 Hz, 1H), 8.09 (d. J=8.1 Hz. 1H), 7.97 (s, 1H), 7.93 (dd, J=8.0, 2.2 Hz, 1H), 7.61 (ddd, J=8.5, 6.4, 2.8 Hz, 3H), 7.50 (d, J=7.3 Hz, 1H), 7.43 (t, J=7.9 Hz, 1H), 7.37 (s, 1H), 6.60 (dd, J=3.4, 1.9 Hz, 1H); ¹³C NMR (151 MHz, DMSO) δ 168.17, 160.83, 148.58, 143.31, 141.88, 140.58, 138.97, 138.46, 138.01, 135.45, 129.03, 128.84, 128.01, 127.62, 127.26, 126.32, 125.40, 123.62, 123.19, 122.18, 120.61, 120.06, 100.83; HRMS (ESI) m/z calcd for C₂₃H₁₇N₄O₂S [M+H]⁺ 413.1072, found 413.1049.

### N-(3-(dimethylcarbamoyl)-5-hydroxyphenyl)-4-(H-pyrrolo[2,3-b]pyridin-5-yl)benzo[b]thiophene-2-carboxamide (CDD-2937)

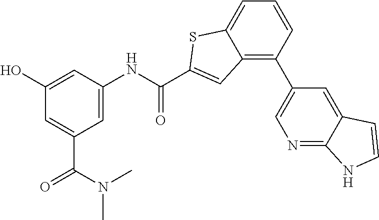 [SMILES: CN(C)C(=O)c1cc(O)cc(NC(=O)c2cc3c(-c4cnc5[nH]ccc5c4)cccc3s2)c1]

Yield 42% (35 mg); white solid; ¹H NMR (600 MHz, DMSO) δ 11.86 (s, 1H), 10.48 (s, 1H), 9.75 (s, 1H), 8.46 (d, J=6.9 Hz, 2H), 8.20 (d, J=2.2 Hz, 1H), 8.08 (d, J=8.1 Hz, 1H), 7.62-7.58 (m, 2H), 7.50 (d, J=7.2 Hz, 1H), 7.31 (t, J=2.1 Hz, 1H). 7.18-7.14 (m, 1H), 6.59 (dd, J=3.4, 1.8 Hz, 1H), 6.50 (t, J=1.8 Hz, 1H), 2.95 (s, 3H). 2.91 (s. 3H); ¹³C NMR (151 MHz, DMSO) δ 170.28, 160.77, 157.89, 148.56, 143.30, 141.86, 140.67, 139.89, 138.41, 138.25, 137.98, 128.86, 128.01, 127.63, 127.25, 126.33, 125.29, 122.17, 120.07, 110.15, 109.88, 108.54, 100.83, 40.55, 39.59, 35.09; HRMS (ESI) in z calcd for C₂₅H₂₁N₄O₃S [M+H]⁻ 457.1334, found 457.1316.

### N-(5-(dimethylcarbamoyl)-2-hydroxyphenyl)-4-(1H-pyrrolo[2,3-b]pyridin-5-yl)benzo[b]thiophene-2-carboxamide (CDD-2938)

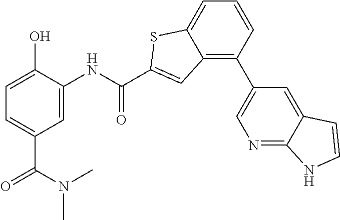 [SMILES: CN(C)C(=O)c1ccc(O)c(NC(=O)c2cc3c(-c4cnc5[nH]ccc5c4)cccc3s2)c1]

Yield 41% (35 mg); white solid; ¹H NMR (600 MHz, DMSO) δ 11.84 (s. 1H), 10.12 (d, J=7.1 Hz, 2H), 8.46 (d, J=9.7 Hz, 2H), 8.19 (s, 1H), 8.07 (d, J=8.1 Hz, 1H), 7.61-7.55 (m, 3H), 7.50 (d, J=7.3 Hz, 1H), 7.16 (d, J=8.3 Hz, 1H), 6.94 (d, J=8.3 Hz, 1H), 6.58 (s, 1H). 2.97 (s, 6H); ¹³C NMR (151 MHz, DMSO) δ 170.24, 161.03, 152.12, 148.53, 143.34, 141.73, 140.30, 138.40, 137.91, 131.18, 128.82, 127.99, 127.56, 127.16, 127.13, 126.44, 126.26, 125.98, 125.57, 124.65, 122.18, 120.00, 115.98, 100.79, 40.55, 35.34; HRMS (ESI) m/z calcd for C₂₅H₂₁N₄O₃S [M+H]⁺ 457.1334, found 457.1322.

### 4-(4-(1H-pyrrolo[2,3-b]pyridin-5-yl)benzo[b]thiophene-2-carboxamido)-N,N-dimethylpicolinamide (CDD-3012)

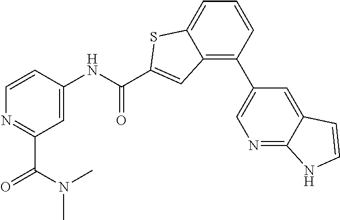 [SMILES: CN(C)C(=O)c1cc(NC(=O)c2cc3c(-c4cnc5[nH]ccc5c4)cccc3s2)ccn1]

Yield 50% (23 mg); white solid: ¹H NMR (600 MHz, DMSO) δ 11.88 (s. 1H), 10.94 (s, 1H), 8.53 (s, 1H), 8.47 (dd, J=10.9, 3.8 Hz, 2H), 8.21 (d, J=2.1 Hz, 1H), 8.10 (d, J=8.1 Hz, 1H), 7.89 (d, J=2.2 Hz, 1H), 7.80 (dd, J=5.5, 2.2 Hz, 1H), 7.66-7.59 (m, 2H), 7.52 (d, J=7.2 Hz, 1H), 6.61-6.57 (m, 1H), 3.01 (s, 3H), 2.96 (s, 3H): ¹³C NMR (151 MHz, DMSO) δ 168.37, 161.72, 155.84, 149.64, 148.59, 146.74, 143.27, 142.16, 139.58, 138.28, 138.25, 128.86, 127.92, 127.69, 127.66, 126.47, 122.25, 120.08, 114.80, 113.54, 100.84, 38.79, 35.38; HRMS (ESI) m/z calcd for C₂₄H₂₀N₅O₂S [M+H]⁺ 442.1337, found 442.1333.

### N-(4-hydroxy-3-(pyrrolidine-1-carbonyl)phenyl)-4-(1H-pyrrolo[2,3-b]pyridin-5-yl)benzo[b]thiophene-2-carboxamide (CDD-3013)

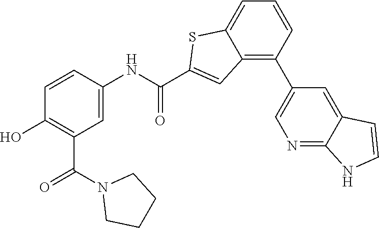 [SMILES: O=C(Nc1ccc(O)c(C(=O)N2CCCC2)c1)c1cc2c(-c3cnc4[nH]ccc4c3)cccc2s1]

Yield 46% (30 mg); brown solid; ¹H NMR (600 MHz, DMSO) δ 11.85 (s, 1H). 10.40 (s, 1H). 9.97 (s, 1H). 8.46 (d, J=2.0 Hz, 1H). 8.40 (s. 1H), 8.19 (d. J=2.1 Hz. 1H), 8.07 (d. J=8.0 Hz, 1H), 7.62-7.55 (m, 3H), 7.53 (dd, J=8.7, 2.6 Hz, 1H), 7.49 (d, =7.2 Hz, 1H), 6.86 (d, J=8.7 Hz, 1H), 6.61-6.57 (m. 1H), 3.44 (t, J=7.0 Hz, 2H), 3.33-3.28 (m, 2H), 1.89-1.78 (m. 4H).: ¹³C NMR (151 MHz, DMSO) δ 167.25, 160.31, 151.02, 148.55, 143.29, 141.71, 140.95, 138.46, 137.84, 130.39, 128.83, 128.04, 127.61, 127.09, 126.27, 124.75, 123.57, 122.16, 120.94, 120.05, 116.57, 100.83, 47.83, 45.96, 26.02, 24.46.; HRMS (ESI) m/z calcd for C₂₇H₂₃N₄O₃S [M+H]⁺ 483.1490, found 483.1483.

### N-(3-(azetidine-1-carbonyl)-4-hydroxyphenyl)-4-(1H-pyrrolo[2,3-b]pyridin-5-yl)benzo[b]thiophene-2-carboxamide (CDD-3014)

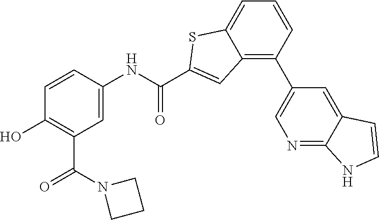 [SMILES: O=C(Nc1ccc(O)c(C(=O)N2CCC2)c1)c1cc2c(-c3cnc4[nH]ccc4c3)cccc2s1]

Yield 32% (3.5 mg); light brown solid; ¹H NMR (600 MHz, DMSO) δ 11.86 (s, 1H). 11.67 (s, 1H), 10.46 (s, 1H), 8.46 (d, J=2.1 Hz, 1H), 8.39 (s, 1H), 8.20 (d, J=2.1 Hz, 1H), 8.08 (d, J=8.1 Hz, 1H), 7.94 (d, J=2.6 Hz, 1H), 7.63-7.55 (m, 3H), 7.50 (d, J=7.2 Hz, 1H), 6.90 (d, J=8.9 Hz, 1H), 6.59 (dd, J=3.6, 1.8 Hz, 1H), 4.43 (s, 1H), 4.10 (s, 1H), 2.31 (m. 2H); ¹³C NMR (151 MHz, DMSO) δ 169.68, 160.49, 155.82, 148.55, 143.29, 141.73, 140.81, 138.41, 137.87, 130.03, 128.83, 127.65, 127.15, 126.32, 126.11, 124.85, 122.19, 120.92, 120.08, 117.59, 116.18, 100.82, 40.55, 40.43, 40.30, 40.16, 40.02, 39.88, 39.74, 39.60, 16.09; HRMS (ESI) m/z calcd for C₂₆H₂₁N₄O₃S [M+H]⁺ 469.1334, found 469.1327.

### N-(pyrimidin-5-yl)-4-(1H-pyrrolo[2,3-b]pyridin-5-yl)benzo[b]thiophene-2-carboxamide (CDD-3015)

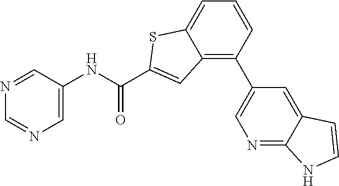 [SMILES: O=C(Nc1cncnc1)c1cc2c(-c3cnc4[nH]ccc4c3)cccc2s1]

Yield 58% (65 mg); white solid; ¹H NMR (600 MHz, DMSO) δ 11.88 (s. 1H), 10.90 (s, 1H). 9.11 (d, J=2.0 Hz, 2H). 8.95 (d, J=2.1 Hz, 1H). 8.47 (s, 2H). 8.20 (t. J=2.1 Hz. 1H), 8.12 (dd, J=8.3, 2.7 Hz, 1H), 7.63 (ddd, J=14.4, 6.8, 2.6 Hz, 2H), 7.53 (dd, J=7.2, 2.4 Hz, 1H), 6.60 (dt, J=3.6, 1.9 Hz, 1H); ¹³C NMR (151 MHz, DMSO) δ 161.37, 154.04, 148.85, 148.58, 143.27, 142.04, 139.31, 138.33, 138.18, 134.50, 128.83, 127.96, 127.72, 127.60, 126.49, 126.27, 122.30, 120.09, 100.83: HRMS (ESI) m/z calcd for C₂₀H₁₄N₅OS [M+H]⁺ 372.0919, found 372.0911.

### N-(3-(dimethylcarbamoyl)-4-hydroxyphenyl)-4-(3-methyl-1H-pyrrolo[2,3-b]pyridin-5-yl)benzo[b]thiophene-2-carboxamide (CDD-3017)

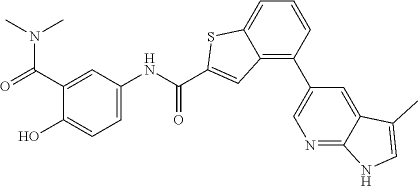 [SMILES: Cc1c[nH]c2ncc(-c3cccc4sc(C(=O)Nc5ccc(O)c(C(=O)N(C)C)c5)cc34)cc12]

Yield 50% (40 mg); white solid: ¹H NMR (600 MHz, DMSO) δ 11.60 (s. 1H), 10.39 (s, 1H), 9.74 (s, 1H), 8.47 (s, 1H), 8.41 (s, 1H), 8.19 (d, J=2.0 Hz, 1H), 8.07 (d, J=8.1 Hz, 1H), 7.59 (t, J=7.7 Hz, 1H), 7.56-7.48 (m, 2H), 7.45 (d, J=2.6 Hz, 1H), 7.39-7.36 (m, 1H), 6.85 (d, J=8.8 Hz, 1H), 2.85 (s, 6H), 2.34 (d, J=1.1 Hz, 3H); ¹³C NMR (151 MHz, DMSO) δ 168.58, 160.27, 150.21, 148.00, 142.44, 141.69, 140.98, 138.50, 137.75, 130.56, 127.96, 127.37, 127.05, 126.33, 124.99, 124.77, 124.74, 123.21, 122.19, 120.98, 120.94, 116.17, 109.64, 49.07, 10.13; HRMS (ESI) m/z calcd for C₂₆H₂₃N₄O₃S [M+H]⁺ 471.1490, found 471.1478.

### N-(3-(dimethylcarbamoyl)-4-hydroxyphenyl)-4-(1H-pyrazolo[3,4-b]pyridin-5-yl)benzo[b]thiophene-2-carboxamide (CDD-3018)

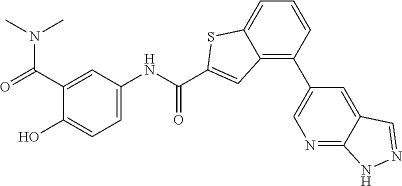 [SMILES: CN(C)C(=O)c1cc(NC(=O)c2cc3c(-c4cnc5[nH]ncc5c4)cccc3s2)ccc1O]

Yield 60% (46 mg); white solid; ¹H NMR (600 MHz, DMSO) δ 13.86 (s. 1H), 10.35 (s, 1H), 9.78 (s, 1H), 8.77 (d, J=2.1 Hz, 1H), 8.49 (d, J=2.1 Hz, 1H), 8.38 (s, 1H), 8.29 (s, 1H), 8.11 (d, J=8.1 Hz, 1H), 7.61 (t, J=7.7 Hz, 1H), 7.53 (dd, J=11.0, 8.1 Hz, 2H), 7.44 (d, J=2.6 Hz, 1H), 6.85 (d, J=8.8 Hz, 1H), 2.90 (d, J=64.6 Hz, 6H); ¹³C NMR (151 MHz, DMSO) δ 168.58, 160.18, 151.76, 150.32, 149.74, 141.77, 141.33, 138.40, 136.57, 134.26, 130.46, 129.14, 127.10, 126.47, 124.81, 124.36, 123.20, 122.73, 121.00, 116.20, 114.88, 38.03, 34.59.; HRMS (ESI) m, z calcd for C₂₄H₂₀N₅O₃S [M+H]⁺ 458.1286, found 458.1278.

### N-(2-(dimethylamino)ethyl)-4-(1H-pyrrolo[2,3-b]pyridin-5-yl)benzo[b]thiophene-2-carboxamide (CDD-3085)

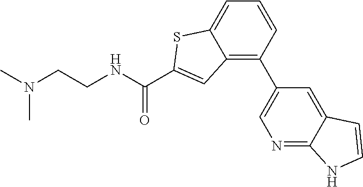 [SMILES: CN(C)CCNC(=O)c1cc2c(-c3cnc4[nH]ccc4c3)cccc2s1]

Yield 54% (53 mg); white solid: ¹H NMR (600 MHz, DMSO) δ 11.85 (s. 1H), 8.79 (t, J=5.7 Hz, 1H), 8.42 (d, J=2.2 Hz, 1H), 8.15 (dd, J=5.4, 1.5 Hz, 2H), 8.03 (d, J=8.1 Hz, 1H), 7.61-7.53 (m, 2H), 7.45 (dd, J=7.3, 1.0 Hz, 1H), 6.58 (dd, J=3.4, 1.8 Hz, 1H), 3.32 (q, J=6.6 Hz, 2H), 2.36 (t, J=6.9 Hz, 2H), 2.15 (s, 6H); ¹³C NMR (151 MHz, DMSO) δ 161.81, 148.50, 143.27, 141.42, 140.96, 138.48, 137.62, 128.75, 128.07, 127.56, 126.77, 126.13, 123.86, 122.14, 120.00, 100.79, 58.66, 45.73, 37.91; HRMS (ESI) m/z calcd for C₂₀H₂₁N₄OS [M+H]⁺ 365.1436, found 365.1442.

### N-(3-((2-(dimethylamino)ethyl)carbamoyl)-4-hydroxyphenyl)-4-(!H-pyrrolo[2,3-b]pyridin-5-yl)benzo[b]thiophene-2-carboxamide (CDD-3086)

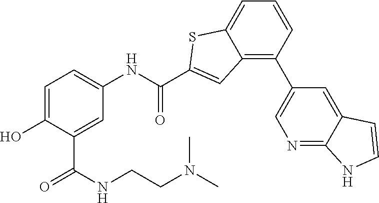 [SMILES: CN(C)CCNC(=O)c1cc(NC(=O)c2cc3c(-c4cnc5[nH]ccc5c4)cccc3s2)ccc1O]

Yield 43% (46 mg); white solid; ¹H NMR (600 MHz, DMSO) δ 11.78 (d, J 16.0 Hz. 2H), 10.45 (s, 1H), 8.80 (t, J=5.8 Hz, 1H), 8.38 (d, J=15.1 Hz, 2H), 8.12 (d, J=2.0 Hz. 1H), 8.07 (d. J=2.6 Hz. 1H), 8.00 (d. J=8.2 Hz. 1H), 7.59-7.50 (m. 3H), 7.43 (d. J=7.3 Hz, 1H), 6.89 (d, J=8.8 Hz, 1H), 6.52 (dd, J=3.4, 1.8 Hz, 1H), 3.60-3.57 (m, 2H), 3.21 (q, J=5.8 Hz, 2H), 2.78 (d, J=4.4 Hz, 6H); ¹³C NMR (151 MHz, DMSO) δ 168.66, 160.55, 155.87, 148.52, 143.28, 141.75, 140.71, 138.50, 137.90, 130.16, 128.84, 128.04, 127.82, 127.66, 127.18, 126.32, 124.94, 122.42, 122.19, 120.08, 117.70, 116.61, 100.82, 56.44, 43.09, 35.00; HRMS (ESI) m/z calcd for C₂₇H₂₆N₅O₃S [M+H]⁺ 365.1436, found 500.17572.

### N-(4-hydroxy-3-(methylcarbamoyl)phenyl)-4-(1H-pyrrolo[2,3-b]pyridin-5-yl)benzo[b]thiophene-2-carboxamide (CDD-3087)

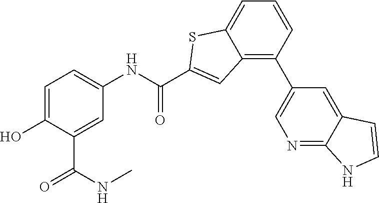 [SMILES: CNC(=O)c1cc(NC(=O)c2cc3c(-c4cnc5[nH]ccc5c4)cccc3s2)ccc1O]

Yield 35% (38 mg); white solid; ¹H NMR (600 MHz, DMSO) δ 12.22 (s. 1H), 11.86 (s, 1H), 10.51 (s, 1H), 8.74 (q, J=4.6 Hz, 1H), 8.48-8.43 (m, 2H), 8.19 (d, J=2.1 Hz, 1H), 8.10-8.05 (m, 2H), 7.63-7.57 (m, 3H), 7.50 (dd, J=7.3, 1.0 Hz, 1H), 6.92 (d, J=8.9 Hz, 1H), 6.59 (dd, J=3.4, 1.8 Hz, 1H), 2.82 (d, J=4.5 Hz, 3H); ¹³C NMR (151 MHz, DMSO) δ 168.88, 160.51, 156.46, 148.55, 143.31, 141.74, 140.77, 138.53, 137.90, 129.84, 128.82, 128.02, 127.82, 127.62, 127.12, 126.28, 124.90, 122.18, 121.95, 120.04, 117.70, 116.19, 100.82, 26.59; HRMS (ESI) m/z calcd for C₂₄H₁₉N₄O₃S [M+H]⁺ 443.1177, found 443.1178.

### N-(4-hydroxy-3-(oxetan-3-ylcarbamoyl)phenyl)-4-(1H-pyrrolo[2,3-b]pyridin-5-yl)benzo[b]thiophene-2-carboxamide (CDD-3112)

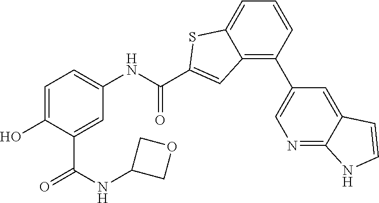 [SMILES: O=C(Nc1ccc(O)c(C(=O)NC2COC2)c1)c1cc2c(-c3cnc4[nH]ccc4c3)cccc2s1]

Yield 15% (10 mg); white solid; ¹H NMR (600 MHz, DMSO) δ 11.80 (s, 1H), 10.44 (s, 1H). 8.39 (s, 1H), 8.35 (d, J=4.5 Hz, 1H), 8.13 (dd, J=6.9, 2.1 Hz, 1H), 8.04 (d. J=5.4 Hz. 1H), 8.02-7.97 (m. 2H), 7.67 (dd, J=8.9, 2.7 Hz. 1H), 7.56-7.49 (m, 2H), 7.43 (ddd. 0.1=7.3, 3.2, 1.0 Hz, 1H), 6.94 (d, J=8.9 Hz, 1H), 6.52 (td, J=3.4, 1.8 Hz, 1H), 4.49 (d, J=8.8 Hz, 1H), 4.45-4.40 (m, 1H), 4.33 (d, J=8.2 Hz, 1H), 3.67-3.57 (m, 1H), 3.51-3.46 (m, 1H); HRMS (ESI) m/z calcd for C₂₆H₂₁N₄O₄S [M+H]⁺ 485.1283, found 485.1283.

### N-(3-((cyclopropylmethyl)carbamoyl)-4-hydroxyphenyl)-4-(1H-pyrrolo[2,3-b]pyridin-5-yl)benzo[b]thiophene-2-carboxamide (CDD-3113)

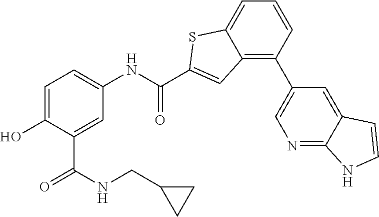 [SMILES: O=C(Nc1ccc(O)c(C(=O)NCC2CC2)c1)c1cc2c(-c3cnc4[nH]ccc4c3)cccc2s1]

Yield 15% (14 mg); white solid, ¹H NMR (600 MHz, DMSO) δ 12.00 (s. 1H), 11.61 (s, 1H), 10.27 (s, 1H), 8.65 (d, J=5.8 Hz, 1H), 8.23-8.16 (m, 2H), 7.95 (d, J=2.1 Hz, 1H), 7.86-7.81 (m, 2H), 7.42-7.31 (m, 3H), 7.25 (d, J=7.2 Hz, 1H), 6.67 (d, J=8.8 Hz, 1H), 6.35 (dd, J=3.5, 1.8 Hz, 1H), 2.93 (t, J=6.2 Hz, 2H), 0.81 (ddd. J=12.3, 8.0, 4.9 Hz, 1H), 0.24-0.17 (m. 2H), 0.01 (t, J=5.2 Hz, 2H); ¹³C NMR (151 MHz, DMSO) δ 168.28, 160.52, 156.57, 148.54, 143.31, 141.74, 140.76, 138.53, 137.89, 129.81, 128.82, 128.02, 127.95, 127.62, 127.13, 126.28, 124.90, 122.19, 122.12, 120.03, 117.71, 116.23, 100.82, 43.91, 11.23, 3.81; HRMS (ESI) m/z calcd for C₂₇H₂₃N₄O₃S [M+H]⁺ 483.1490, found 483.1510.

### N-(3-oxoisoindolin-5-yl)-4-(1H-pyrrolo[2, 3-b]pyridin-5-yl)benzo[b]thiophene-2-carboxamide (CDD-3115)

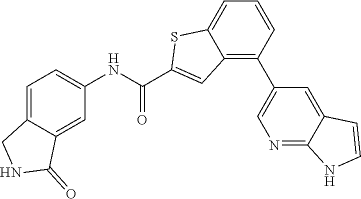 [SMILES: O=C(Nc1ccc2c(c1)C(=O)NC2)c1cc2c(-c3cnc4[nH]ccc4c3)cccc2s1]

Yield 34% (60 mg); white solid: ¹H NMR (600 MHz, DMSO) δ 11.73 (s. 1H), 10.56 (s, 1H), 8.52-8.31 (m, 3H), 8.02 (d, J=73.7 Hz, 3H), 7.76 (d, J=7.9 Hz, 1H), 7.56-7.33 (m, 4H), 6.46 (d, J=5.9 Hz, 1H), 4.22 (s, 2H); ¹³C NMR (151 MHz, DMSO) δ 170.22, 160.87, 148.57, 143.31, 141.87, 140.56, 139.80, 138.68, 138.42, 138.00, 133.61, 128.86, 128.03, 127.64, 127.28, 126.35, 125.36, 124.37, 124.11, 122.20, 120.08, 114.88, 100.84, 45.14.; HRMS (ESI) m/z calcd for C₂₄H₁₇N₄O₂S [M+H]425.1072, found 425.1068.

### 4-(4-(1H-pyrrolo[2,3-b]pyridin-5-yl)benzo[b]thiophene-2-carbonyl)-1-phenylpiperazin-2-one (CDD-3116)

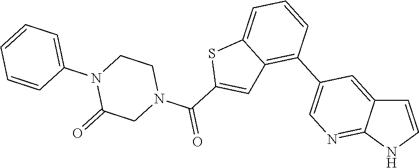 [SMILES: O=C(c1cc2c(-c3cnc4[nH]ccc4c3)cccc2s1)N1CCN(c2ccccc2)C(=O)C1]

Yield 43% (70 mg); white solid; ¹H NMR (600 MHz, CDCl₃) δ 10.54 (s, 1H), 8.52 (s, 1H), 8.14 (d, J=2.0 Hz. 1H), 7.91 (dt, J=8.1, 1.0 Hz, 1H), 7.74 (d, J=0.8 Hz, 1H). 7.56 (dd, J=8.2, 7.2 Hz. 1H), 7.50-7.45 (m, 2H), 7.45-7.39 (m, 2H), 7.32-7.27 (m, 3H), 6.61 (dd, J=3.5, 1.7 Hz, 1H), 4.59 (s, 2H), 4.12 (t, J=5.4 Hz, 2H), 3.84 (t, J=5.4 Hz, 2H); ¹³C NMR (151 MHz, CDCl₃) δ 164.56, 163.87, 148.23, 143.17, 141.33, 141.21, 137.52, 137.26, 135.52, 129.42, 129.23, 129.18, 128.54, 127.45, 126.57, 126.24, 126.01, 125.95, 125.60, 121.49, 120.24, 101.21, 49.27, 30.95; HRMS (ESI) m/z calcd for C₂₆H₂₁N₄O₂S [M+H]⁺ 453.1385, found 453.1402.

### N-(3-((diethylamino)methyl)-4-hydroxyphenyl)-4-(1H-pyrrolo[2,3-b]pyridin-5-yl)benzo[b]thiophene-2-carboxamide (CDD-3117)

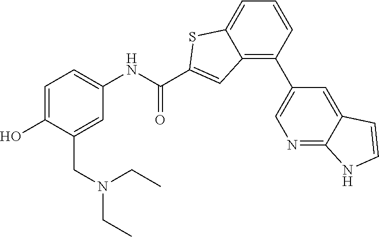 [SMILES: CCN(CC)Cc1cc(NC(=O)c2cc3c(-c4cnc5[nH]ccc5c4)cccc3s2)ccc1O]

Yield 50% (65 mg); white solid; H NMR (600 MHz, DMSO) δ 11.80 (s, 1H), 10.40 (s, 2H), 8.40-8.35 (m, 1H), 8.33 (s, 1H), 8.11 (d, J=2.1 Hz, 1H), 8.00 (d, J=8.1 Hz, 1H), 7.69 (d, J=2.6 Hz, 1H), 7.55-7.49 (m. 2H), 7.44-7.39 (m, 2H), 6.86 (d, J=8.8 Hz, 1H), 6.51 (dd, J=3.4, 1.8 Hz, 1H), 4.12 (s. 2H), 3.01 (q. J=7.1 Hz. 4H), 1.17 (t, J=7.2 Hz, 6H); ¹³C NMR (151 MHz, DMSO) δ 160.43, 158.36, 153.65, 148.54, 143.29, 141.71, 140.87, 138.48, 137.86, 130.59, 128.80, 128.05, 127.66, 127.14, 126.31, 125.67, 124.80, 124.47, 122.18, 120.07, 116.02, 100.80, 51.01, 47.10, 9.15; HRMS (ESI) m/z calcd for C₂₇H₂₇N₄O₂S [M+H]⁻ 471.1854, found 471.1850.

### N-(3-cyano-4-hydroxyphenyl)-4-(1H-pyrrolo[2,3-b]pyridin-5-yl)benzo[b]thiophene-2-carboximidamide (CDD-3126)

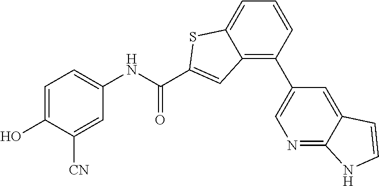 [SMILES: N#Cc1cc(NC(=O)c2cc3c(-c4cnc5[nH]ccc5c4)cccc3s2)ccc1O]

Yield 35% (28 mg): white solid; ¹H NMR (600 MHz, DMSO) δ 11.79 (s, 1H), 10.93 (s, 1H). 10.47 (s, 1H), 8.40-8.37 (m, 1H), 8.32 (s, 1H), 8.12 (d, J=2.0 Hz, 1H), 8.01 (d, J=8.1 Hz. 1H), 7.84 (d. J=2.6 Hz. 1H), 7.65 (dd, J=9.0, 2.7 Hz, 1H), 7.56-7.50 (m, 2H), 7.42 (dd, J=7.3, 1.0 Hz, 1H), 6.94 (d, J=9.0 Hz, 1H), 6.51 (dd, J=3.4, 1.8 Hz, 1H); ¹³C NMR (151 MHz, DMSO) δ 160.61, 157.18, 148.49, 143.22, 141.79, 140.39, 138.41, 137.92, 130.84, 128.88, 128.29, 128.03, 127.67, 127.25, 126.35, 125.14, 125.10, 122.22, 120.11, 117.20, 116.98, 100.84, 98.81; HRMS (ESI) m/z calcd for C₂₃H₁₅N₄O₂S [M+H]⁺ 411.0915, found 411.0922.

### Example 3: Off-DNA Synthesis and Confirmation of JNK Inhibitory Activity

The activity of four compounds from qDOS21 and five compounds from qDOS28_1 were synthesized “off-DNA” and tested in JNK enzyme assays. These nine compounds represent the common structural elements that emerged with good binding inhibition activity from the DEC-Tec selections.

The next step was to confirm that the compounds that bound to JNK1, JNK2 or JNK3 in a competitive manner, relative to bentamapimod, were inhibitors of the ATP-dependent kinase activity in assays. In many cases, the results have been replicated at ThermoFisher using their SelectScreen technology platform. Based on knowledge about JNK inhibitors, and the results of DEC-Tec selections, a target compound profile was developed (Table 2).

TABLE 2
Preliminary target compound profile (TCP;
IC₅₀ nM) for next generation JNK inhibitor, based
on K_{d} select results with BEND and TANZ

| | JNK1 | JNK2 | JNK3 | |
|---|---|---|---|---|
| Kinase | (MAPK8) | (MAPK9) | (MAPK10) | Other MAPK |
| goal IC₅₀ | <10 | >10 | <10 | >1,000 |
| BEND* | 270 | 170 | 130 | >1,000 |
| TANZ* | 5.3 | 0.52 | 0.07 | >1,000 |

### Example 4: Kinase and Binding Assays

JNK1, JNK2, and JNK3 hydrolyze ATP to ADP without any peptide substrates. This allows a very robust and fast endpoint assay where the remaining concentration of ATP is measured (BMG ClarioStar Plus plate reader) using the Kinase-Glo Assay kit from Promega (V6711). A luminescence signal is measured, which is produced by the catalytic conversion of beetle luciferin to oxyluciferin by luciferase. Potential inhibition of luciferase (i.e., false negatives) is easily detected, because assays without kinase are always included, and less ATP than is present in this control reaction could also indicate an inhibition of luciferase, which can be excluded by a counter screen with only luciferase. A peptide substrate-independent luminescence assay to measure intrinsic ATPase activity has been previously confirmed as a reliable indicator of catalytic kinase activity.

To mitigate violation of the linear initial velocity principle, which is a problem in end-point assays when doing dose-responses, the enzyme concentration was carefully optimized, in a total assay volume of 25 μL. The assay was performed using 10 μM ATP, 0.02% Tween-20, kinase buffer from Cell Signaling (#9802) in white 384-well plates. The kinase reaction was done for 30 or 60 min at 30° C., and detection of leftover ATP was measured according to Promega's protocol. ADP concentrations were calculated from controls that did not contain any kinase. Fractional activities were obtained from normalized data. Non-linear regression (GraphPad Prism7) and a modified Morrison equation for tight binding was used to determine apparent Kᵢ values for inhibitors, assuming no particular mode of inhibition. An orthogonal binding assay (LanthaScreen) was outsourced to ThermoFisher Scientific's SelectScreen Services to determine IC₅₀ values.

Results from the compounds that were identified as enriched and had been synthesized for validation of qDOS18_2 results are provided herein (Tables 3a-3b). None of the lower priority compounds from this library had activity comparable to bentamapimod (CDD-939) or tanzisertib (CDD-985).

TABLE 3a
K_{d} (nM) values for JNK1, JNK2, JNK3 of compounds
identified in the qDOS21 and qDOS28 libraries

| | | | | Solubility |
|---|---|---|---|---|
| Compound | JNK1 | JNK2 | JNK3 | (μM) |
| CDD-2288 | 0.36 ± 0.11 | 1.63 ± 0.14 | 1.41 ± 0.07 | 1.0 |
| CDD-2325 | 0.28 ± 0.12 | 1.27 ± 0.14 | 1.18 ± 0.09 | 2.3 |
| CDD-2343 | NA | NA | NA | — |
| CDD-2344 | NA | NA | NA | — |
| CDD-2346 | 0.23 ± 0.08 | 0.94 ± 0.22 | 0.65 ± 0.16 | 166.8 |
| CDD-2347 | 0.39 ± 0.11 | 1.7 ± 0.12 | 1.38 ± 0.21 | 134.2 |
| CDD-2387 | 2.96 ± 0.32 | 28.8 ± 1.8 | 8.44 ± 0.66 | 253.9 |
| CDD-2388 | NA | NA | NA | — |
| CDD-2426 | NA | NA | NA | — |
| CDD-2461 | 0.2 ± 0.18 | 0.32 ± 0.12 | 0.33 ± 0.03 | 1.8 |
| CDD-2482 | 0.25 ± 0.04 | 0.74 ± 0.09 | 0.45 ± 0.04 | 176 |
| CDD-2484 | NA | NA | NA | — |
| CDD-2485 | 0.12 ± 0.09 | 0.95 ± 0.16 | 0.73 ± 0.02 | — |
| CDD-2486 | 1.7 ± 0.2 | 0.67 ± 0.09 | 0.86 ± 0.05 | 0.3 |
| CDD-2487 | NA | NA | NA | — |
| CDD-2488 | 3.1 ± 0.8 | 3.08 ± 0.16 | 4.34 ± 0.6 | 124.2 |
| CDD-2489 | NA | NA | NA | — |
| CDD-2490 | 1.52 ± 0.3 | 5.37 ± 0.42 | 3.53 0.37 | 0.4 |
| CDD-2491 | 0.28 ± 03 | 0.43 ± 0.09 | 1.41 ± 0.12 | 2.5 |
| CDD-2519 | NA | NA | NA | — |
| CDD-2520 | NA | NA | NA | — |
| CDD-2574 | 1.69 ± 0.39 | 12.55 ± 0.68 | 6.56 ± 0.85 | 11.1 |
| CDD-2575 | 1.15 ± 0.28 | 1.76 ± 0.15 | 2.76 ± 0.51 | 40.6 |
| CDD-2627 | 3.74 ± 0.93 | 14.8 ± 0.95 | 9.0 ± 2.4 | 40.3 |
| CDD-2628 | 1.83 ± 0.26 | 1.6 ± 0.17 | 0.52 ± 0.05 | 7.4 |
| CDD-2629 | 7.8 ± 1.4 | 15.4 ± 1.4 | 9.9 ± 1.3 | 0 |
| CDD-2630 | 0.68 ± 0.08 | 2.47 ± 0.25 | 1.63 ± 0.26 | 1.6 |
| CDD-2631 | 5.9 ± 1.1 | 27.8 ± 3.6 | 12.7 ± 1.2 | — |
| CDD-2632 | 4.91 ± 0.9 | 3.5 ± 1.4 | 5.82 ± 0.92 | 0.6 |
| CDD-2634 | 0.29 ± 0.13 | 1.81 ± 0.19 | 2.62 ± 0.28 | 1.1 |
| CDD-2635 | 0.16 ± 0.05 | 1.54 ± 0.13 | 0.76 ± 0.22 | 2.6 |
| CDD-2636 | 0.57 ± 0.1 | 1.93 ± 0.14 | 0.54 ± 0.09 | 4.9 |
| CDD-2728 | 0.24 ± 0.05 | 3.79 ± 0.29 | 0.95 ± 0.08 | 19.4 |
| CDD-2729 | 1.2 ± 0.16 | 6.5 ± 0.63 | 1.93 ± 0.14 | 0 |
| CDD-2730 | 0.58 ± 0.08 | 5.33 ± 0.52 | 1.86 ± 0.18 | 0.3 |
| CDD-2731 | 0.17 ± 0.05 | 1.28 ± 0.15 | 0.81 ± 0.05 | 3.2 |
| CDD-2732 | 14.3 ± 1.6 | 67.0 ± 11 | 24.3 ± 1.9 | 0 |
| CDD-2855 | 1.52 ± 0.15 | 12.2 ± 1.3 | 2.68 ± 0.20 | 0 |
| CDD-2856 | 0.27 ± 0.08 | 3.14 ± 0.33 | 1.03 ± 0.07 | 3.8 |
| CDD-2857 | 11.3 ± 3.4 | 49.8 ± 9.7 | 15.7 ± 1.2 | — |
| CDD-2935 | 27.0 ± 1.6 | 128 ± 15 | 32.2 ± 1.0 | 8.8 |
| CDD-2936 | 1.62 ± 0.31 | 9.09 ± 0.53 | 3.2 + 0.3 | 0 |
| CDD-2937 | 0.28 ± 0.05 | 4.53 ± 0.55 | 1.67 ± 0.06 | 0 |
| CDD-2938 | 4.6 ± 1.9 | 1.22 ± 0.27 | 1.41 ± 0.30 | 5.4 |
| CDD-3012 | 0.52 ± 0.12 | 4.84 ± 0.49 | 2.08 ± 0.18 | 2.0 |
| CDD-3013 | 0.21 ± 0.03 | 0.94 ± 0.09 | 0.75 ± .07 | 0.4 |
| CDD-3014 | 0.58 ± 0.08 | 5.74 ± 0.5 | 2.92 ± 0.21 | 0.4 |
| CDD-3015 | 2.34 ± 0.3 | 6.84 ± 0.81 | 3.18 ± 0.14 | 0.3 |
| CDD-3016 | 0.65 ± 0.15 | 5.77 ± 0.8 | 1.48 ± 0.14 | 9.7 |
| CDD-3017 | 0.39 ± 0.12 | 2.23 ± 0.12 | 2.7 ± 0.29 | 0 |
| CDD-3018 | 3.58 ± 0.47 | 11.4 ± 1 | 5.17 ± 0.33 | 1.5 |
| CDD-3085 | 13.1 ± 1.2 | 42.8 ± 8.3 | 30.3 ± 2.7 | 170 |
| CDD-3086 | 0.67 ± 0.14 | 5.98 ± 0.42 | 5.21 ± 0.46 | 2.0 |
| CDD-3087 | 1.96 ± 0.53 | 11.38 ± 0.81 | 4.54 ± 0.5 | 0.4 |
| CDD-3111 | 0.07 ± 0.05 | 0.73 ± 0.1 | 0.32 ± 0.04 | 3.8 |
| CDD-3112 | 2.84 ± 0.49 | 13.5 ± 0.8 | 6.4 ± 0.6 | 1.6 |
| CDD-3113 | 10.4 ± 1.4 | 40.7 ± 5.5 | 10.9 ± 0.7 | 0 |
| CDD-3114 | 4.0 ± 0.8 | 20.6 ± 3.1 | 9.0 ± 0.7 | 0 |
| CDD-3115 | 1.5 ± 0.2 | 6.3 ± 0.6 | 4.6 ± 0.7 | 1.4 |
| CDD-3116 | 54.5 ± 5.4 | 447 ± 65 | 114 ± 7 | 4.2 |
| CDD-3117 | 0.9 ± 0.2 | 2.1 ± 0.3 | 2.9 ± 0.4 | 3.4 |

TABLE 3b
Ki(app) values for JNK inhibitors (CDD-939 and CDD-985) and
compounds identified in the qDOS11 and qDOS21 libraries

| Compound | JNK1 (μM) | JNK2 (μM) | JNK3 (μM) | Solubility (μM) |
|---|---|---|---|---|
| CDD-939 | 0.22 | 0.64 | 0.039 | — |
| CDD-985 | 0.03 | 0.01 | 0.4 | — |
| CDD-2272 | 0.86 | — | — | — |
| CDD-2483 | 0.194 | 0.339 | 0.167 | 0.6 |
| CDD-3126 | 0.03 | — | — | >200 |

### Example 5: Optimization of JNK Inhibitors

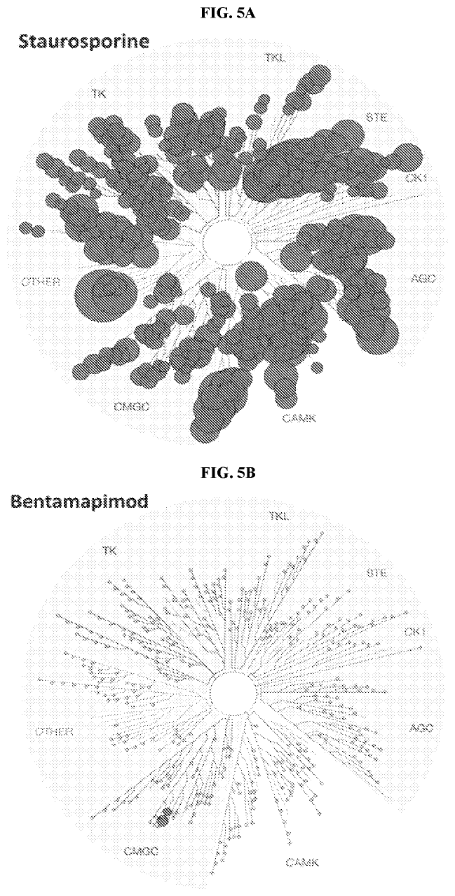

JNK inhibitors have been optimized to achieve high affinity for the JNK enzyme, and in parallel to meet the criteria for isoform selectivity, while demonstrating high selectivity among unrelated kinases. The efficacy and selectivity of BEND and TANZ were used to define a realistic preliminary target compound profile (Table 2) for the next generation of JNK1-3 inhibitors for the treatment of endometriosis. BEND and TANZ were evaluated using Eurofins KINOMEscan™ and KdSelect™ assays (FIG. 5 and Table 2) The presentation of results in the Treespot dendrogram (Eurofins) provides a comprehensive visual interpretation of the selectivity of BEND and TANZ relative to staurosporine (non-selective).

The results from successive Eurofins evaluations were used to refine the medicinal chemistry goals and strategies for synthesis of next generation JNK inhibitors. The KINOMEscan results show that BEND is highly selective among 480 kinases tested, but less potent on JNK1-3 than desired (Table 2: FIG. 5). The JNK-2.3 inhibition constants for TANZ are adequate (Table 2; FIG. 5), but the K_{d} of TANZ for JNK1 is suboptimal. The imbalanced inhibition of JNK1/JNK2 has been proposed as a contributing cause for termination of TANZ clinical development.

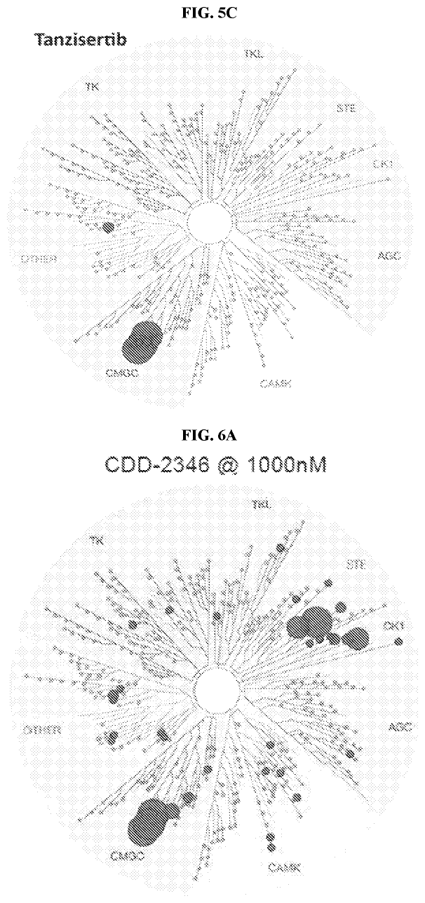

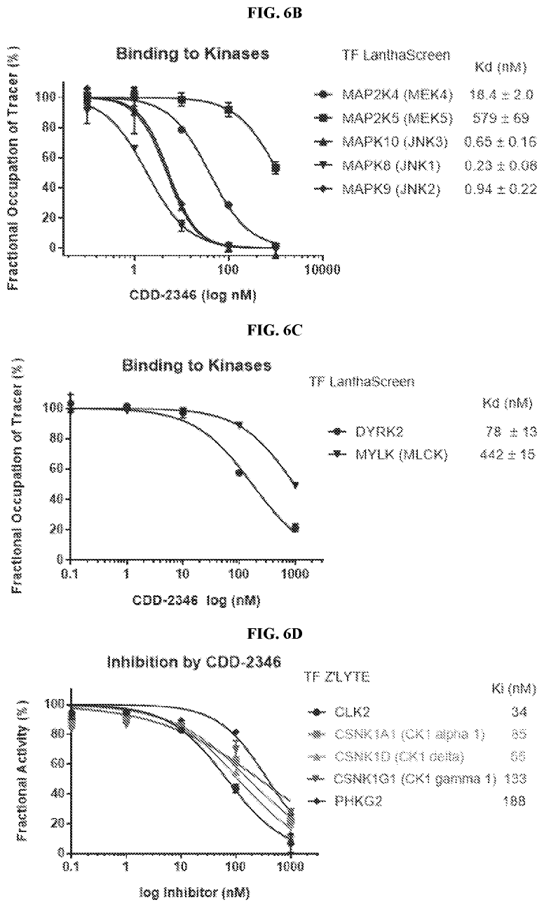

One of the earliest compounds to be synthesized with favorable enzymatic activity towards JNK1 and JNK3 was CDD-2346. A KINOMEscan was performed for CDD-2346 to establish whether the selectivity of this chemical scaffold was suitable or required optimization. Results from this KINOMEscan suggested that the selectivity of CDD-2346 was inadequate and required optimization (FIGS. 6A-6D). To confirm that the activity against each kinase was a true result and not a false positive, CDD-2346 was evaluated in kinase assays using ThermoFisher SelectScreen platform. The results from these assays are provided herein (Table 4). The important innovative steps described are the optimization of selectivity a) for JNK isoforms; b) for kinases considered “in-pathway”; and c) to avoid kinases considered “out-of-pathway”.

TABLE 4
Kinase selectivity K_{d} (nM) profile for exemplary JNK inhibitors

| | CDD- | CDD- | CDD- | CDD- | CDD- | CDD- |
|---|---|---|---|---|---|---|
| Kinase | 2346 | 2347 | 2387 | 2482 | 2486 | 2491 |
| CLK2 | 34 | ND | ND | 17 | 130 | 1175 |
| CSNK1A1 | 85 | ND | ND | 70.5 | 41250 | ND |
| CSNK1D | 55 | ND | ND | 30 | 2350 | 8350 |
| CSNKIE | 48 | 3181 | 2662 | 29 | 235 | ND |
| CSNK1G1 | 133 | ND | ND | 52 | 14400 | 95000 |
| DYRK2 | 78 | ND | ND | 48 | 288 | ND |
| MEK4 | 18 | 483 | 262 | 9.0 | 26.2 | 60.2 |
| MEK5 | 579 | ND | 40700 | 547 | 4040 | ND |
| MYLK | 442 | ND | ND | 501 | 942 | ND |
| PHKG2 | 188 | ND | ND | 110 | 1950 | ND |
| hVPS34 | 109 | 776 | 130 | 255 | 1135 | 38050 |
| PIP5K1C | 811 | ND | ND | 13200 | 1150 | ND |
| JNK2 | 0.23 | 0.39 | 2.9 | 0.25 | 3.1 | 0.28 |
| JNK1 | 0.94 | 1.7 | 28 | 0.74 | 3.08 | 0.43 |
| JNK3 | 0.65 | 1.38 | 8.4 | 0.45 | 4.34 | 1.41 |

The activity of hit compound CDD-2346 failed to meet the selectivity criteria. The importance of disclosing the lack of selectivity for CDD-2346 for JNK1-JNK3 isoforms as well as for other kinases is to demonstrate the intentional process applied in this discovery effort to develop the selectivity among JNK inhibitors. Other discovery groups have pursued a strategy to identify “pan-specific” JNK inhibitors or compounds that inhibit only JNK3. Without wishing to be limited by any theory, both of these strategies would fail to meet the target product profile to influence the peripheral inflammatory component of the disease and the central nervous system component of pain perception.

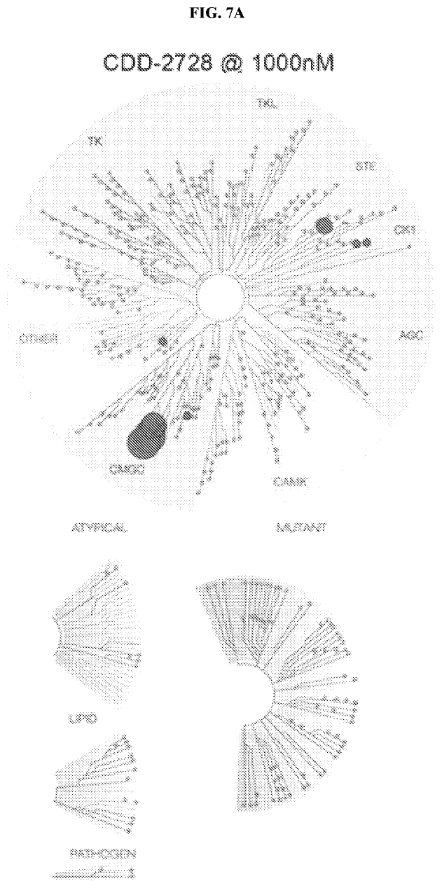

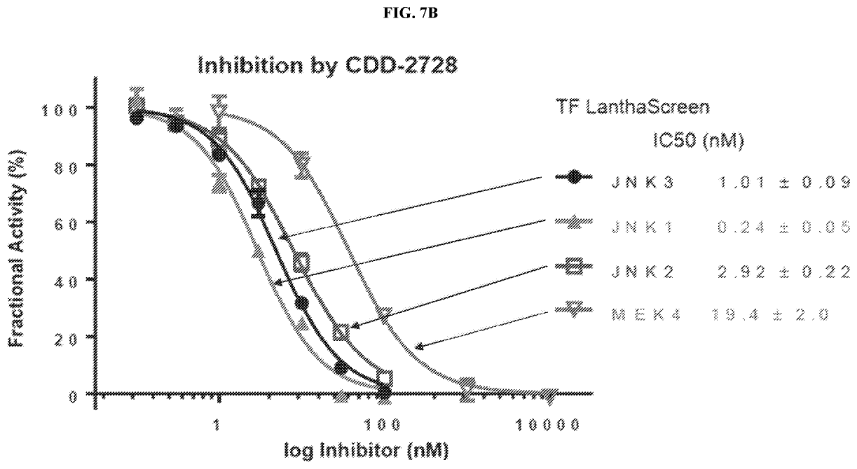

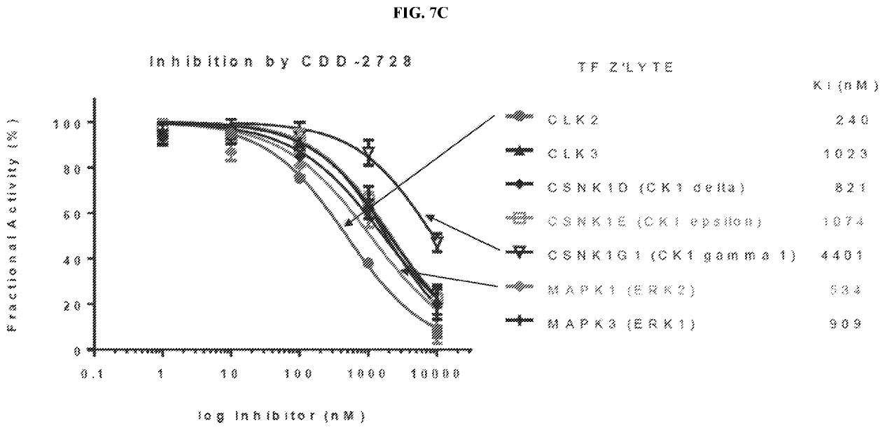

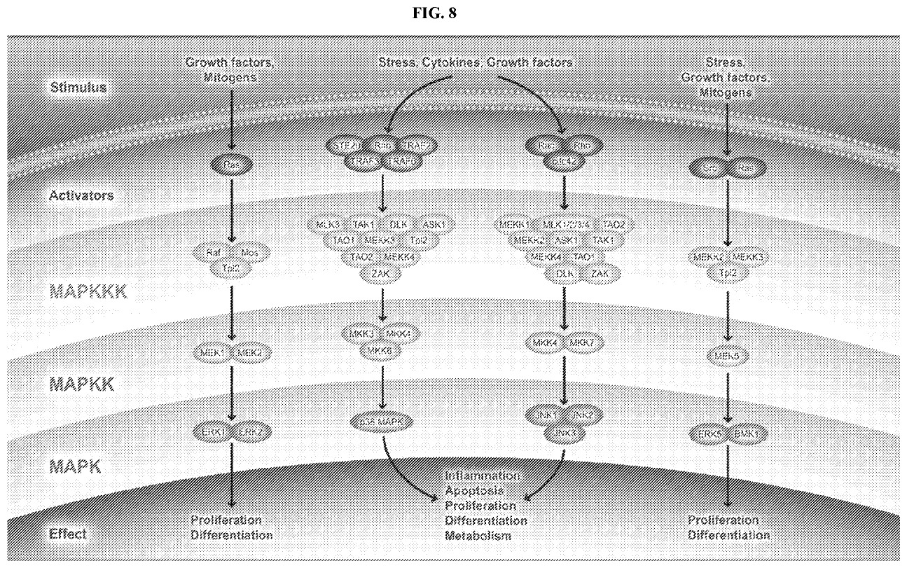

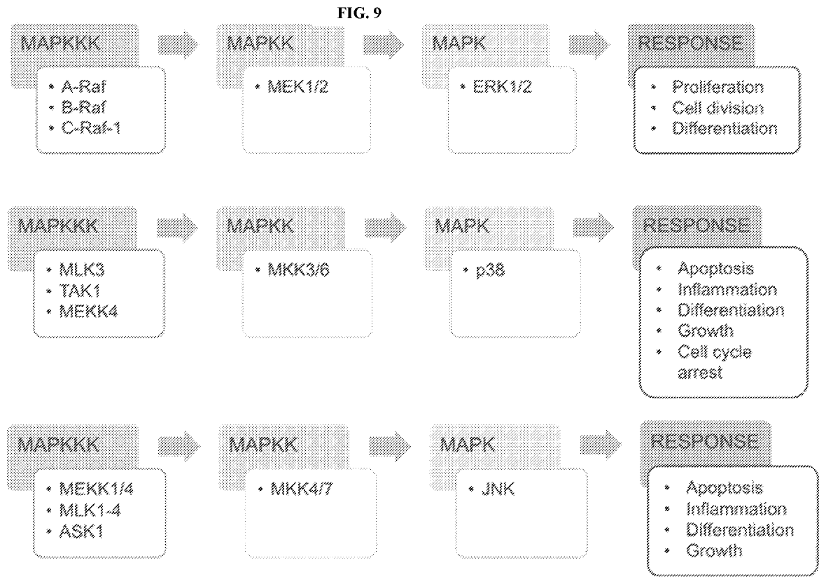

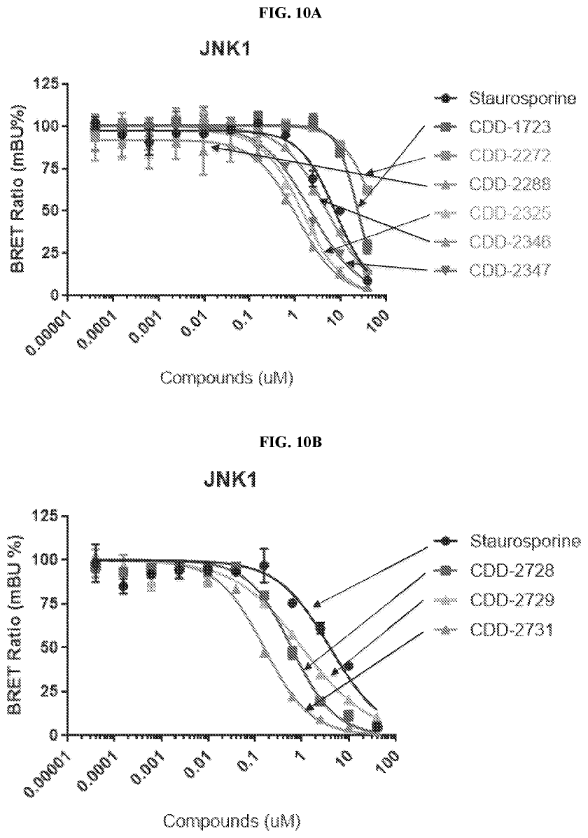

As can be seen as part of the hit optimization process described herein, CDD-2491 emerged with much improved selectivity against unrelated kinases, but failed to establish selectivity desired for JNK1, JNK2 and JNK3. Continued optimization of the JNK inhibitors resulted in CDD-2728, an inhibitor that satisfied potency and selectivity criteria (FIGS. 7A-7C).

The results demonstrate the improved selectivity of CDD-2728 achieved through optimization of JNK inhibitors as demonstrated in the KINOMEscan platform (Eurofins) which identified significant improvements in selectivity toward the JNK subgroup, compared with CDD-2346. No activity of CDD-2728 was detected for Atypical, Mutant, or Lipid kinases. To confirm CDD-2728 inhibition results in the KINOMEscan analysis, individual binding assays were performed on the kinases to which CDD-2728 bound using ThermoFisher SelectScreen. Results from this confirmation assay demonstrated that the selectivity for “in-pathway” target was 7-fold, and the selectivity for five of the “out-of-pathway” kinase targets was over 500-fold (Table 5).

TABLE 5
Inhibition (nM, K_{d}) of JNK1, JNK2, JNK3, ERK1/2, MEK4,
CLK2/3, CSNK1D, CSNK1E, and CSNK1G1 by CDD-2728

| | JNK1 | JNK2 | JNK3 | ERK1 | ERK2 | MEK4 | CLK2 | CLK3 | CSNK1D | CSNK1E | CSNK1G1 |
|---|---|---|---|---|---|---|---|---|---|---|---|
| K_{d} (nM) | 0.31 | 3.79 | 0.97 | 909 | 534 | 18 | 240 | 1023 | 821 | 1074 | 4401 |

TABLE 6
Selectivity ratio (SR) of CDD-2728 for “in-pathway”
and “out-of-pathway” relative to JNK1 inhibition
(Kd for JNK1 = 1.0 nM)

| Enzyme^{a} | SR | Enzyme^{b} | SR | Opportunistic | SR |
|---|---|---|---|---|---|
| CLK3 | 568 | MEK4 | 7.6 | CLK2 | 133 |
| CSNK1D | 511 | JNK1 | 1.0 | ERK1 | 305 |
| CSNK1E | 596 | JNK3 | 2.0 | ERK2 | 505 |
| RIPK4 | >500 | JNK2 | 3.0 | | |

^{a}out-of-pathway;
^{b}in-pathway.

TABLE 7
NanoBRET cellular data of JNK1 IC₅₀ (μM)

| Compound | JNK1 IC₅₀ (μM) |
|---|---|
| Staurosporine | 4.0 |
| CDD-1723 | 24 |
| CDD-2272 | 60 |
| CDD-2288 | 0.83 |
| CDD-2325 | 1.4 |
| CDD-2346 | 5.1 |
| CDD-2347 | 2.2 |
| CDD-2728 | 0.599 |
| CDD-2729 | 1.0 |
| CDD-2731 | 0.156 |

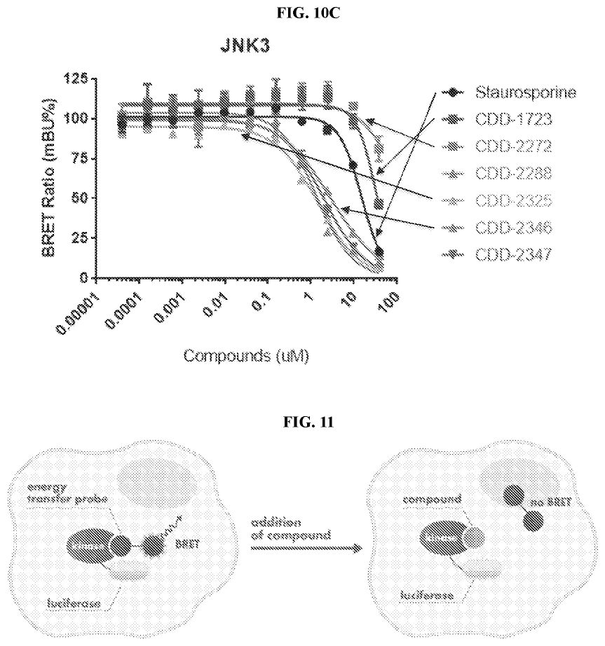

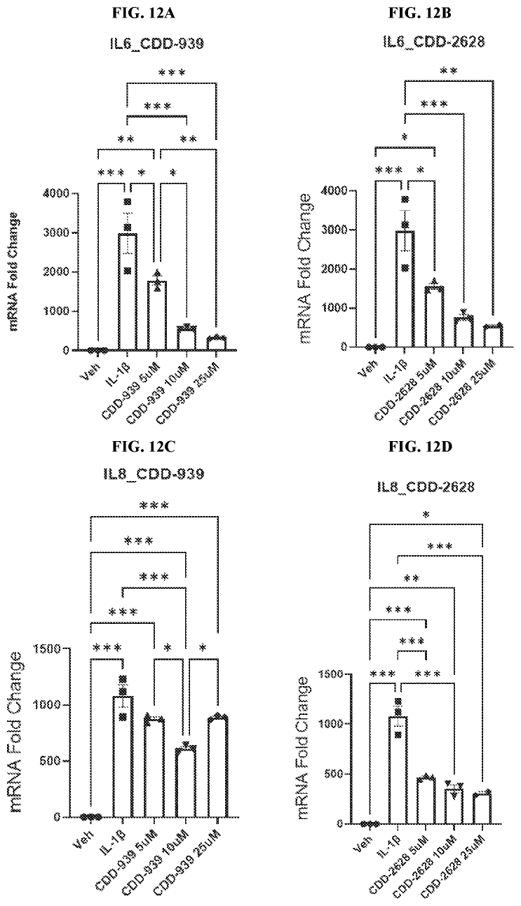

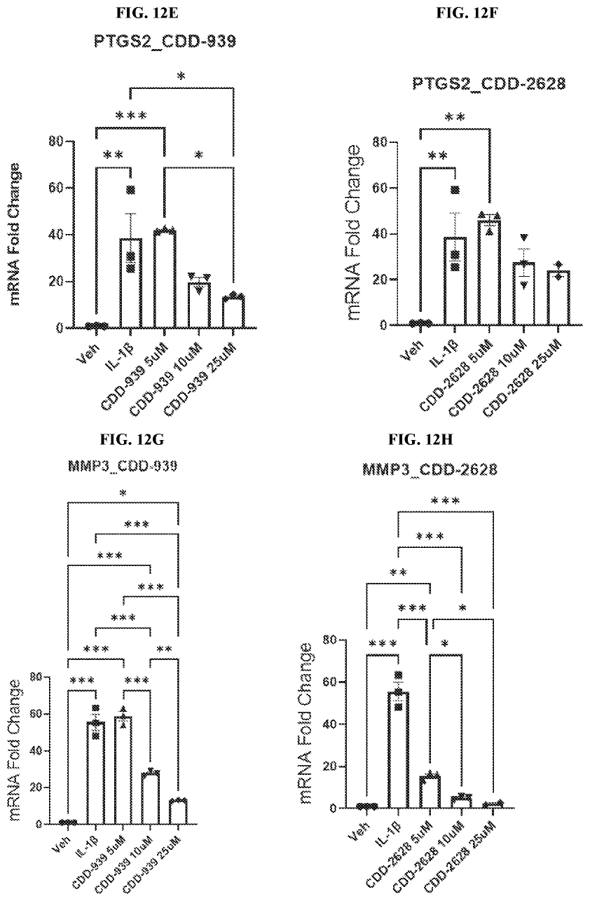

The definition of “in-pathway” was based on published signaling pathways for JNK shown in FIG. 11 and FIGS. 12A-12J. This diagram is consistent with current understanding of MEK4 (i.e., MKK4) activity as the dominant upstream kinase that phosphorylates JNK isoforms, while MEK1 (i.e., MKK1) is the dominant upstream kinase that phosphorylates ERK1/2 activity. ERK1/2 activity in this case is considered “opportunistic” because of the demonstrated association of the MEK/ERK pathway in endometriosis and the grouping of ERK1/2 in the same family of MAPK as JNK. These results position CDD-2728 as a first of its kind of inhibitor that inhibits the priority target pathway (i.e., JNK1-3 and MEK4) with only modest inhibition of ERK1/2, also known to be relevant in models of inflammation and invasion.

This selectivity profile distinguishes CDD-2728 from multiple other MEK inhibitors that lack selectivity among the multiple MEK isoforms. Although the drawings included herein indicate that MEK4 is an upstream kinase for phosphorylation of NF-kappaB pathway, there is little evidence to support this or kinases connected with growth or proliferation (e.g.-mTOR, Akt, and PI3K). CDD-2728 represents a selective JNK-inhibitor and MEK4 inhibitor that is far more selective than pazopanib among the MEK family of kinases, and uniquely selective among JNK1 and JNK3 with less activity on JNK2, as defined for the present target compound profile.

Among the 192 assays, only JNK1, JNK2, and JNK3 were inhibited by CDD-3013 at a concentration of 1 μM (MEK4 is not one of the 192 kinases evaluated in the NanoBRET assay) (Table 8).

TABLE 8
Kinase inhibition profile of CDD-3013 at 1 μM

| Kinase | Average IC₅₀ (μM) |
|---|---|
| MAPK8 | 95.73 |
| MAPK9 | 94.12 |
| MAPK10 | 73.94 |
| S1 | 24.75 |
| T1 | 22.94 |
| M1 | 22.72 |
| C1 | 19.76 |
| C2 | 19.16 |
| M2 | 14.17 |
| M3 | 13.99 |
| N1 | 13.79 |
| G1 | 11.08 |
| C1 | 10.85 |
| P1 | 9.97 |
| S1 | 9.25 |
| J1 | 9.24 |

### Example 6: Demonstrating the Therapeutic Opportunity of CDD-JNK Inhibitors in Cellular Assays

The unique selectivity profile of the compounds of the present disclosure allows the compounds (e.g., CDD-2728) to effectively interrupt the MEK4/JNK pathway, and avoid overlapping selectivity onto MEK1/2, MEK3/6, and MEK5 pathways, and their downstream targets. Compounds of this profile have not been available before CDD-2728 that allow better therapeutic control of the MEK4/JNK pathway, associated with apoptosis, inflammation, and disrupted differentiation and growth of cells comprising endometriotic lesions.

The results provided herein demonstrate that several JNK inhibitors synthesized to date are cell permeable and are potent on endpoints associated with endometriosis. Among JNK inhibitors that were synthesized early in the drug discovery campaign, CDD-2346 was superior to staurosporine for inhibition of binding to JNK1, JNK2, and JNK3 in the Promega NanoBRET™ Target Engagement Intracellular Kinase Assay (Table 9).

TABLE 9
IC₅₀ values determined for JNK inhibitors by displacement
of tracer K10 in Promega NanoBRET assay

| | JNK1 IC₅₀ | JNK2 IC₅₀ | JNK3 IC₅₀ |
|---|---|---|---|
| Compound | (μM) | (μM) | (μM) |
| CDD-939 | ND | ND | ND |
| (bentamapimod) | | | |
| CDD-985 | 3.3 | 0.193 | 0.351 |
| (tanzisertib) | | | |
| staurosporine | 7.4 | 10 | 16 |
| CDD-1723 | 24 | 32 | 38 |
| CDD-2272 | 60 | 58 | ND |
| CDD-2288 | 0.83 | 0.9 | 1.5 |
| CDD-2325 | 1.4 | 0.89 | 1.2 |
| CDD-2346 | 5.1 | 1.7 | 3 |
| CDD-2387 | ND | ND | ND |
| CDD-2347 | 2.2 | 0.99 | 2 |
| CDD-2461 | 49.5 | 5.3 | ND |
| CDD-2486 | 0.77 | 0.442 | 0.468 |
| CDD-2488 | 66 | 13.5 | 34.8 |
| CDD-2490 | 6.3 | 2 | 29.3 |
| CDD-2574 | 7.3 | 5.4 | 5.9 |
| CDD-2575 | 2.2 | 2.3 | 4.8 |
| CDD-2627 | 14.8 | 6.1 | 23 |
| CDD-2628 | 0.5 | 0.52 | 0.566 |
| CDD-2630 | 0.43 | 0.23 | 0.149 |
| CDD-2634 | 0.172 | 0.161 | 0.119 |
| CDD-2635 | 0.186 | 0.325 | 0.160 |
| CDD-2636 | 1.2 | 0.82 | 1.2 |
| CDD-2728 | 0.599 | 0.57 | 0.534 |
| CDD-2729 | 1 | 1 | 0.781 |
| CDD-2731 | 0.156 | 0.156 | 0.124 |
| CDD-2855 | 4.909 | 4.2 | 5.3 |
| CDD-2856 | 1 | 1.1 | 2.2 |
| CDD-2936 | 0.702 | 0.7797 | 1.139 |
| CDD-2937 | 1.6 | 1.459 | 1.126 |
| CDD-2938 | 11.5 | 16.07 | 15.39 |
| CDD-3012 | 3.8 | 3.6 | 6 |
| CDD- 3013 | 0.133 | 0.169 | 0.140 |
| CDD-3014 | 0.789 | 0.738 | 0.960 |
| CDD-3015 | 3.5 | 3.2 | 8.2 |
| CDD-3016 | ND | ND | ND |
| CDD-3017 | 0.254 | 0.228 | 0.235 |
| CDD-3018 | ND | 10.9 | 31.7 |

In this assay format, bentamapimod interferes with the assay readout of the tracer through the cyano group of bentamapimod, and staurosporine is used as a reference control. The IC₅₀ values for various compounds described herein are summarized in Table 9. Optimization of JNK inhibitors improved the NanoBRET IC₅₀ for JNK1 by 8.5-fold (CDD-2346/CDD-2728). Although CDD-2731 and CDD-3013 exhibit better cell-permeable binding to JNK1 in the NanoBRET assay, this compound is rapidly metabolized in liver microsomes.

These studies indicate that NanoBRET assays are an innovative technical platform to measure target engagement of CDD JNK inhibitors with the target JNK1, JNK2 or JNK3. As further evidence that JNK inhibitors address disease-relevant endpoints, biological responses of JNK inhibitors are provided herein, as measured in target tissue cellular models. 12Z immortalized endometriotic epithelial cells and patient-derived endometriotic stromal cells have been cultured with reference compound bentamapimod, CDD-2628, and CDD-2728. To exemplify the effects of CDD-2628 and CDD-2728 in the endometrial cell cultures, gene expression was measured using qPCR (expressed as fold-change relative to GAPDH, a housekeeping gene that is not affected by culture treatment effects) that reflect synthesis of inflammatory lipids (PTGS2), transformation of cells to a more migration and invasive potential (MMP-3) and chemoattractant cytokines to recruit pro-inflammatory immune cells (IL-6, IL-8).

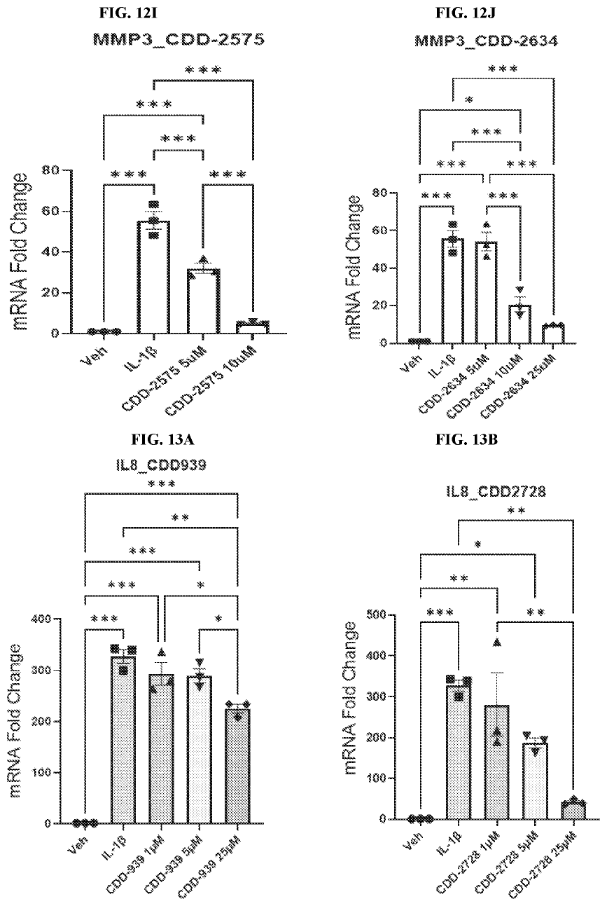

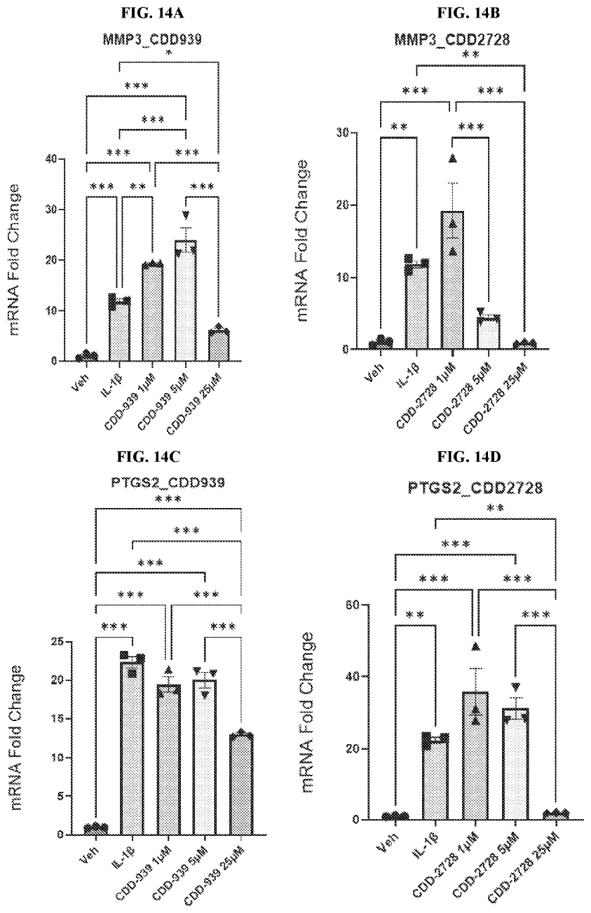

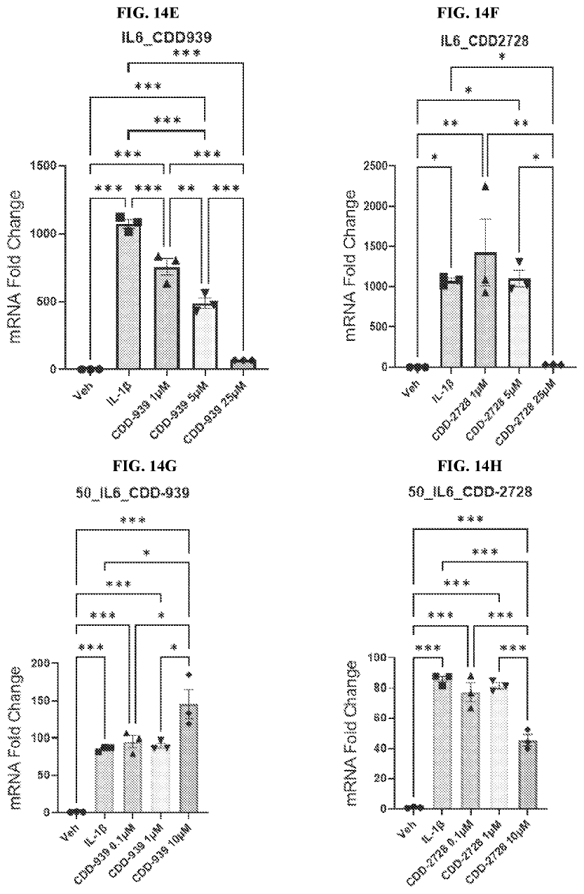

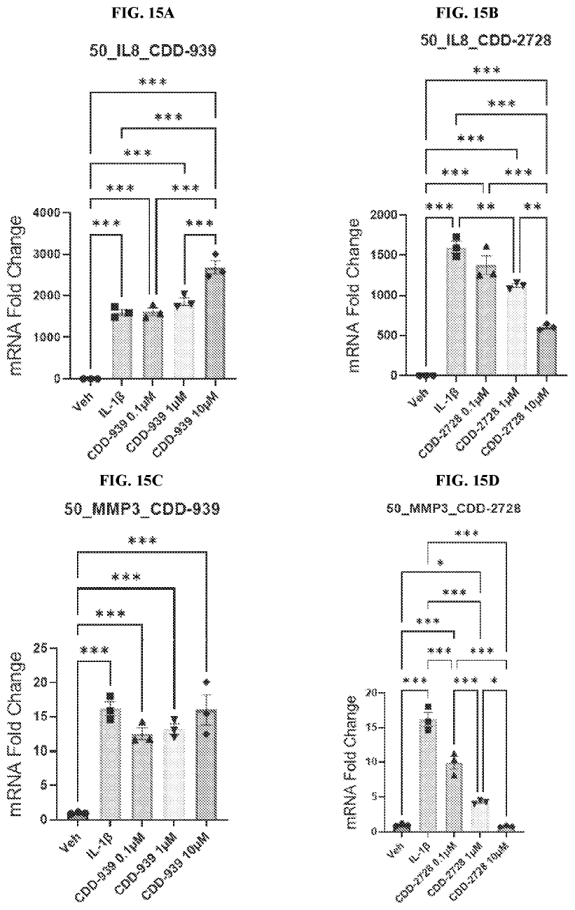

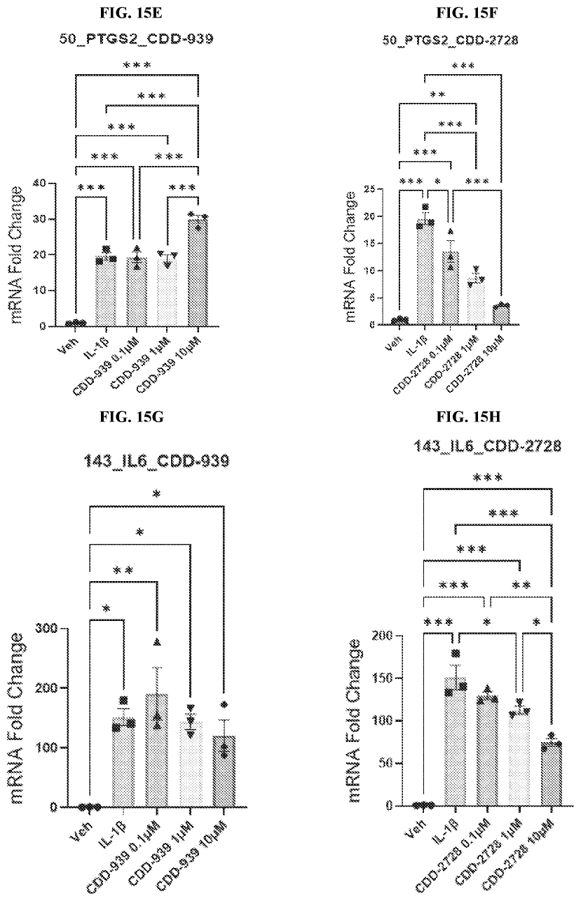

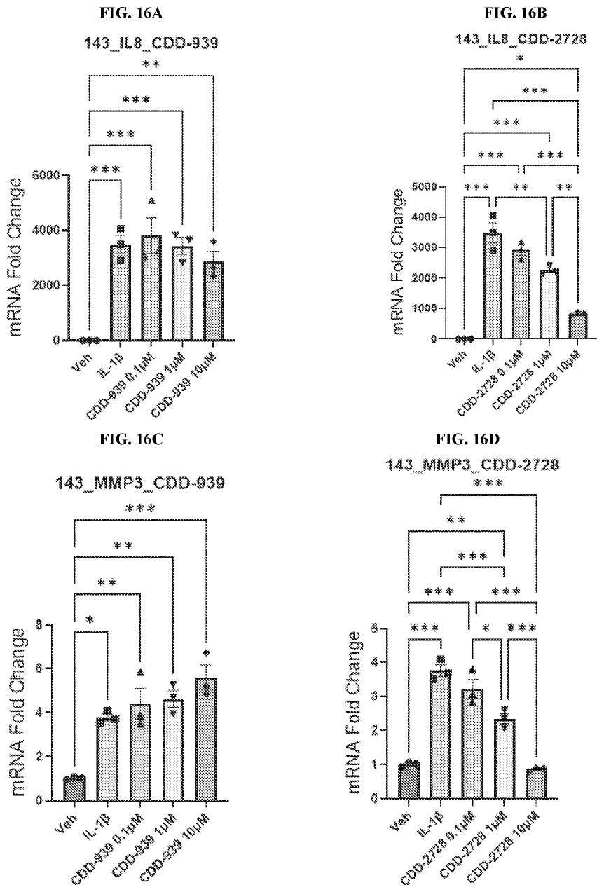

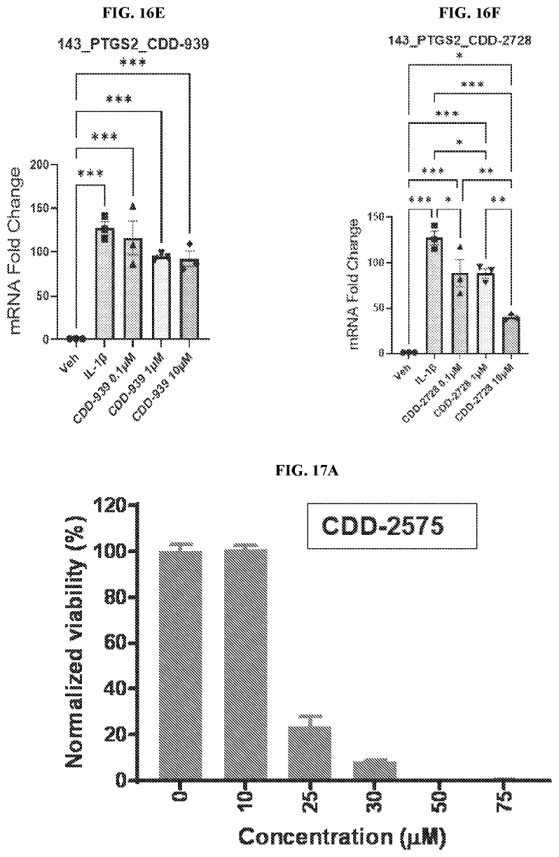

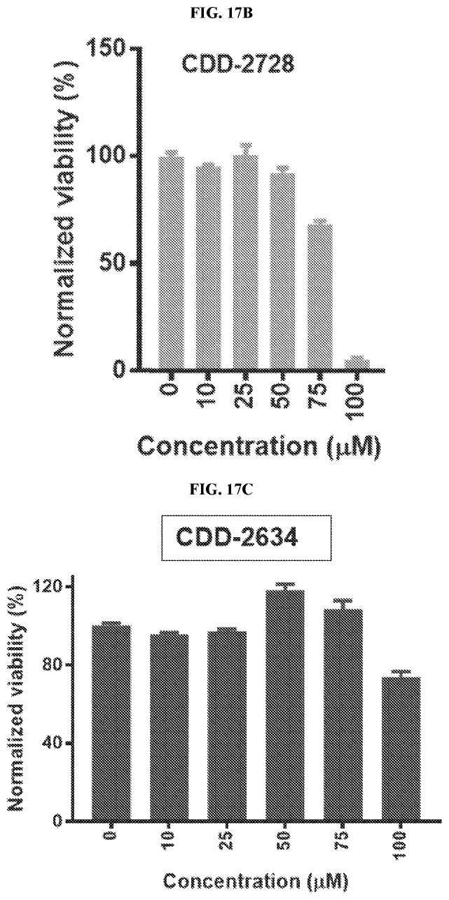

In the culture systems employed in FIGS. 12A-12J, FIGS. 13A-13B, FIGS. 14A-14H, FIGS. 15A-15H, and FIGS. 16A-16F, the treatment of cells with IL-1β is used to mimic the peritoneal environment where IL-1β is most frequently measured at higher concentrations in patients with endometriosis (0.1 ng/mL-1 ng/mL) than in unaffected women (<0.01 ng/mL). The use of IL-1β to mimic the pro-inflammatory environment is a common method in cellular assays for endometriosis research. Although the concentrations of IL-1β in these cell cultures are higher than measured in peritoneal fluid, there are multiple other pro-inflammatory cytokines in peritoneal fluid that contribute to the pro-inflammatory environment.

Specifically, 12Z cells were plated at a density of 1×10⁵ cells/well in a 12-well tissue culture plate, serum-starved overnight (17-18 h), pre-treated with vehicle (DMSO) or JNK inhibitor for 30 minutes, then stimulated with 10 ng/ml IL-1β in the presence or absence of the JNK inhibitors for 6 hours. After the 6-hour treatment, mRNA was extracted from the cells using the Qiagen RNEasy mini isolation kit and 1 g of mRNA was reverse transcribed into cDNA using the qScript cDNA Supermix (Quanta Biosciences). cDNA was amplified using SYBR Green Master Mix (Applied Biosystems) with quantitative Real Time PCR (BioRad CFX) using pre-designed and validated primers specific for genes encoding IL6, IL8, PTGS2/COX2, and MMP3 (BioRad). Relative quantification of these specific genes was calculated by the ΔΔCt method, normalizing their abundance relative to the internal housekeeping gene, GAPDH, followed by quantification relative to the vehicle-treated cells. Statistical analyses and visualizations were performed using a One Way ANOVA with a Tukey's multiple comparison post-hoc test on GraphPad Prism version 9.

Bentamapimod and CDD-2628 have similar effects on IL-6 and PTGS2 production, causing concentration-dependent decreases in expression of IL-6 and PTGS2 over a concentration range from 5-25 μM. The concentration of IL-1 (3 added to these cultures is very high, and these results show that in the presence of this extreme environment of inflammation that CDD-2628 is effective at suppressing this inflammatory insult to the tissue. CDD-2628 is more effective than bentamapimod at suppressing IL-8 expression and MMP-3 expression. These results suggest that CDD-2628, a representative inhibitor of the CDD JNK inhibitor series can be more effective at suppressing epithelial mesenchymal transitions (MMP-3) and recruitment of neutrophils to the inflammation site (IL-8). These results also suggest that CDD-2628 and bentamapimod may have equal ability to suppress macrophage and monocyte recruitment to lesions (IL-6, PTGS2). Additional examples of the ability of CDD-JNK inhibitors to suppress EMT compared to bentamapimod are also exemplified by CDD-2575 and CDD-2634.

As further exemplification of the utility of CDD JNK inhibitors, 12Z endometriotic epithelial cells were cultured in the presence of IL-1β and CDD-2728. CDD-2728 effectively suppressed IL-8 production in this in vitro model of inflammation imposed by IL-1β on endometriotic epithelial cells and to a greater extent than bentamapimod. CDD-2728 was also more effective than bentamapimod at suppressing expression of MMP-3 and PTGS2. In this experiment, CDD-2728 and bentamapimod were equally effective at suppressing expression of IL-6.

Another target tissue of JNK inhibitors in the treatment of endometriosis are endometriotic stromal cells. To further exemplify the relevance of the discoveries of novel JNK inhibitors for endometriosis, stromal cells obtained from the endometriotic lesions of a patient with endometriosis have been cultured. The compliant identifier for this patient is Eosis50. These cells represent stromal cells from an ovarian endometrioma. Similar to culture system described elsewhere herein, the inflammatory environment of peritoneal endometriosis was simulated by the addition of IL-1β and increasing concentrations of either bentamapimod or CDD-2728 were added to these stromal cell cultures. Specifically, the cells were cultured at a density of approximately 2×10⁵ cells/well, serum-starved for 17-18 hours, pre-treated with JNK inhibitors for 30 minutes, followed by stimulation with 10 ng/ml IL-1β for 6 hours in the presence or absence of JNK inhibitor. After the 6-hour timepoint, mRNA was extracted, reverse-transcribed using qScript cDNA supermix, and amplified using specific pre-designed and validated primers (IL6, IL8, MMP3, and PTGS2). These results demonstrate the superior efficacy and potency of CDD-2728 compared to bentamapimod on suppression of expression of inflammatory mediators.

To confirm and further support the results described herein, endometriotic stromal cell cultures were established from a second patient. For compliance purposes, this set of cell culture results are identified as Eosis143. These are stromal cells obtained from the lining of an ovarian endometrioma cyst wall. Similar to the observations reported above, CDD-2728 was superior to bentamapimod in efficacy of suppression and potency to achieve suppression of IL-6, PTGS2, IL-8 and MMP-3.

To determine if the responses observed in endometriotic epithelial cells and endometriotic stromal cell cultures were cytotoxic, cell viability was performed in in HepG2 cells. The results highlighted that CDD-2575 reduced ATP consumption by cells as one measure of cytotoxicity. While efficacious, CDD-2575 demonstrated apparent cytotoxicity at concentrations that were lower than those concentrations required to inhibit cytokine expression in epithelial or stromal cells. On the other hand, neither CDD-2624 or CDD-2728 had significant effects on ATP consumption up to 75 M concentrations.

The target product profile seeks to deliver JNK inhibitor as a once daily oral administration. To prioritize compounds for further development, in vitro metabolism of several CDD JNK inhibitors were determined to approximate the plasma half-life and the rate of metabolism of the compounds in vivo. Results from the panel of compounds are shown in Table 10. Among this list of optimized compounds, CDD-2728 held the best potential for achieving the TCP goal for in vivo PK parameters (i.e., once daily oral administration).

TABLE 10
Selected pharmacokinetic data for certain exemplary compounds

| Compound | MLM t_{1/2} (min) | HLM t_{1/2} (min) |
|---|---|---|
| 1 | 7.5 | 36.5 |
| CDD-2574 | | |
| 2 | 31.0 | 7.7 |
| CDD-2325 | | |
| 3 | 68.7 | 188.6 |
| CDD-2346 | | |
| 4 | 33.1 | 30.3 |
| CDD-2347 | | |
| 5 | 126.0 | 119.2 |
| CDD-2488 | | |
| 6 | 116.8 | 408.8 |
| CDD-2575 | | |
| 7 | 40.6 | 98.4 |
| CDD-2628 | | |
| 8 | 15.6 | 12.8 |
| CDD-2491 | | |
| 9 | 120.6 | 122.6 |
| CDD-2387 | | |
| 10 | 16.6 | 56.7 |
| CDD-2482 | | |
| 11 | 30.8 | 71.6 |
| CDD-2461 | | |
| 12 | 10.8 | 33.9 |
| CDD-2486 | | |
| 13 | 28.6 | 74.6 |
| CDD-2635 | | |
| 14 | 16.3 | 16.0 |
| CD-2636 | | |
| 15 | 3.5 | 18.2 |
| CDD-2630 | | |
| 16 | 59.0 | 81.6 |
| CDD-2634 | | |
| 17 | 85.2 | 173.4 |
| CDD-2728 | | |
| 18 | 20.1 | 55.1 |
| CDD-2729 | | |
| 19 | 31.8 | 115.0 |
| CDD-2731 | | |
| 20 | 115.0 | 206 |
| CDD-2855 | | |
| 21 | 55.5 | 225 |
| CDD-2856 | | |
| 22 | 85.0 | 173.0 |
| CDD-2857 | | |
| 23 | 68.9 | 273.0 |
| CDD-2936 | | |
| 24 | 58.0 | 193.0 |
| CDD-2937 | | |
| 25 | 24.0 | 24.3 |
| CDD-2938 | | |
| 26 | 66.7 | 548 |
| CDD-3012 | | |
| 27 | 74.7 | 425.4 |
| CDD-3013 | | |
| 28 | 61.4 | 176.0 |
| CDD-3014 | | |
| 29 | 46.4 | 214.0 |
| CDD-3015 | | |
| 30 | 318 | 293 |
| CDD-3016 | | |
| 31 | 130.6 | 358.0 |
| CDD-3017 | | |
| 32 | 58.8 | 531.0 |
| CDD-3018 | | |
| 33 | 70.3 | 450.1 |
| CDD-3085 | | |
| 33 | 89.8 | 101 |
| CDD-3086 | | |
| 34 | 77.1 | 118 |
| CDD-3087 | | |
| 35 | 124 | 133 |
| CDD-3111 | | |
| 36 | 170 | 99 |
| CDD-3112 | | |
| 37 | 189 | 288 |
| CDD-3113 | | |
| 38 | 86 | 891 |
| CDD-3115 | | |
| 39 | 34 | 48 |
| CDD-3116 | | |
| 40 | 114 | 31 |
| CDD-3117 | | |
| 41 | 144 | 324 |
| CDD-3126 | | |

Based on stability and estimated half-life of compounds exposed to liver microsome in vitro, CDD-2728 was selected for PK evaluation. Similar to the in vitro metabolism predictions, the half-life of CDD-2728 was predicted to be 85.2 minutes (i.e., 1 h 25 min), which was reasonably close to the results obtained by in vivo PK (i.e., 1 h 30 min). To avoid first-pass metabolism, CDD-2728 was also injected into the peritoneal cavity, and the half-life of CDD-2728 was extended to 14 hours, although absorption of compound may have been impacted by the solubility of the compound in the peritoneal cavity.

TABLE 11
Exemplary pharmacokinetic data for
orally administered CDD-2728

| Parameter | Unit | CDD-2728 |
|---|---|---|
| Tₘₐₓ | hr | 0.63 ± 0.25 |
| Cₘₐₓ | μmol/L | 1.51 ± 0.53 |
| T_{1/2} | hr | 1.48 ± 0.32 |
| AUC₀₋ₜ | hr*μmol/L | 2.95 ± 0.95 |
| AUC_{0-∞} | hr*μmol/L | 3.03 ± 0.98 |
| CL/F | mg/(hr*μmol/L)/kg | 17.82 ± 5.48 |
| MRT₀₋ₜ | hr | 1.70 ± 0.34 |
| MRT_{0-∞} | hr | 1.91 ± 0.44 |

TABLE 12
Plasma ALT and AST levels after oral administration
of CDD-2728 (24 h post-dose)

| Mouse No. | Unit | ALT | AST |
|---|---|---|---|
| 1 | IU/L | 43.49 | 69.13 |
| 2 | IU/L | 21.92 | 76.02 |
| 3 | IU/L | 36.24 | 61.79 |
| 4 | IU/L | 30.94 | 41.64 |
| Control-1 | IU/L | 34.56 | 41.81 |
| Control-2 | IU/L | 39.87 | 48.71 |

TABLE 13
Exemplary pharmacokinetic data for intraperitoneal
administered CDD-2728

| Parameter | Unit | CDD-2728 |
|---|---|---|
| Tₘₐₓ | hr | 3.00 ± 1.15 |
| Cₘₐₓ | μmol/L | 0.44 ± 0.19 |
| T_{1/2} | hr | 14.23 ± 8.92 |
| AUC₀₋ₜ | hr*μmol/L | 5.70 ± 2.53 |
| AUC_{0-∞} | hr*μmol/L | 8.03 ± 2.52 |
| CL/F | mg/(hr*μmol/L)/kg | 6.72 ± 2.12 |
| MRT₀₋ₜ | hr | 8.57 ± 1.33 |
| MRT_{0-∞} | hr | 20.49 ± 12.99 |

TABLE 14
Plasma ALT and AST levels after intraperitoneal
administration of CDD-2728 (24 h post-dose)

| Mouse No. | Unit | ALT | AST |
|---|---|---|---|
| 1 | IU/L | 87.34 | 78.94 |
| 2 | IU/L | 29.17 | 38.37 |
| 3 | IU/L | 23.07 | 35.80 |

TABLE 15
Intrinsic metabolic clearance rates in mouse liver
microsomes (MLM) and human liver microsomes (HLM)

| Assay | JQ1 | | Alprazolam | | CDD-3013 | |
|---|---|---|---|---|---|---|
| Time point (min) | 30 | 60 | 30 | 60 | 30 | 60 |
| MLM | 14.7 | 2.8 | 91.6 | 84.0 | 83.4 | 57.3 |
| HLM | 29.6 | 8.1 | 98.8 | 94.7 | 99.4 | 90.7 |

TABLE 16
Predicted half-life in mouse and human plasma
based on intrinsic properties of CDD-3013

| Assay (half-life) | JQ1 | Alprazolam | CDD-3013 |
|---|---|---|---|
| MLM t_{1/2} (min) | 11.6 | 238.5 | 74.7 |
| HLM t_{1/2} (min) | 16.5 | 757.9 | 425.4 |

### Example 7: Identification and Evaluation of JNK Inhibitors Comprising a Piperazine-Based Scaffold

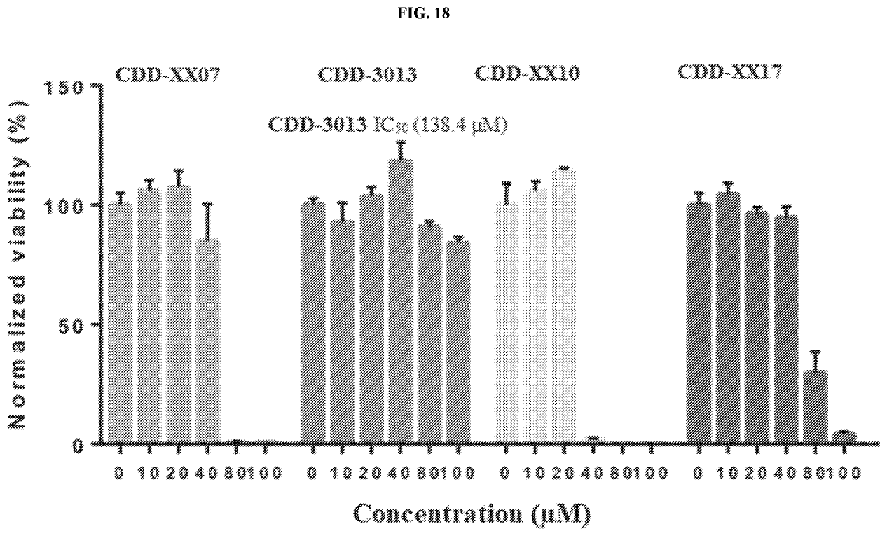

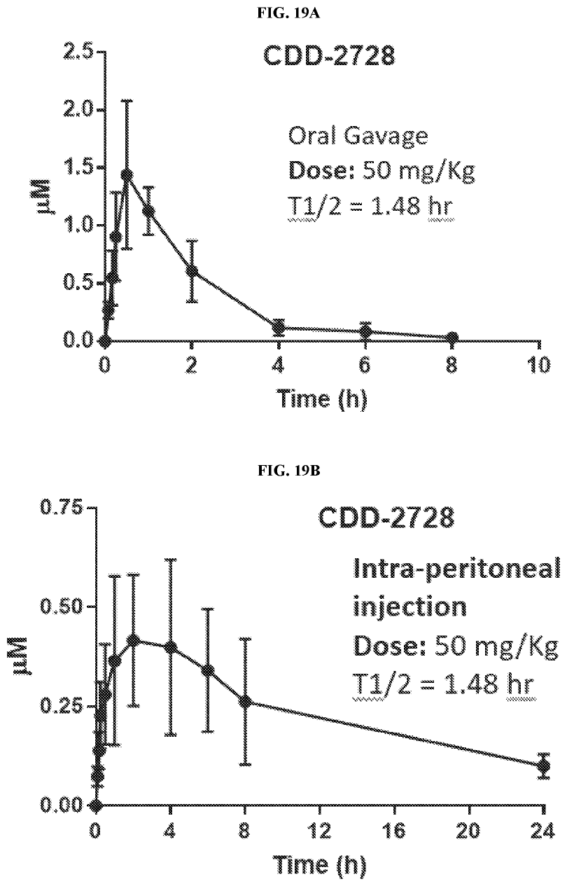

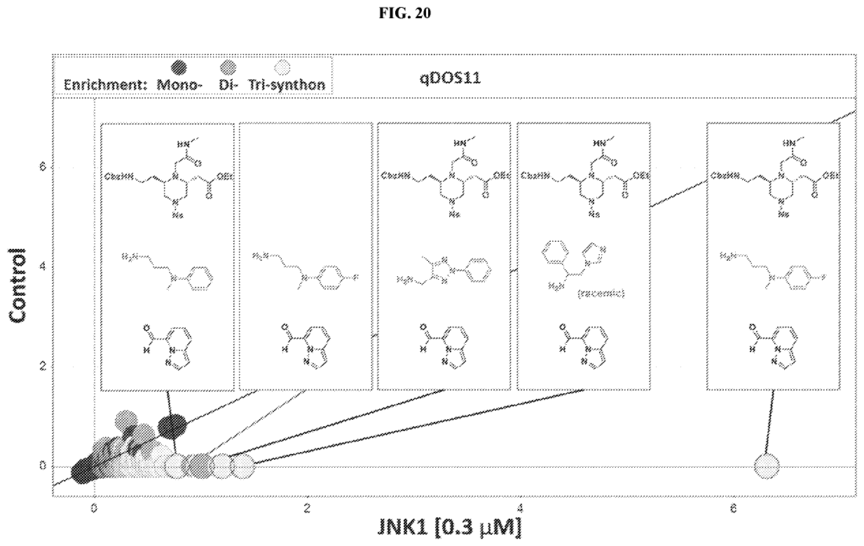

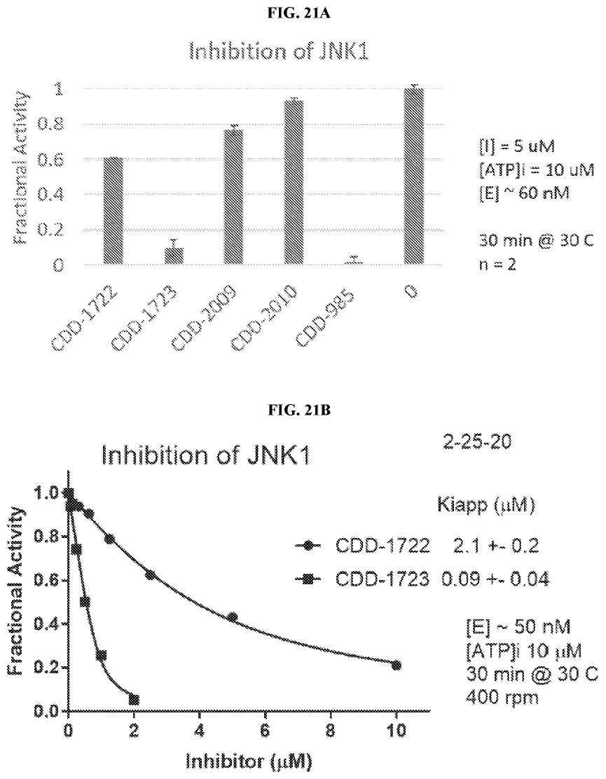

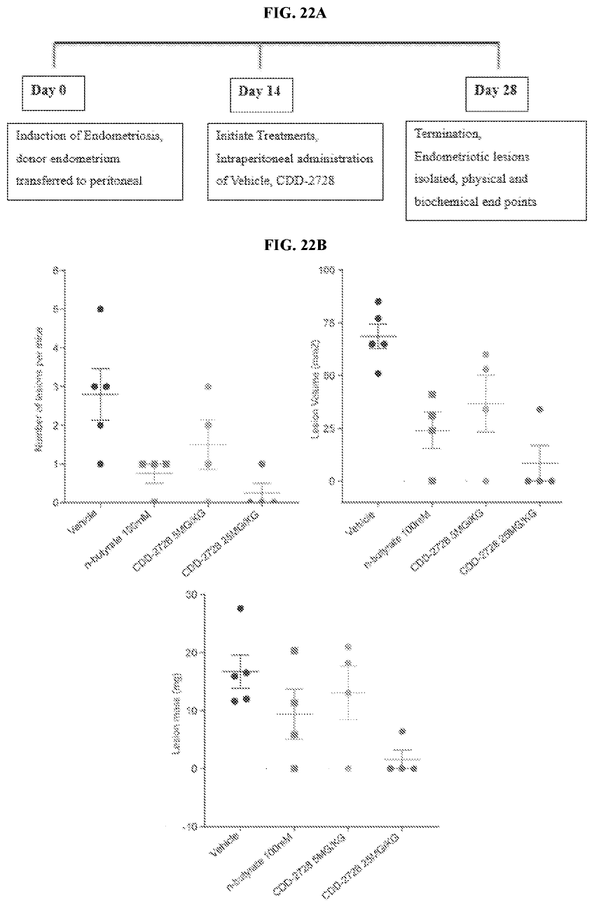

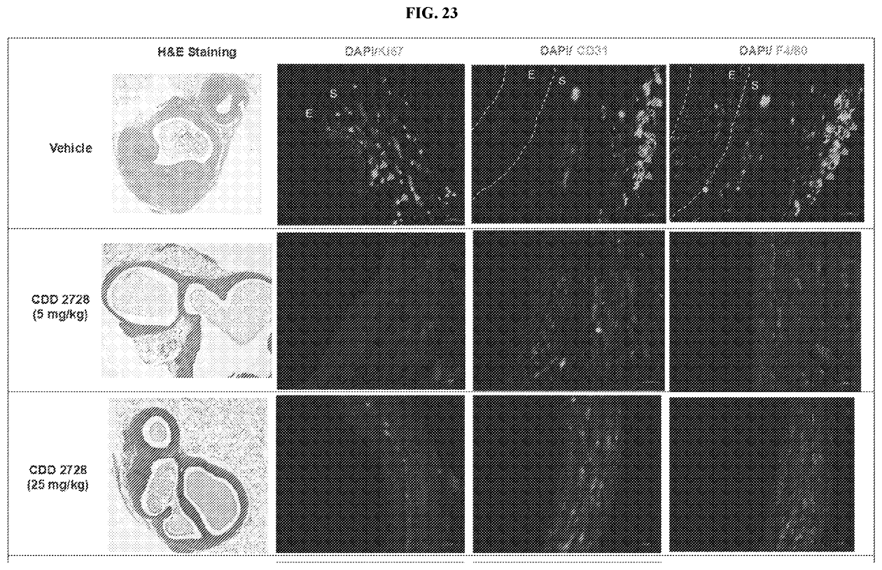

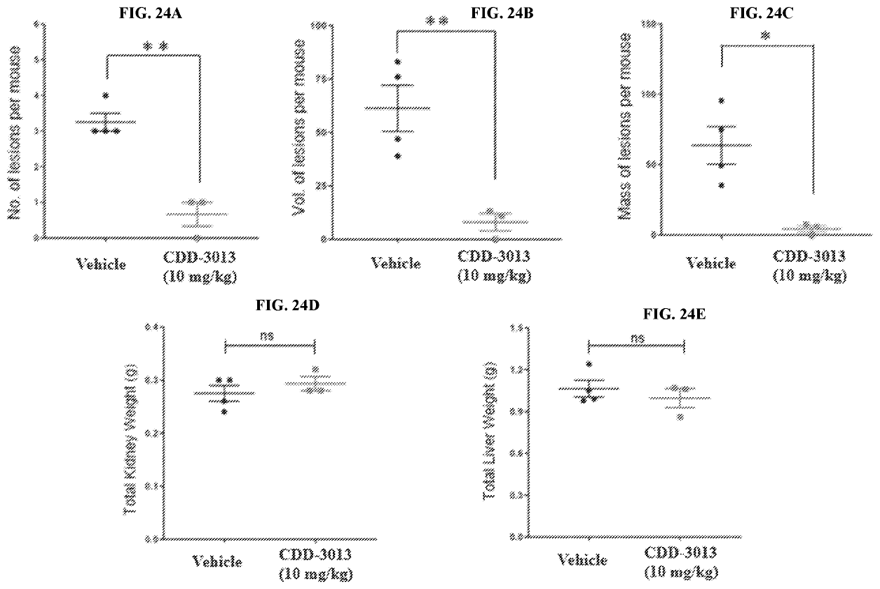

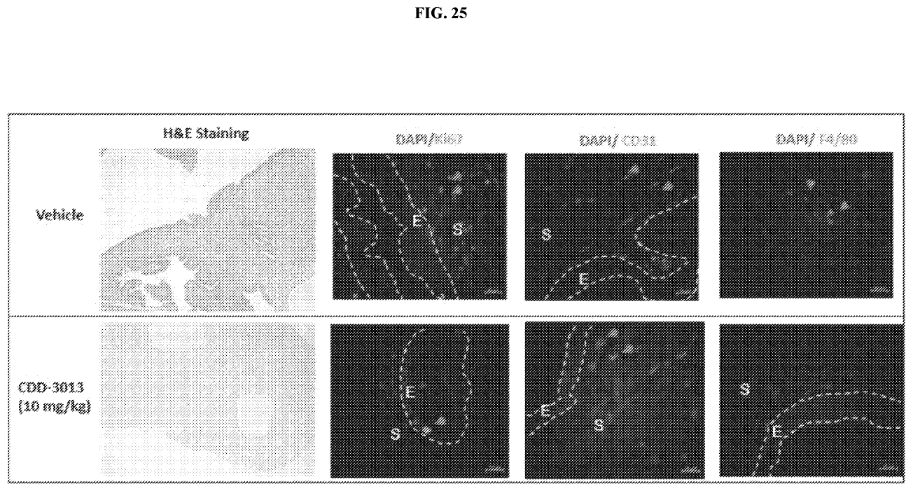

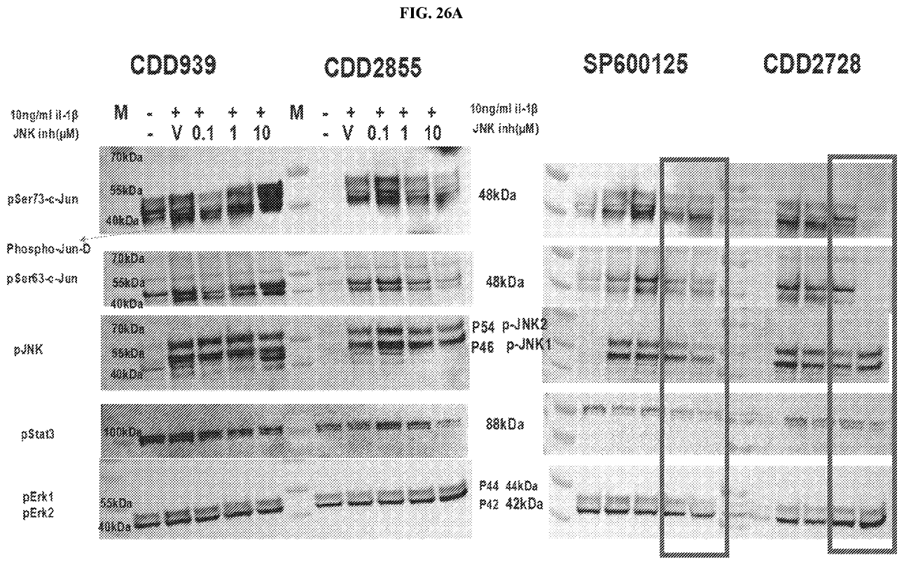

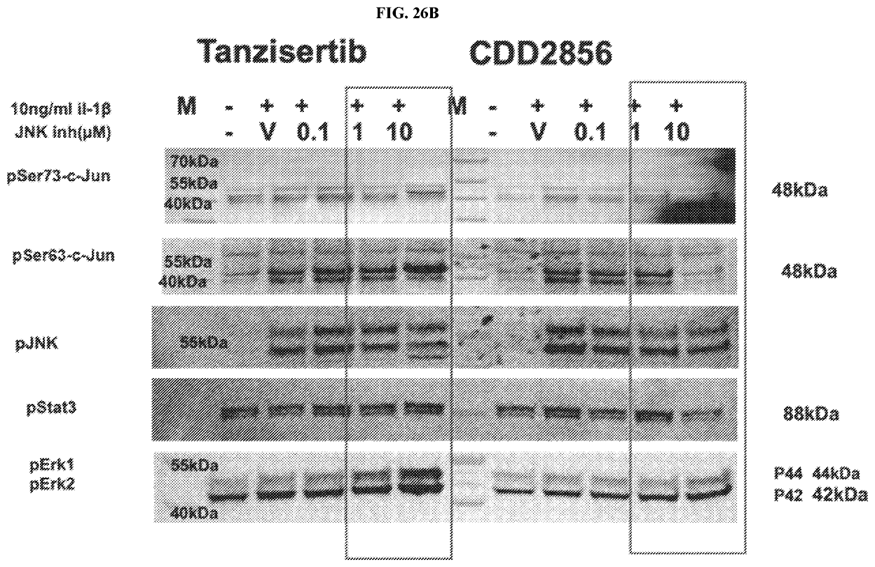

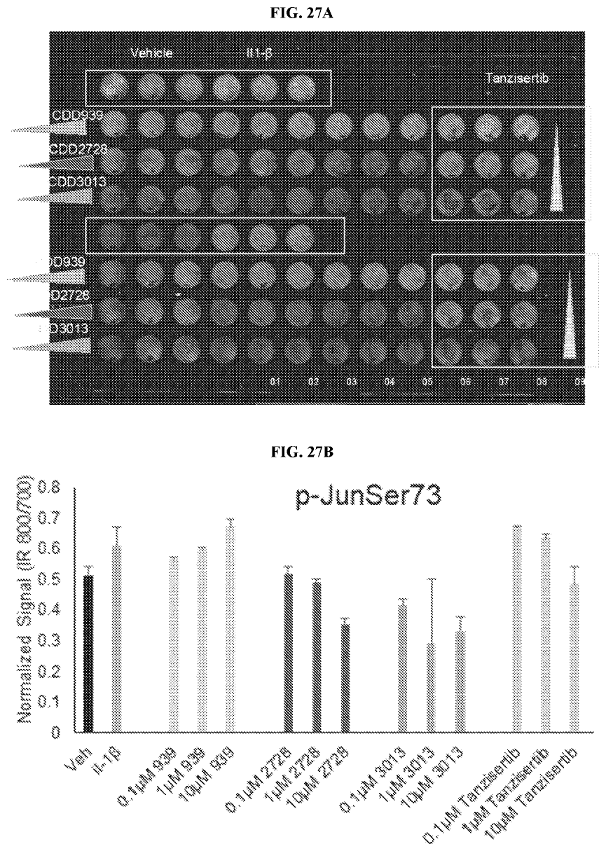

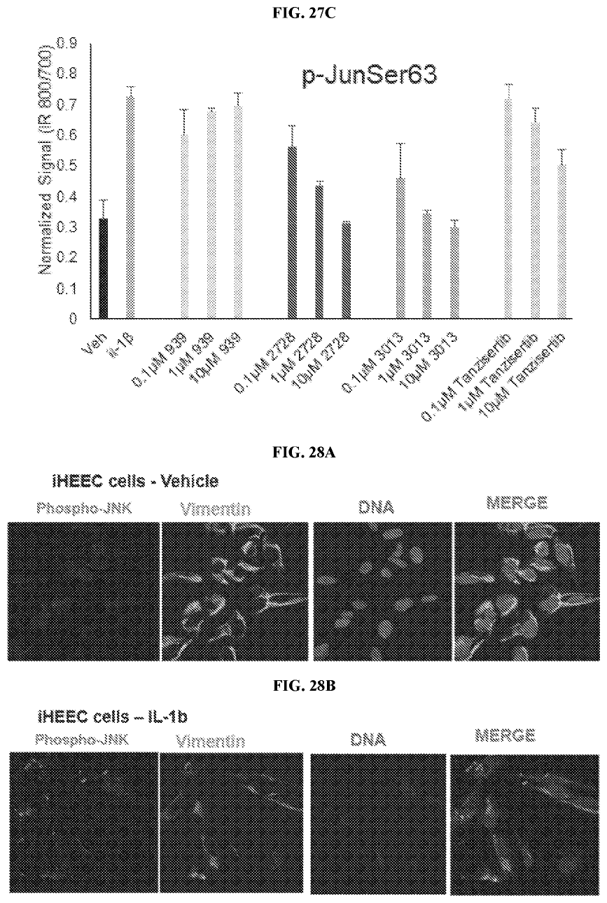

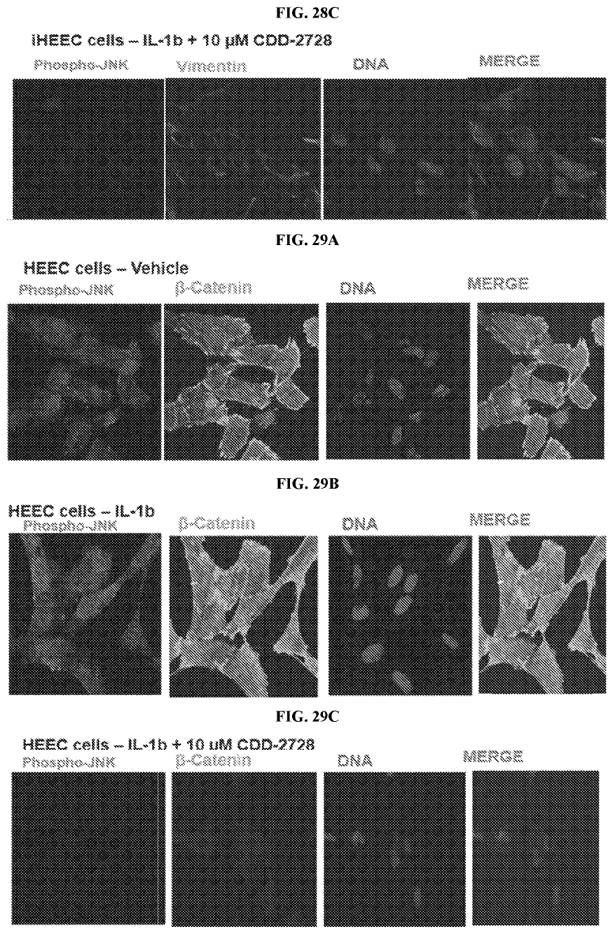

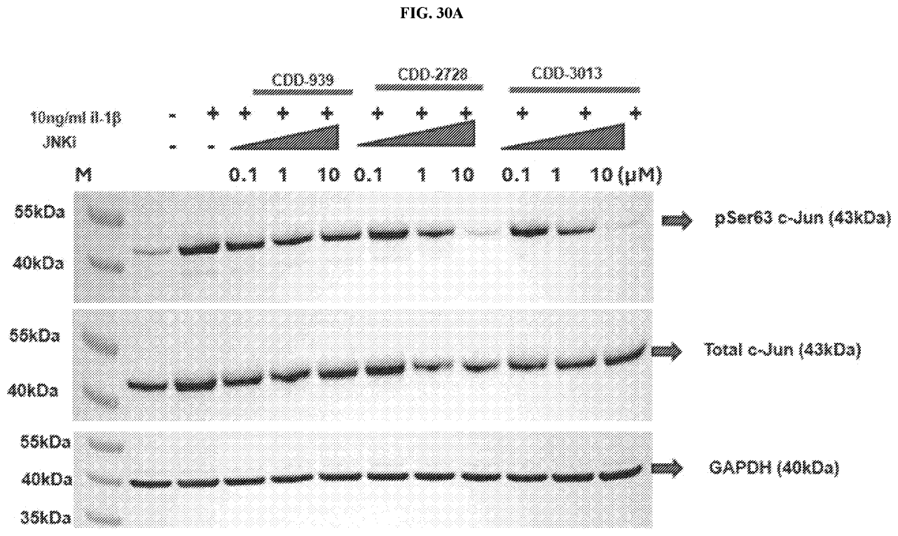

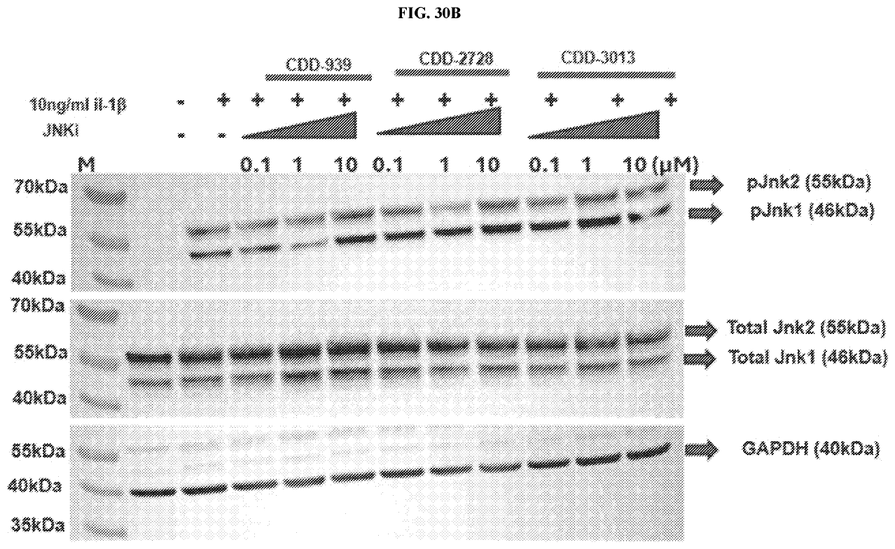

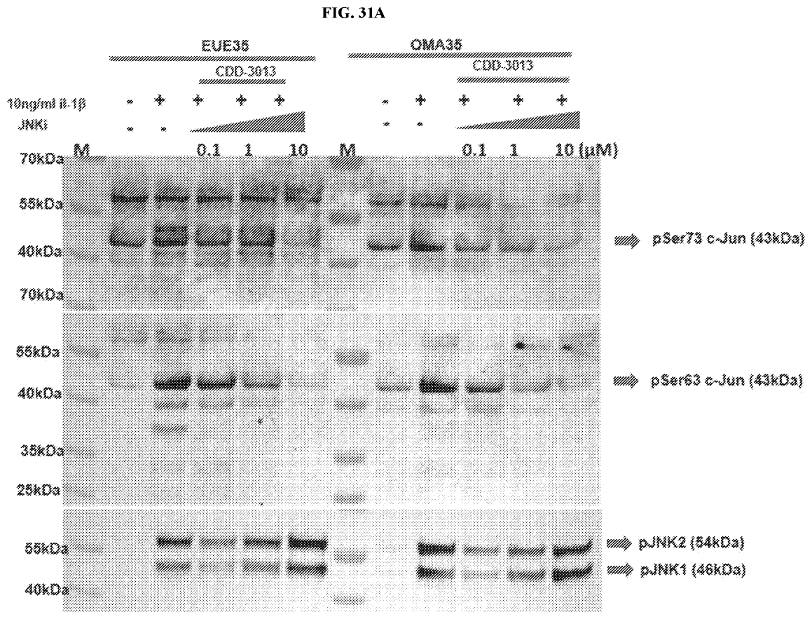

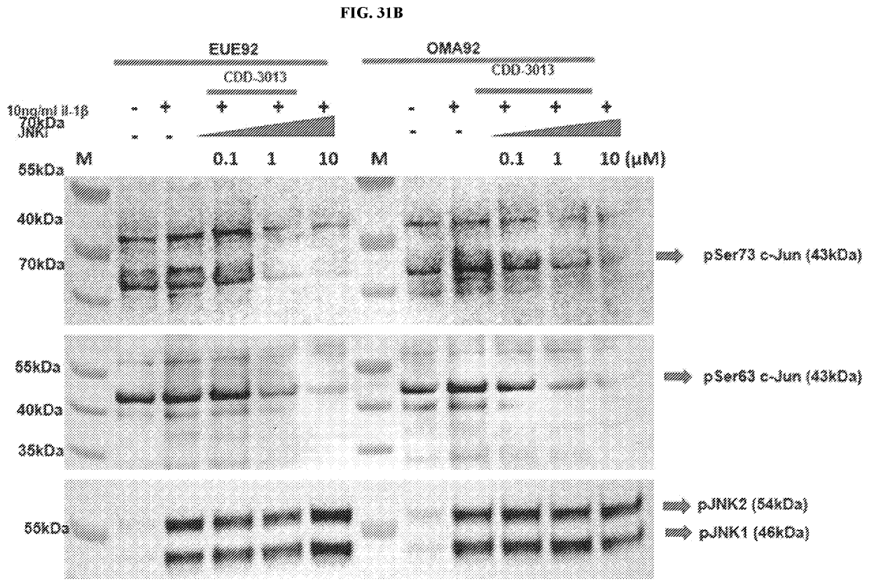

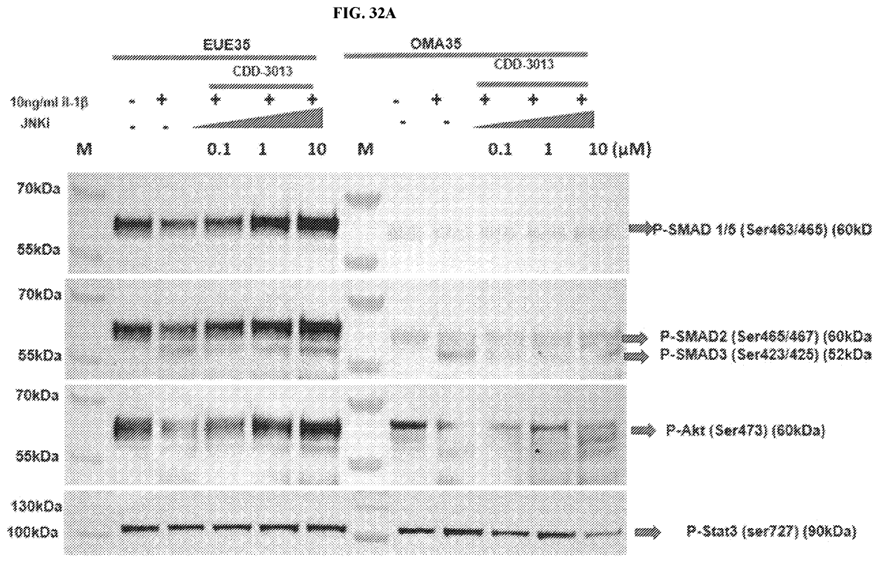

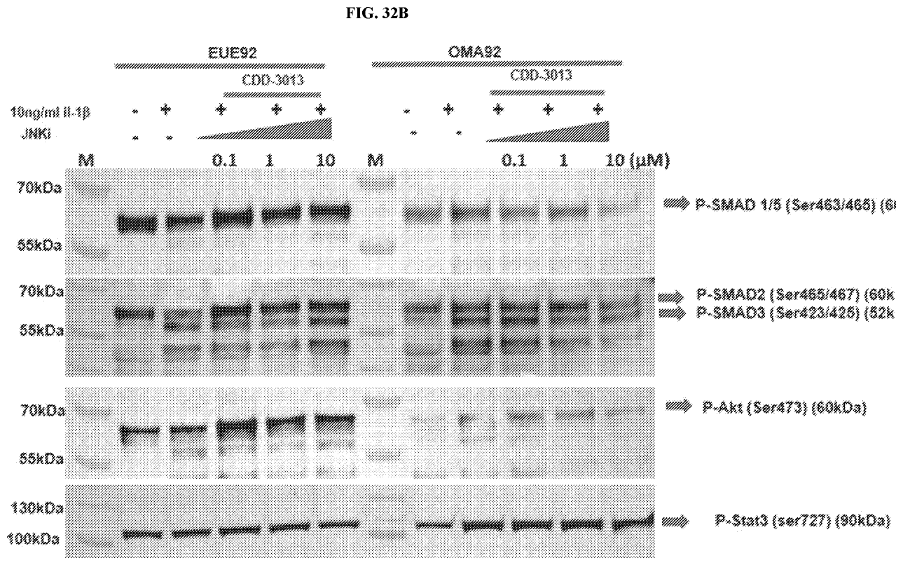

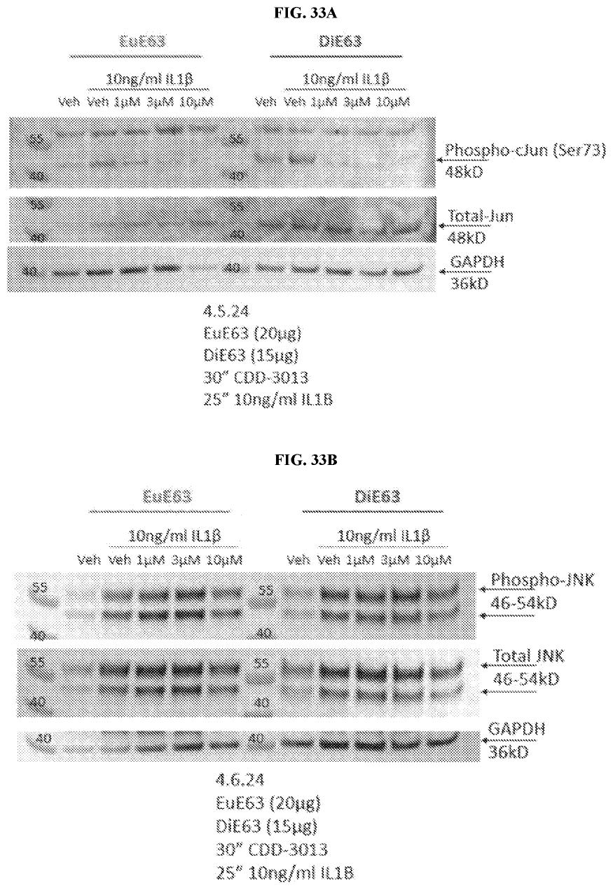

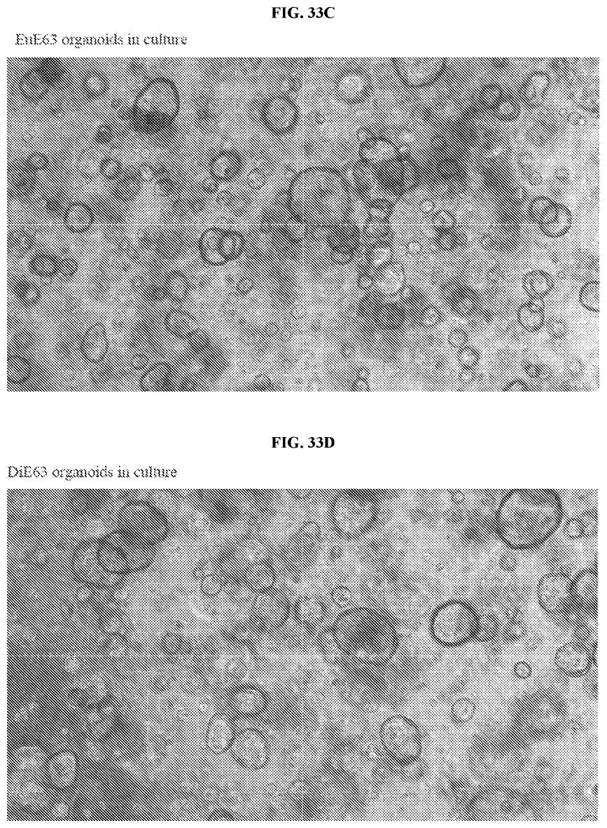

A histidine-tagged recombinant human full length JNK1/JNK3 (Eurofins, USA) was used for the selection against a stereochemically diverse piperazine derived DECL. A pair of affinity selections were conducted where one condition included JNK at 0.3 μM and the other was without protein to serve as a no-target control to identify any non-protein specific enrichment. DEL molecules with affinity to JNK1/JNK3 were retained using nickel nitrilotriacetic (Ni-NTA) magnetic beads which bind polyhistidine. Illumina next-generation sequencing and informatic analysis allowed decoding of the isolated DNA barcode sequences, and, thus, the structures of the enriched drug-like compounds could be identified. Enrichment of binding compounds were measured by a normalized Z-score metric. Comparison of the normalized enrichment of library members in the data sets highlighted the compound series shown in FIG. 18 where reasonable structure-enrichment relationship suggested a promising chemical series for further investigation.

The dose at which CDD-3013 is estimated to cause cytotoxicity based on the experiment depicted in FIG. 18 exceeds 100 μM (i.e., 138.4 μM). For comparison, the IC₅₀ for induction of cytotoxicity response for CDD-xx07 is 48 μM, for CDD-xx10 is 34 μM, and for CDD-xx30 is 72 μM. This indicates that a 138-fold safety margin can be obtained with cell culture and plasma concentrations of 1 μM. and 13.8-fold safety margin is anticipated at plasma concentrations of 10 μM CDD-3013. These results indicate plasma concentrations higher than 10 μM can be tolerated before an adverse effect is observed.

In this affinity selection the scaffold derived from omithine amino acid demonstrated good structure enrichment relationship (SER). Interestingly, one of the enantiomers was enriched better compared to the other. The selectivity identified between enantiomers towards JNK1 on-DNA was subsequently confirmed using off DNA synthesis, following the same synthetic sequence utilized in the DEL build.

Selected chemical features were replaced and/or modified in the off-DNA chemical synthesis, including substitution of the DNA attachment point with a methyl amide. To evaluate the chirality-driven enrichment from the selection experiment, compound CDD-1723 was synthesized, as well as compounds CDD-2010, CDD-1722, CDD-272, and enantiomer CDD-2009.

In certain embodiments, a compound of formula (II) (i.e., piperazine-based compound) can be prepared according to the synthesis described in Scheme 4.

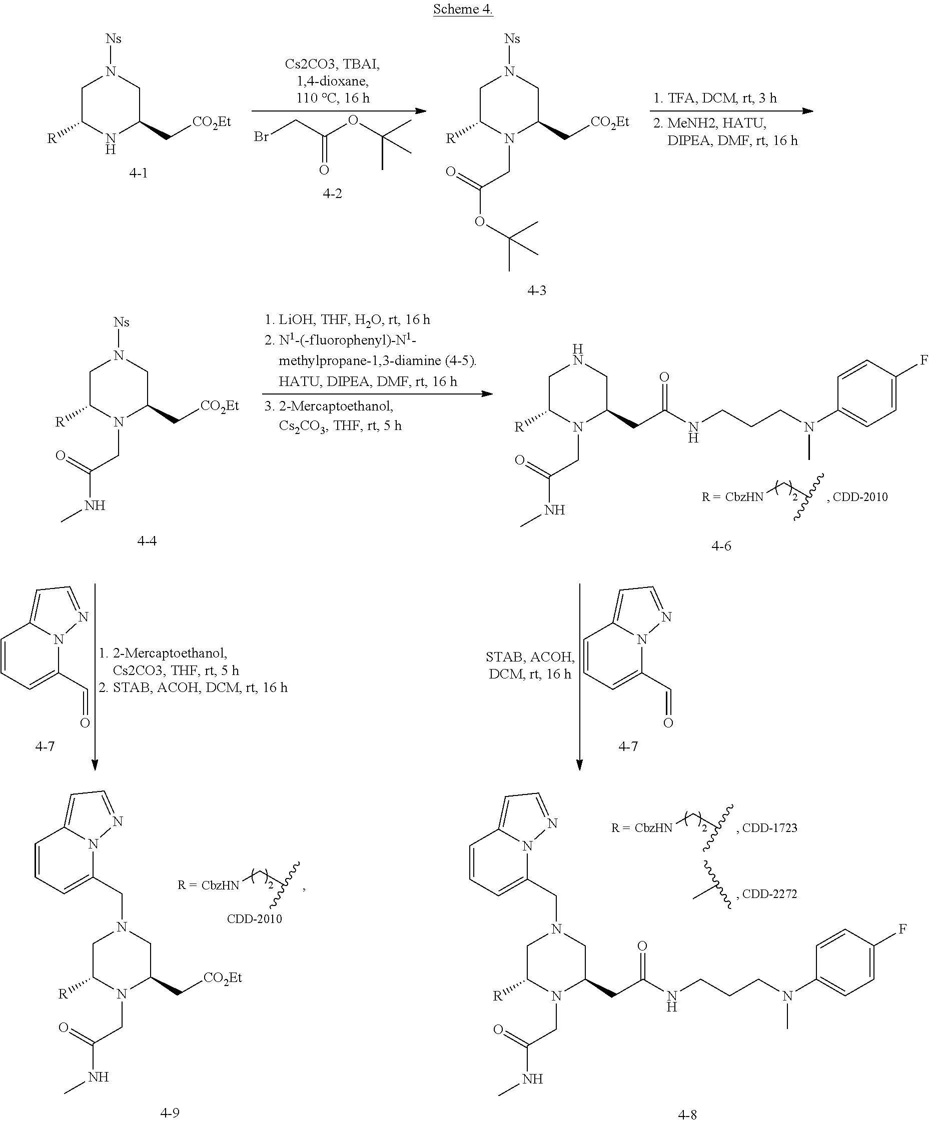

In certain embodiments, compound 4-1 can be alkylated with haloacetate 4-2 to provide compound 4-3, which can be deprotected with a suitable acid (e.g., TFA) and subsequently treated with an amine to provide amide 4-4. Compound 4-4 can be denosylated, and subsequently coupled with amine 4-5 to provide 4-6 (i.e., CDD-2010). Reductive amination of compound 4-6 with aldehyde 4-7 can be performed to afford compound 4-8 (CDD-1723). Alternatively, after denosylation, reductive amination of compound 4-4 with aldehyde 4-7 can be performed to provide compound 4-9 (i.e., CDD-1722).

Selected exemplary compounds were evaluated for JNK1 inhibition, wherein good inhibition of JNK1 was observed for compound CDD-1723, and enantiomeric compound (CDD-2009) was 20-fold less active.

### Enumerated Embodiments

The following exemplary embodiments are provided, the numbering of which is not to be construed as designating levels of importance:

Embodiment 1 provides a compound of Formula (I), or a salt, solvate, stereoisomer, or isotopologue thereof:

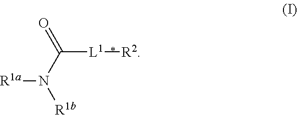

wherein:
R^{1a} is selected from the group consisting of C₁-C₆ alkyl, phenyl, and C₂-C₁₀ heteroaryl, wherein the alkyl, phenyl or heteroaryl is optionally substituted with at least one substituent selected from the group consisting of optionally substituted C₁-C₆ alkyl, halogen, CN, NO₂, OR^{A}, N(R^{A})(R^{B}), C(═O)OR^{A}. C(═O)N(R^{A})(R^{B}), S(═O)₂N(R^{A})(R^{B}), S(═O)N(R^{A})(R^{B}), OC(═O)R^{A}, and N(R^{A})C(═O)R^{B};
R^{1b} is selected from the group consisting of H and optionally substituted C₁-C₆ alkyl, or
   R^{1a} and R^{1b} can combine with the nitrogen atom to which they are bound form an optionally substituted C₂-C₈ heterocycloalkyl;
R² is selected from the group consisting of optionally substituted C₂-C₉ heteroaryl and optionally substituted C₆-C₁₀ aryl;
L¹ is selected from the group consisting of

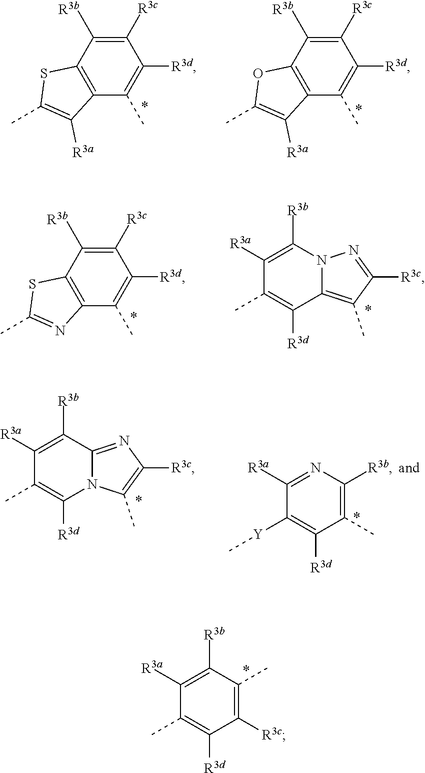

R^{3a}, R^{3b}, R^{3c}, and R^{3d} are each independently selected from the group consisting of H, optionally substituted C₁-C₆ alkyl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D}; and
Y is selected from the group consisting of optionally substituted C₁-C₃ alkylenyl and optionally substituted C₃-C₈ cycloalkylenyl;
R^{A}, R^{B}, R^{C}, and R^{D}, if present, are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl,
   wherein R^{A} and R^{B} or R^{C} and R^{D} can combine with the nitrogen atom to which they are bound form an optionally substituted C₂-C₈ heterocycloalkyl.
   wherein one or more of R^{A} and R^{B} can combine with L to form an optionally substituted C₄-C₅ heterocycloalkyl or C₄-C₆ heteroaryl.

Embodiment 2 provides the compound of Embodiment 1, which is selected from the group consisting of:

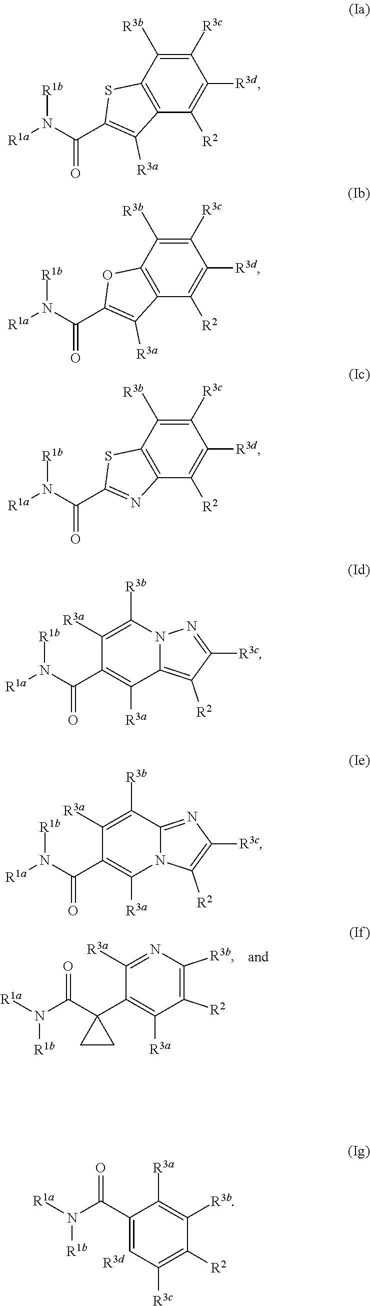

Embodiment 3 provides the compound of Embodiment 1 or 2, wherein R^{1a} is phenyl, pyridinyl, or pyrimidinyl substituted with at least one substituent selected from the group consisting of C₁-C₆ alkyl, C₁-C₆ haloalkyl, F, OH. CN, O(C₁-C₆ alkyl), O(C₁-C₆ haloalkyl), —(CH₂)₁₋₃NH(C₁-C₆ alkyl), —(CH₂)₁₋₃N(C₁-C₆ alkyl)-2, C(═O)NH₂, C(═O)NH(C₁-C₆ alkyl), C(═O)N(C₁-C₆ alkyl)₂, and C(═O)(optionally substituted C₂-C₈ heterocycloalkyl).

Embodiment 4 provides the compound of any one of Embodiments 1-3, wherein R^{1a} is phenyl, pyridinyl, or pyrimidinyl substituted with at least one substituent selected from the group consisting of CH₃, CF₃, OH, OCH₃, OCF₃, F, CN, CH₂NHCH₃, CH₂N(CH₃)₂, C(═O)NH₂, C(═O)NHCH₃, C(═O)N(CH₃)₂, C(═O)(azetidinyl), C(═O)NH(oxiranyl), C(═O)(piperidinyl), C(═O)(4-methylpiperazinyl), C(═O)(morpholinyl), and C(═O)NH(cyclopropylmethyl).

Embodiment 5 provides the compound of any one of Embodiments 1-4, wherein R^{1a} is selected from the group consisting of:

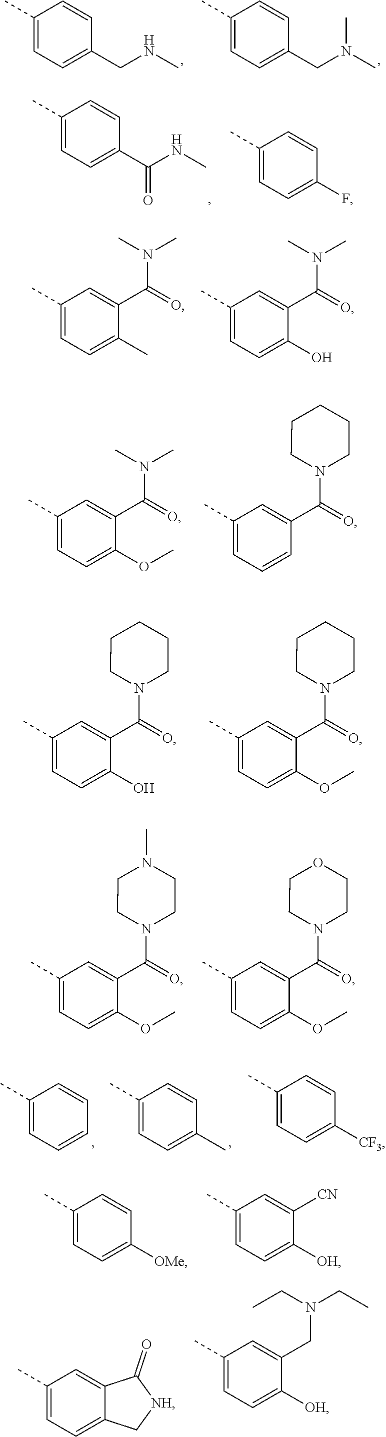

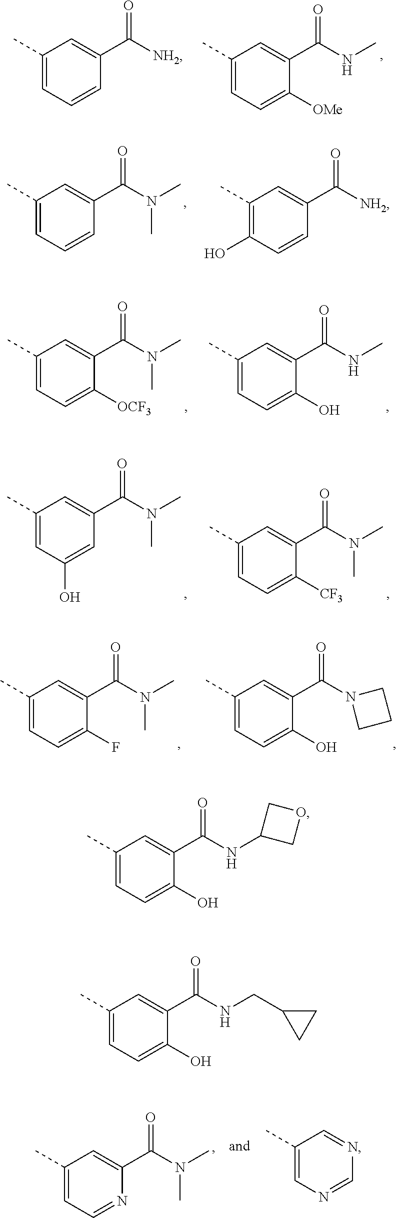

Embodiment 6 provides the compound of Embodiment 1 or 2, wherein R^{1a} is optionally substituted C₁-C₆ alkyl.

Embodiment 7 provides the compound of Embodiment 6, wherein R^{1a} is selected from the group consisting of methyl and CH₂CH₂N(CH₃)₂.

Embodiment 8 provides the compound of Embodiment 1 or 2, wherein R^{1a} and R^{1b} combine with the nitrogen atom to which they are bound to form

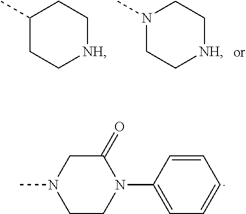

Embodiment 9 provides the compound of any one of Embodiments 1-7 wherein R^{1b} is H.

Embodiment 10 provides the compound of any one of Embodiments 1-9, wherein R² is selected from the group consisting of:

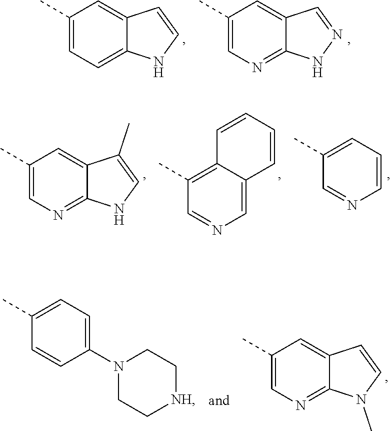

Embodiment 11 provides the compound of any one of Embodiment 1-10, wherein at least one of the following applies:
(a) at least one of R^{3a}, R^{3b}, R^{3c}, and R^{3d} is H;
(b) at least two of R^{3a}, R^{3b}, R^{3c}, and R^{3d} are H:
(c) at least three of R^{3a}, R^{3b}, R^{3c}, and R^{3d} are H; and
(d) each of R^{3a}, R^{3b}, R^{3c}, and R^{3d} are H.

Embodiment 12 provides the compound of any one of Embodiments 1-11, which is selected from the group consisting of
N-methyl-3-(1H-pyrrolo[2,3-b]pyridin-5-yl)pyrazolo[1,5-a]pyridine-5-carboxamide;
3-(isoquinolin-4-yl)-N-(p-tolyl)pyrazolo[1,5-a]pyridine-5-carboxamide;
N-(4-((methylamino)methyl)phenyl)-3-(1H-pyrrolo[2,3-b]pyridin-5-yl)pyrazolo[1,5-a]pyridine-5-carboxamide;
3-(isoquinolin-4-yl)-N-(4-((methylamino)methyl)phenyl)pyrazolo[1,5-a]pyridine-5-carboxamide;
N-(4-((methylamino)methyl)phenyl)-3-(1H-pyrrolo[2,3-b]pyridin-5-yl)imidazo[1,2-a]pyridine-6-carboxamide;
N-(4-((methylamino)methyl)phenyl)-4-(1H-pyrrolo[2,3-b]pyridin-5-yl)benzo[b]thiophene-2-carboxamide;
N-(3-(dimethylcarbamoyl)-4-methoxyphenyl)-4-(1H-pyrrolo[2,3-b]pyridin-5-yl)benzo[b]thiophene-2-carboxamide;
N-(4-methoxy-3-(piperidine-1-carbonyl)phenyl)-4-(1H-pyrrolo[2,3-b]pyridin-5-yl)benzo[b]thiophene-2-carboxamide;
3-(2-aminopyrimidin-5-yl)-N-(4-((methylamino)methyl)phenyl)pyrazolo[1,5-a]pyridine-5-carboxamide;
N-(4-((dimethylamino)methyl)phenyl)-3-(1H-pyrrolo[2,3-b]pyridin-5-yl)pyrazolo[1,5-a]pyridine-5-carboxamide;
N-(4-(methylcarbamoyl)phenyl)-3-(1H-pyrrolo[2,3-b]pyridin-5-yl)pyrazolo[1,5-a]pyridine-5-carboxamide;
N-(4-fluorophenyl)-3-(1H-pyrrolo[2,3-b]pyridin-5-yl)pyrazolo[1,5-a]pyridine-5-carboxamide;
N-(4-methoxy-3-(morpholine-4-carbonyl)phenyl)-4-(1H-pyrrolo[2,3-b]pyridin-5-yl)benzo[b]thiophene-2-carboxamide;
N-(4-methoxy-3-(4-methylpiperazine-1-carbonyl)phenyl)-4-(1H-pyrrolo[2,3-b]pyridin-5-yl)benzo[b]thiophene-2-carboxamide;
N-(3-(piperidine-1-carbonyl)phenyl)-4-(1H-pyrrolo[2,3-b]pyridin-5-yl)benzo[b]thiophene-2-carboxamide;
N-(4-hydroxy-3-(piperidine-1-carbonyl)phenyl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzo[b]thiophene-2-carboxamide;
N-(3-(dimethylcarbamoyl)-4-methylphenyl)-4-(1H-pyrrolo[2,3-b]pyridin-5-yl)benzo[b]thiophene-2-carboxamide;
N-(3-(dimethylcarbamoyl)-4-methoxyphenyl)-4-(3-methyl-1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzofuran-2-carboxamide;
N-(3-(dimethylcarbamoyl)-4-hydroxyphenyl)-4-(1H-pyrrolo[2,3-b]pyridin-5-yl)benzo[d]thiazole-2-carboxamide;
N-(3-(dimethylcarbamoyl)-4-(trifluoromethoxy)phenyl)-4-(1H-pyrrolo[2,3-b]pyridin-5-yl)benzo[b]thiophene-2-carboxamide;
N-(3-carbamoylphenyl)-4-(1H-pyrrolo[2,3-b]pyridin-5-yl)benzo[b]thiophene-2-carboxamide;
N-(3-(dimethylcarbamoyl)-5-hydroxyphenyl)-4-(TH-pyrrolo[2,3-b]pyridin-5-yl)benzo[b]thiophene-2-carboxamide;
N-(5-(dimethylcarbamoyl)-2-hydroxyphenyl)-4-(1H-pyrrolo[2,3-b]pyridin-5-yl)benzo[b]thiophene-2-carboxamide;
4-(4-(1H-pyrrolo[2,3-b]pyridin-5-yl)benzo[b]thiophene-2-carboxamido)-N,N-dimethylpicolinamide;
N-(4-hydroxy-3-(pyrrolidine-1-carbonyl)phenyl)-4-(1H-pyrrolo[2,3-b]pyridin-5-yl)benzo[b]thiophene-2-carboxamide;
N-(3-(azetidine-1-carbonyl)-4-hydroxyphenyl)-4-(1H-pyrrolo[2,3-b]pyridin-5-yl)benzo[b]thiophene-2-carboxamide;
N-(pyrimidin-5-yl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzo[d]thiazole-2-carboxamide;
N-(3-(dimethylcarbamoyl)-4-hydroxyphenyl)-4-(3-methyl-1H-pyrrolo[2,3-b]pyridin-5-yl)benzo[b]thiophene-2-carboxamide;
N-(3-(dimethylcarbamoyl)-4-hydroxyphenyl)-4-(1H-pyrazolo[3,4-b]pyridin-5-yl)benzo[b]thiophene-2-carboxamide;
N-(2-(dimethylamino)ethyl)-4-(TH-pyrrolo[2,3-b]pyridin-5-yl)benzo[b]thiophene-2-carboxamide;
N-(3-((2-(dimethylamino)ethyl)carbamoyl)-4-hydroxyphenyl)-4-(1H-pyrrolo[2,3-b]pyridin-5-yl)benzo[b]thiophene-2-carboxamide;
N-(4-hydroxy-3-(methylcarbamoyl)phenyl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)thieno[2,3-c]pyridine-2-carboxamide;
N-(4-hydroxy-3-(oxetan-3-ylcarbamoyl 1)phenyl)-4-(1H-pyrrolo[2,3-b]pyridin-5-yl)benzo[b]thiophene-2-carboxamide;
N-(3-((cyclopropylmethyl)carbamoyl)-4-hydroxyphenyl)-4-(1H-pyrrolo[2,3-b]pyridin-5-yl)benzo[b]thiophene-2-carboxamide;
N-(3-oxoisoindolin-5-yl)-4-(1H-pyrrolo[2,3-b]pyridin-5-yl)benzo[b]thiophene-2-carboxamide;
4-(4-(1H-pyrrolo[2,3-b]pyridin-5-yl)benzo[b]thiophene-2-carbonyl)-1-phenylpiperazin-2-one;
N-(3-((diethylamino)methyl)-4-hydroxyphenyl)-4-(1H-pyrrolo[2,3-b]pyridin-5-yl)benzo[b]thiophene-2-carboxamide;
N-(3-cyano-4-hydroxyphenyl)-4-(1H-pyrrolo[2,3-b]pyridin-5-yl)benzo[b]thiophene-2-carboxamide;
N-phenyl-3-(1H-pyrrolo[2,3-b]pyridin-5-yl)pyrazolo[1,5-a]pyridine-5-carboxamide;
3-(1H-pyrrolo[2,3-b]pyridin-5-yl)-N-(p-tolyl)pyrazolo[1,5-a]pyridine-5-carboxamide; 3-(pyridin-4-yl)-N-(p-tolyl)pyrazolo[1,5-a]pyridine-5-carboxamide;
3-(1H-indol-5-yl)-N-(p-tolyl)pyrazolo[1,5-a]pyridine-5-carboxamide;
3-(1H-indazol-5-yl)-N-(4-((methylamino)methyl)phenyl)pyrazolo[1,5-a]pyridine-5-carboxamide;
N-(4-((methylamino)methyl)phenyl)-3-(4-(piperazin-1-yl)phenyl)pyrazolo[1,5-a]pyridine-5-carboxamide;
3-(1-methyl-1H-pyrrolo[2,3-b]pyridin-5-yl)-N-(4-((methylamino)methyl)phenyl)pyrazolo[1,5-a]pyridine-5-carboxamide;
3-(1H-pyrrolo[2,3-b]pyridin-5-yl)-N-(4-(trifluoromethyl)phenyl)pyrazolo[1,5-a]pyridine-5-carboxamide;
(3-(1H-pyrrolo[2,3-b]pyridin-5-yl)pyrazolo[1,5-a]pyridin-5-yl)(piperidin-1-yl)methanone;
(3-(1H-pyrrolo[2,3-b]pyridin-5-yl)pyrazolo[1,5-a]pyridin-5-yl)(piperazin-1-yl)methanone;
1-(5-(1H-pyrrolo[2,3-b]pyridin-5-yl)pyridin-3-yl)-N-(4 ((methylamino)methyl)phenyl)cyclopropane-1-carboxamide;
N-(4-((methylamino)methyl)phenyl)-4-(1H-pyrrolo[2,3-b]pyridin-5-yl)benzamide;
N-methyl-4-(1H-pyrrolo[2,3-b]pyridin-5-yl)benzo[b]thiophene-2-carboxamide;
N-phenyl-4-(1H-pyrrolo[2,3-b]pyridin-5-yl)benzo[b]thiophene-2-carboxamide;
N-(4-methoxyphenyl)-4-(1H-pyrrolo[2,3-b]pyridin-5-yl)benzo[b]thiophene-2-carboxamide;
N-(4-methoxy-3-(methylcarbamoyl)phenyl)-4-(1H-pyrrolo[2,3-b]pyridin-5-yl)benzo[b]thiophene-2-carboxamide;
N-(4-methoxy-3-(4-methylpiperazine-1-carbonyl)phenyl)-4-(1H-pyrrolo[2,3-b]pyridin-5-yl)benzo[b]thiophene-2-carboxamide;
N-(3-(dimethylcarbamoyl)phenyl)-4-(1H-pyrrolo[2,3-b]pyridin-5-yl)benzo[b]thiophene-2-carboxamide;
N-(3-(dimethylcarbamoyl)-4-(trifluoromethyl)phenyl)-4-(1H-pyrrolo[2,3-b]pyridin-5-yl)benzo[b]thiophene-2-carboxamide; and
N-(3-(dimethylcarbamoyl)-4-fluorophenyl)-4-(1H-pyrrolo[2,3-b]pyridin-5-yl)benzo[b]thiophene-2-carboxamide.

Embodiment 13 provides a compound of Formula (II), or a salt, solvate, stereoisomer, or isotopologue thereof:

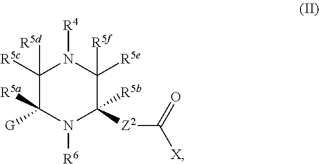

wherein:
R⁴ is selected from the group consisting of H, optionally substituted C₁-C₆ alkyl, optionally substituted C₃-C₈ cycloalkyl, optionally substituted C₂-C₆ heterocycloalkyl, optionally substituted C₇-C₁₂ aralkyl, optionally substituted C₃-C₁₂ heteroaralkyl, optionally substituted C₆-C₁₀ aryl, and optionally substituted C₂-C₁₂ heteroaryl;
R^{5a}, R^{5b}, R^{5c}, R^{5d}, R^{5e}, and R^{5f} are each independently selected from the group consisting of H, optionally substituted C₁-C₆ alkyl, halogen, OR^{E}, and N(R^{E})(R^{F}),
R⁶ is —(CH₂)₁₋₃C(═O)N(R^{G})(R^{H});
R^{7a} is selected from the group consisting of H and C(═O)OR¹
R^{7b} is H;
X is selected from the group consisting of OR^{J} and

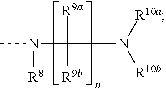

G is selected from the group consisting of optionally substituted C₁-C₆ alkyl and —Z¹—N(R^{7a})(R^{7b});
Z¹ and Z² are each independently-(optionally substituted C₁-C₆ alkylenyl)-:
R⁸ is selected from the group consisting of H and optionally substituted C₁-C₆ alkyl;
R^{9a} and R^{9b} are each independently selected from the group consisting of H and optionally substituted C₁-C₆ alkyl:
R^{10a} and R^{10b} are each independently selected from the group consisting of H, optionally substituted C₁-C₆ alkyl, and optionally substituted C₆-C₁₀ aryl;
n is an integer selected from the group consisting of 1, 2, 3, 4, and 5; and
R^{E}, R^{F}, R^{G}, R^{H}, R^{I}, and R^{J} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

Embodiment 14 provides the compound of Embodiment 13, wherein R⁴ is selected from the group consisting of H and

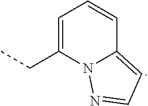

Embodiment 15 provides the compound of Embodiment 13 or 14, wherein at least one of the following applies:
(a) at least one of R^{5a}, R^{5b}, R^{5c}, R^{5d}, R^{5e}, and R^{5f} is H;
(b) at least two of R^{5a}, R^{5b}, R^{5c}, R^{5d}, R^{5e}, and R^{5f} are H;
(c) at least three of R^{5a}, R^{5b}, R^{5c}, R^{5d}, R^{5e}, and R^{5f} are H;
(d) at least four of R^{5a}, R^{5b}, R^{5c}, R^{5d}, R^{5e}, and R^{5f} are H;
(e) at least five of R^{5a}, R^{5b}, R^{5c}, R^{5d}, R^{5e}, and R^{5f} are H; and
(f) each of R^{5a}, R^{5b}, R^{5c}, R^{5d}, R^{5e}, and R^{5f} are H.

Embodiment 16 provides the compound of any one of Embodiments 13-15, wherein R⁶ is —CH₂C(═O)NHCH₃.

Embodiment 17 provides the compound of any one of Embodiments 13-16, wherein G is CH₃.

Embodiment 18 provides the compound of any one of Embodiments 13-16, wherein G is —Z¹—N(R^{7a})(R_{7b}).

Embodiment 19 provides the compound of any one of Embodiments 13-16 and 18, wherein R^{7a} is selected from the group consisting of H and

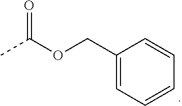

Embodiment 20 provides the compound of any one of Embodiments 13-16 and 18-19, wherein R^{7b} is H.

Embodiment 21 provides the compound of any one of Embodiments 13-20, wherein X is OEt.

Embodiment 22 provides the compound of any one of Embodiments 13-20, wherein X is

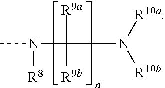

Embodiment 23 provides the compound of anyone of Embodiments 13-20 and 22, wherein each occurrence of R^{9a} and R^{9b} are independently H.

Embodiment 24 provides the compound of any one of Embodiments 13-20 and 22-23, wherein n is 3.

Embodiment 25 provides the compound of any one of Embodiments 13-20 and 22-23, wherein R^{10a} and R^{10b} are each independently selected from the group consisting of 4-fluorophenyl and methyl.

Embodiment 26 provides the compound of any one of Embodiments 13-20 and 22-25, wherein X is

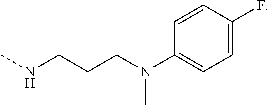

Embodiment 27 provides the compound of any one of Embodiments 13-26, wherein Z¹ is —CH₂CH₂CH₂—.

Embodiment 28 provides the compound of any one of Embodiments 13-27, wherein Z² is —CH₂—.

Embodiment 29 provides the compound of any one of Embodiments 13-28, which is selected from the group consisting of:
benzyl (2-((2R,6R)-6-(2-((3-((4-fluorophenyl)(methyl)amino)propyl)amino)-2-oxoethyl)-1-(2-(methylamino)-2-oxoethyl)-4-(pyrazolo[1,5-a]pyridin-7-ylmethyl)piperazin-2-yl)ethyl)carbamate:
ethyl 2-((2R,6R)-6-(2-(((benzyloxy)carbonyl)amino)ethyl)-1-(2-(methylamino)-2-oxoethyl)-4-(pyrazolo[1,5-a]pyridin-7-ylmethyl)piperazin-2-yl)acetate;
benzyl (2-((2R,6R)-6-(2-((3-((4-fluorophenyl)(methyl)amino)propyl)amino)-2-oxoethyl)-1-(2-(methylamino)-2-oxoethyl)piperazin-2-yl)ethyl)carbamate;
N-(3-((4-fluorophenyl)(methyl)amino)propyl)-2-((2R,6R)-6-methyl-1-(2-(methylamino)-2-oxoethyl)piperazin-2-yl)acetamide.

Embodiment 30 provides the pharmaceutical composition comprising at least one compound of any one of Embodiments 1-29 and a pharmaceutically acceptable carrier.

Embodiment 31 provides the pharmaceutical composition of Embodiment 30, further comprising at least one therapeutically effective agent.

Embodiment 32 provides a method of treating, preventing, and/or ameliorating an inflammatory disease in a subject, the method comprising administering to the subject a therapeutically effective amount of at least one compound of any one of Embodiments 1-29 and/or the pharmaceutical composition of Embodiment 30 or 31.

Embodiment 33 provides the method of Embodiment 32, wherein JNK1 is inhibited at a similar or greater rate of inhibition than JNK2.

Embodiment 34 provides the method of Embodiment 32 or 33, wherein JNK1 is inhibited at a similar or greater rate of inhibition than JNK3.

Embodiment 35 provides the method of any one of Embodiments 32-34, wherein the inflammatory disease is a non-central nervous system (CNS) inflammatory disease.

Embodiment 36 provides the method of any one of Embodiments 32-35, wherein the inflammatory disease is at least one selected from the group consisting of endometriosis, arthritis, pulmonary fibrosis, cancer, type 1 diabetes, and type 2 diabetes.

Embodiment 37 provides the method of any one of Embodiments 32-36, wherein the inflammatory disease is endometriosis.

Embodiment 38 provides the method of any one of Embodiments 32-37, wherein progesterone insensitivity in the subject is reduced and/or eliminated.

Embodiment 39 provides the method of any one of Embodiments 32-38, wherein pain associated with endometriosis is reduced and/or eliminated.

Embodiment 40 provides the method of any one of Embodiments 32-34 and 38, wherein the inflammatory disease is selected from the group consisting of Alzheimer's disease, Parkinson's disease, and amyotrophic lateral sclerosis.

Embodiment 41 provides the method of any one of Embodiments 32-34, 38, and 40, wherein the subject is further administered progesterone.

Embodiment 42 provides the method of any one of Embodiments 32-41, wherein normal reproductive function is maintained in the subject.

Embodiment 43 provides the method of any one of Embodiments 32-42, wherein the method further comprises detecting the inflammatory disease or disorder in the subject.

Embodiment 44 provides the method of claim 43, wherein the detecting comprises administration of a suitable diagnostic method.

Embodiment 45 provides the method of any one of Embodiments 32-44, wherein the subject is a mammal.

Embodiment 46 provides the method of Embodiment 45, wherein the mammal is a human.

Embodiment 47 provides the method of Embodiment 46, wherein the human is a female.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of Formula (I), or a salt, solvate, stereoisomer, or isotopologue thereof: 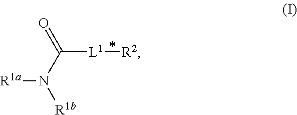
wherein:
R^{1a} is selected from the group consisting of C₁-C₆ alkyl, phenyl, and C₂-C₁₀ heteroaryl, wherein the alkyl, phenyl or heteroaryl is optionally substituted with at least one substituent selected from the group consisting of optionally substituted C₁-C₆ alkyl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B} OC(═O)R^{A}, and N(R^{A})C(═O)R^{B};
R^{1b} is selected from the group consisting of H and optionally substituted C₁-C₆ alkyl, or
R^{1a} and R^{1b} can combine with the nitrogen atom to which they are bound form an optionally substituted C₂-C₈ heterocycloalkyl;
R² is selected from the group consisting of optionally substituted C₂-C₉ heteroaryl and optionally substituted C₆-C₁₀ aryl;
L¹ is selected from the group consisting of 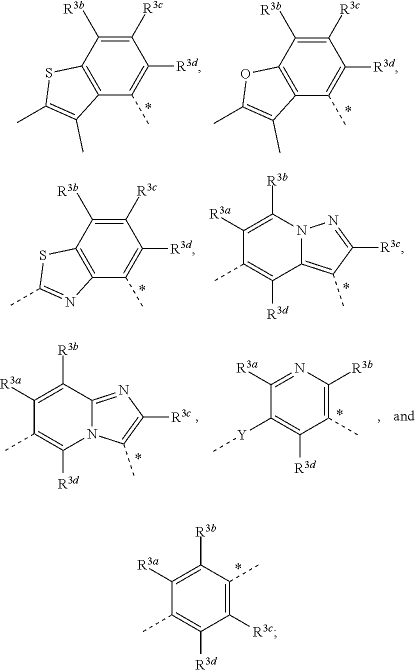
R^{3a}, R^{3b}, R^{3c}, and R^{3d} are each independently selected from the group consisting of H, optionally substituted C₁-C₆ alkyl, halogen, CN, NO₂, OR^{C}, N(R^{C})(R^{D}), C(═O)OR^{C}, C(═O)N(R^{C})(R^{D}), S(═O)₂N(R^{C})(R^{D}), S(═O)N(R^{C})(R^{D}), OC(═O)R^{C}, and N(R^{C})C(═O)R^{D}; and
Y is selected from the group consisting of optionally substituted C₁-C₃ alkylenyl and optionally substituted C₃-C₈ cycloalkylenyl;
R^{A}, R^{B}, R^{C}, and R^{D}, if present, are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl,
wherein R^{A} and R^{B} or R^{C} and R^{D} can combine with the nitrogen atom to which they are bound form an optionally substituted C₂-C₈ heterocycloalkyl,
wherein one or more of R^{A} and R^{B} can combine with L to form an optionally substituted C₄-C₈ heterocycloalkyl or C₄-C₆ heteroaryl.

2. The compound of claim 1, which is selected from the group consisting of:

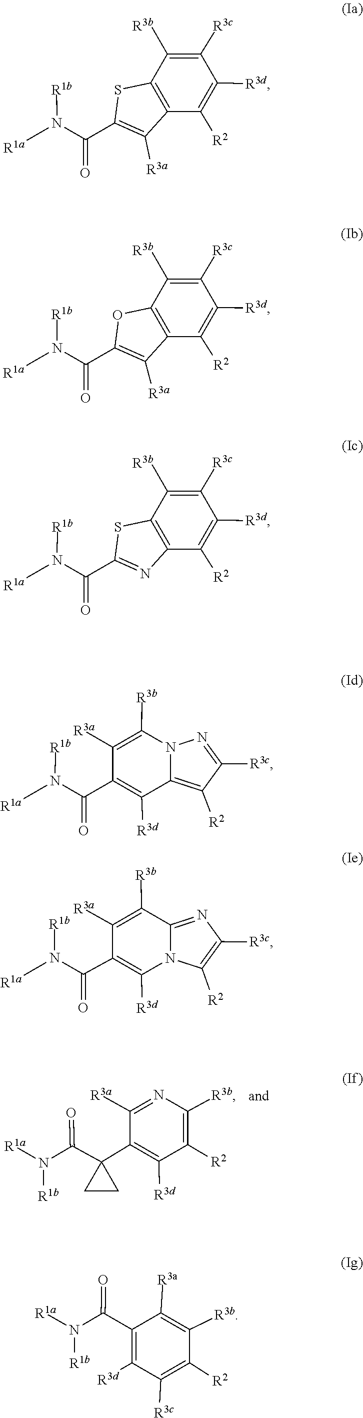

3. The compound of claim 1, wherein one of the following applies:
(a) R^{1a} is phenyl, pyridinyl, or pyrimidinyl substituted with at least one substituent selected from the group consisting of C₁-C₆ alkyl, C₁-C₆ haloalkyl, F, OH, CN, O(C₁-C₆ alkyl), O(C₁-C₆ haloalkyl), —(CH₂)₁₋₃NH(C₁-C₆ alkyl), —(CH₂)₁₋₃N(C₁-C₆ alkyl)-2, C(═O)NH₂, C(═O)NH(C₁-C₆ alkyl), C(═O)N(C₁-C₆ alkyl)₂, and C(═O)(optionally substituted C₂-C₈ heterocycloalkyl); or
(b) R^{1a} is optionally substituted C₁-C₆ alkyl.

4. (canceled)

5. The compound of claim 1, wherein one of the following applies:
(a) R^{1a} is selected from the group consisting of: 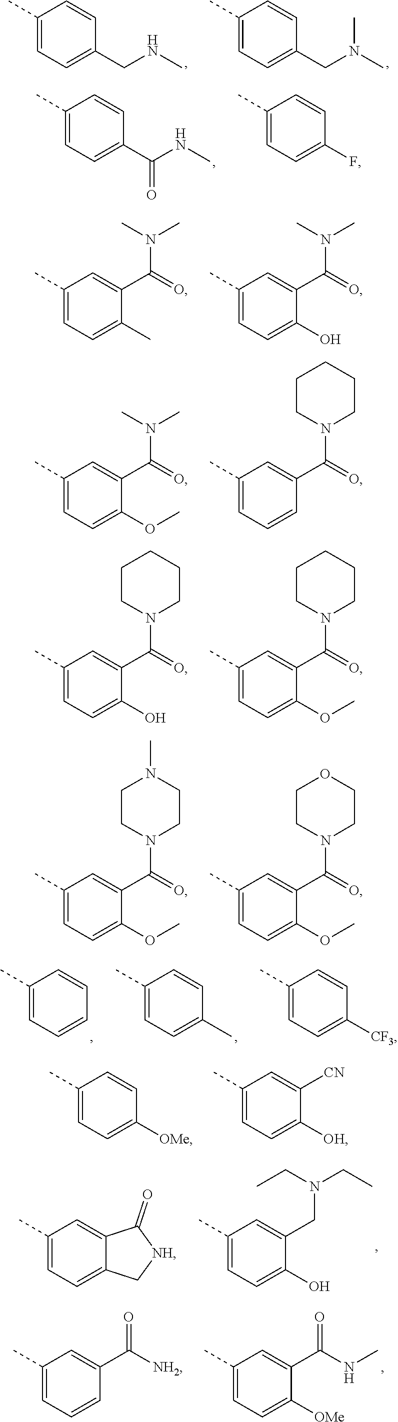 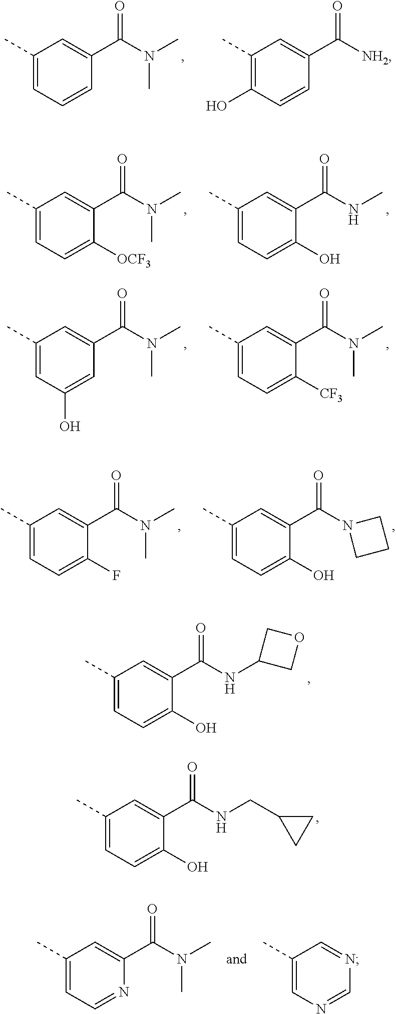
(b) R^{1a} is selected from the group consisting of methyl and CH₂CH₂N(CH₃)₂; or
(c) R^{1a} and R^{1b} combine with the nitrogen atom to which they are bound to form

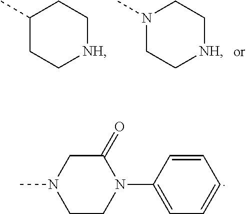

6.-8. (canceled)

9. The compound of claim 1, wherein at least one of the following applies:
(a) R^{1b} is H; and
(b) R² is selected from the group consisting of: 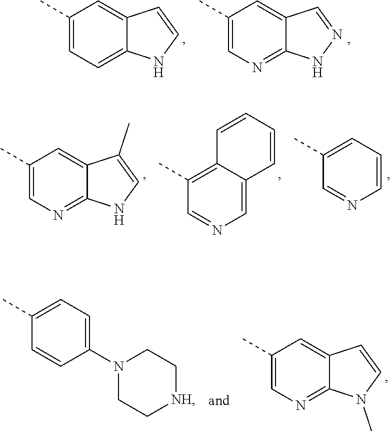
(c) at least one of R^{3a}, R^{3b}, R^{3c}, and R^{3d} is H;
(d) at least two of R^{3a}, R^{3b}, R^{3c}, and R^{3d} are H;
(e) at least three of R^{3a}, R^{3b}, R^{3c}, and R^{3d} are H; and
(f) each of R^{3a}, R^{3b}, R^{3c}, and R^{3d} are H.

10.-11. (canceled)

12. The compound of claim 1, which is selected from the group consisting of:
N-methyl-3-(1H-pyrrolo[2,3-b]pyridin-5-yl)pyrazolo[1,5-a]pyridine-5-carboxamide;
3-(isoquinolin-4-yl)-N-(p-tolyl)pyrazolo[1,5-a]pyridine-5-carboxamide;
N-(4-((methylamino)methyl)phenyl)-3-(1H-pyrrolo[2,3-b]pyridin-5-yl)pyrazolo[1,5-a]pyridine-5-carboxamide;
3-(isoquinolin-4-yl)-N-(4-((methylamino)methyl)phenyl)pyrazolo[1,5-a]pyridine-5-carboxamide;
N-(4-((methylamino)methyl)phenyl)-3-(1H-pyrrolo[2,3-b]pyridin-5-yl)imidazo[1,2-a]pyridine-6-carboxamide;
N-(4-((methylamino)methyl)phenyl)-4-(1H-pyrrolo[2,3-b]pyridin-5-yl)benzo[b]thiophene-2-carboxamide;
N-(3-(dimethylcarbamoyl)-4-methoxyphenyl)-4-(1H-pyrrolo[2,3-b]pyridin-5-yl)benzo[b]thiophene-2-carboxamide;
N-(4-methoxy-3-(piperidine-1-carbonyl)phenyl)-4-(1H-pyrrolo[2,3-b]pyridin-5-yl)benzo[b]thiophene-2-carboxamide;
3-(2-aminopyrimidin-5-yl)-N-(4-((methylamino)methyl)phenyl)pyrazolo[1,5-a]pyridine-5-carboxamide;
N-(4-((dimethylamino)methyl)phenyl)-3-(1H-pyrrolo[2,3-b]pyridin-5-yl)pyrazolo[1,5-a]pyridine-5-carboxamide;
N-(4-(methylcarbamoyl)phenyl)-3-(1H-pyrrolo[2,3-b]pyridin-5-yl)pyrazolo[1,5-a]pyridine-5-carboxamide;
N-(4-fluorophenyl)-3-(1H-pyrrolo[2,3-b]pyridin-5-yl)pyrazolo[1,5-a]pyridine-5-carboxamide;
N-(4-methoxy-3-(morpholine-4-carbonyl)phenyl)-4-(1H-pyrrolo[2,3-b]pyridin-5-yl)benzo[b]thiophene-2-carboxamide;
N-(4-methoxy-3-(4-methylpiperazine-1-carbonyl)phenyl)-4-(1H-pyrrolo[2,3-b]pyridin-5-yl)benzo[b]thiophene-2-carboxamide;
N-(3-(piperidine-1-carbonyl)phenyl)-4-(1H-pyrrolo[2,3-b]pyridin-5-yl)benzo[b]thiophene-2-carboxamide;
N-(4-hydroxy-3-(piperidine-1-carbonyl)phenyl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzo[b]thiophene-2-carboxamide;
N-(3-(dimethylcarbamoyl)-4-methylphenyl)-4-(1H-pyrrolo[2,3-b]pyridin-5-yl)benzo[b]thiophene-2-carboxamide;
N-(3-(dimethylcarbamoyl)-4-methoxyphenyl)-4-(3-methyl-1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzofuran-2-carboxamide;
N-(3-(dimethylcarbamoyl)-4-hydroxyphenyl)-4-(1H-pyrrolo[2,3-b]pyridin-5-yl)benzo[d]thiazole-2-carboxamide;
N-(3-(dimethylcarbamoyl)-4-(trifluoromethoxy)phenyl)-4-(1H-pyrrolo[2,3-b]pyridin-5-yl)benzo[b]thiophene-2-carboxamide;
N-(3-carbamoylphenyl)-4-(1H-pyrrolo[2,3-b]pyridin-5-yl)benzo[b]thiophene-2-carboxamide;
N-(3-(dimethylcarbamoyl)-5-hydroxyphenyl)-4-(1H-pyrrolo[2,3-b]pyridin-5-yl)benzo[b]thiophene-2-carboxamide;
N-(5-(dimethylcarbamoyl)-2-hydroxyphenyl)-4-(1H-pyrrolo[2,3-b]pyridin-5-yl)benzo[b]thiophene-2-carboxamide;
4-(4-(1H-pyrrolo[2,3-b]pyridin-5-yl)benzo[b]thiophene-2-carboxamido)-N,N-dimethylpicolinamide;
N-(4-hydroxy-3-(pyrrolidine-1-carbonyl)phenyl)-4-(1H-pyrrolo[2,3-b]pyridin-5-yl)benzo[b]thiophene-2-carboxamide;
N-(3-(azetidine-1-carbonyl)-4-hydroxyphenyl)-4-(1H-pyrrolo[2,3-b]pyridin-5-yl)benzo[b]thiophene-2-carboxamide;
N-(pyrimidin-5-yl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)benzo[d]thiazole-2-carboxamide;
N-(3-(dimethylcarbamoyl)-4-hydroxyphenyl)-4-(3-methyl-1H-pyrrolo[2,3-b]pyridin-5-yl)benzo[b]thiophene-2-carboxamide;
N-(3-(dimethylcarbamoyl)-4-hydroxyphenyl)-4-(1H-pyrazolo[3,4-b]pyridin-5-yl)benzo[b]thiophene-2-carboxamide;
N-(2-(dimethylamino)ethyl)-4-(1H-pyrrolo[2,3-b]pyridin-5-yl)benzo[b]thiophene-2-carboxamide;
N-(3-((2-(dimethylamino)ethyl)carbamoyl)-4-hydroxyphenyl)-4-(1H-pyrrolo[2,3-b]pyridin-5-yl)benzo[b]thiophene-2-carboxamide;
N-(4-hydroxy-3-(methylcarbamoyl)phenyl)-4-(1H-pyrrolo[2,3-b]pyridin-5-yl)benzo[b]thiophene-2-carboxamide;
N-(3-(dimethylcarbamoyl)-4-hydroxyphenyl)-4-(1H-pyrrolo[2,3-b]pyridin-5-yl)thieno[2,3-c]pyridine-2-carboxamide;
N-(4-hydroxy-3-(oxetan-3-ylcarbamoyl)phenyl)-4-(1H-pyrrolo[2,3-b]pyridin-5-yl)benzo[b]thiophene-2-carboxamide;
N-(3-((cyclopropylmethyl)carbamoyl)-4-hydroxyphenyl)-4-(1H-pyrrolo[2,3-b]pyridin-5-yl)benzo[b]thiophene-2-carboxamide;
N-(3-oxoisoindolin-5-yl)-4-(1H-pyrrolo[2,3-b]pyridin-5-yl)benzo[b]thiophene-2-carboxamide;
4-(4-(1H-pyrrolo[2,3-b]pyridin-5-yl)benzo[b]thiophene-2-carbonyl)-1-phenylpiperazin-2-one;
N-(3-((diethylamino)methyl)-4-hydroxyphenyl)-4-(1H-pyrrolo[2,3-b]pyridin-5-yl)benzo[b]thiophene-2-carboxamide;
N-(3-cyano-4-hydroxyphenyl)-4-(1H-pyrrolo[2,3-b]pyridin-5-yl)benzo[b]thiophene-2-carboxamide;
N-phenyl-3-(1H-pyrrolo[2,3-b]pyridin-5-yl)pyrazolo[1,5-a]pyridine-5-carboxamide;
3-(1H-pyrrolo[2,3-b]pyridin-5-yl)-N-(p-tolyl)pyrazolo[1,5-a]pyridine-5-carboxamide;
3-(pyridin-4-yl)-N-(p-tolyl)pyrazolo[1,5-a]pyridine-5-carboxamide;
3-(1H-indol-5-yl)-N-(p-tolyl)pyrazolo[1,5-a]pyridine-5-carboxamide;
3-(1H-indazol-5-yl)-N-(4-((methylamino)methyl)phenyl)pyrazolo[1,5-a]pyridine-5-carboxamide;
N-(4-((methylamino)methyl)phenyl)-3-(4-(piperazin-1-yl)phenyl)pyrazolo[1,5-a]pyridine-5-carboxamide;
3-(1-methyl-1H-pyrrolo[2,3-b]pyridin-5-yl)-N-(4-((methylamino)methyl)phenyl)pyrazolo[1,5-a]pyridine-5-carboxamide;
3-(1H-pyrrolo[2,3-b]pyridin-5-yl)-N-(4-(trifluoromethyl)phenyl)pyrazolo[1,5-a]pyridine-5-carboxamide;
(3-(1H-pyrrolo[2,3-b]pyridin-5-yl)pyrazolo[1,5-a]pyridin-5-yl)(piperidin-1-yl)methanone;
(3-(1H-pyrrolo[2,3-b]pyridin-5-yl)pyrazolo[1,5-a]pyridin-5-yl)(piperazin-1-yl)methanone;
1-(5-(1H-pyrrolo[2,3-b]pyridin-5-yl)pyridin-3-yl)-N-(4 ((methylamino)methyl)phenyl)cyclopropane-1-carboxamide;
N-(4-((methylamino)methyl)phenyl)-4-(1H-pyrrolo[2,3-b]pyridin-5-yl)benzamide;
N-methyl-4-(1H-pyrrolo[2,3-b]pyridin-5-yl)benzo[b]thiophene-2-carboxamide;
N-phenyl-4-(1H-pyrrolo[2,3-b]pyridin-5-yl)benzo[b]thiophene-2-carboxamide;
N-(4-methoxyphenyl)-4-(1H-pyrrolo[2,3-b]pyridin-5-yl)benzo[b]thiophene-2-carboxamide;
N-(4-methoxy-3-(methylcarbamoyl)phenyl)-4-(1H-pyrrolo[2,3-b]pyridin-5-yl)benzo[b]thiophene-2-carboxamide;
N-(4-methoxy-3-(4-methylpiperazine-1-carbonyl)phenyl)-4-(1H-pyrrolo[2,3-b]pyridin-5-yl)benzo[b]thiophene-2-carboxamide;
N-(3-(dimethylcarbamoyl)phenyl)-4-(1H-pyrrolo[2,3-b]pyridin-5-yl)benzo[b]thiophene-2-carboxamide;
N-(3-(dimethylcarbamoyl)-4-(trifluoromethyl)phenyl)-4-(1H-pyrrolo[2,3-b]pyridin-5-yl)benzo[b]thiophene-2-carboxamide; and
N-(3-(dimethylcarbamoyl)-4-fluorophenyl)-4-(1H-pyrrolo[2,3-b]pyridin-5-yl)benzo[b]thiophene-2-carboxamide.

13. A compound of Formula (II), or a salt, solvate, stereoisomer, or isotopologue thereof: 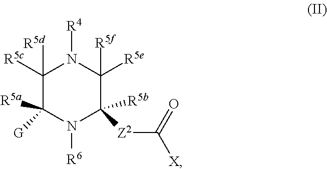
wherein:
R⁴ is selected from the group consisting of H, optionally substituted C₁-C₆ alkyl, optionally substituted C₃-C₈ cycloalkyl, optionally substituted C₂-C₆ heterocycloalkyl, optionally substituted C₇-C₁₂ aralkyl, optionally substituted C₃-C₁₂ heteroaralkyl, optionally substituted C₆-C₁₀ aryl, and optionally substituted C₂-C₁₂ heteroaryl;
R^{5a}, R^{5b}, R^{5c}, R^{5d}, R^{5e}, and R⁵ are each independently selected from the group consisting of H, optionally substituted C₁-C₆ alkyl, halogen, OR^{E}, and N(R^{E})(R^{F});
R⁶ is —(CH₂)₁₋₃C(═O)N(R^{G})(R^{H});
R^{7a} is selected from the group consisting of H and C(═O)OR
R^{7b} is H;
X is selected from the group consisting of OR^{J} and 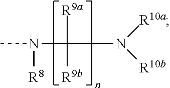
G is selected from the group consisting of optionally substituted C₁-C₆ alkyl and —Z¹—N(R^{7a})(R^{7b});
Z¹ and Z² are each independently-(optionally substituted C₁-C₆ alkylenyl)-;
R⁸ is selected from the group consisting of H and optionally substituted C₁-C₆ alkyl;
R^{9a} and R^{9b} are each independently selected from the group consisting of H and optionally substituted C₁-C₆ alkyl;
R^{10a} and R^{10b} are each independently selected from the group consisting of H, optionally substituted C₁-C₆ alkyl, and optionally substituted C₆-C₁₀ aryl;
n is an integer selected from the group consisting of 1, 2, 3, 4, and 5; and
R^{E}, R^{F}, R^{G}, R^{H}, R^{I}, and R^{J} are each independently selected from the group consisting of H, optionally substituted C₁-C₆ alkyl, optionally substituted C₃-C₈ cycloalkyl, optionally substituted C₁-C₆ heteroalkyl, optionally substituted C₂-C₆ heterocycloalkyl, optionally substituted C₇-C₁₂ aralkyl, optionally substituted C₆-C₁₀ aryl, and optionally substituted C₂-C₁₂ heteroaryl.

14. The compound of claim 13, wherein at least one of the following applies:
(a) R⁴ is selected from the group consisting of H and 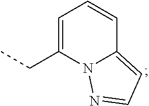
(b) R⁶ is —CH₂C(═O)NHCH₃,
(c) G is CH₃ or —Z¹—N(R^{7a})(R^{7b})
(d) R^{7a} is H or 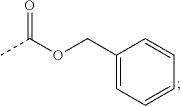
(e) R^{7b} is H;
(f) X is OEt;
(g) Z¹ is —CH₂CH₂CH₂—; and
(h) Z² is —CH₂—.

15. The compound of claim 13, wherein at least one of the following applies:
(a) at least one of R^{5a}, R^{5b}, R^{5c}, R^{5d}, R^{5e}, and R^{5f} is H;
(b) at least two of R^{5a}, R^{5b}, R^{5c}, R^{5d}, R^{5e}, and R^{5f} are H;
(c) at least three of R^{5a}, R^{5b}, R^{5c}, R^{5d}, R^{5e}, and R^{5f} are H;
(d) at least four of R^{5a}, R^{5b}, R^{5c}, R^{5d}, R^{5e}, and R^{5f} are H;
(e) at least five of R^{5a}, R^{5b}, R^{5c}, R^{5d}, R^{5e}, and R^{5f} are H; and
(f) each of R^{5a}, R^{5b}, R^{5c}, R^{5d}, R^{5e}, and R^{5f} are H.

16.-21. (canceled)

22. The compound of claim 13, wherein X is

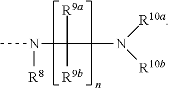

23. The compound of claim 22, wherein at least one of the following applies:
(a) each occurrence of R^{9a} and R^{9b} are independently H;
(b) n is 3;
(c) R^{10a} and R^{10b} are each independently selected from the group consisting of 4-fluorophenyl and methyl.

24.-25. (canceled)

26. The compound of claim 13, wherein X is

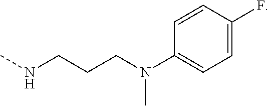

27.-28. (canceled)

29. The compound of claim 13, which is selected from the group consisting of:
benzyl (2-((2R,6R)-6-(2-((3-((4-fluorophenyl)(methyl)amino)propyl)amino)-2-oxoethyl)-1-(2-(methylamino)-2-oxoethyl)-4-(pyrazolo[1,5-a]pyridin-7-ylmethyl)piperazin-2-yl)ethyl)carbamate;
ethyl 2-((2R,6R)-6-(2-(((benzyloxy)carbonyl)amino)ethyl)-1-(2-(methylamino)-2-oxoethyl)-4-(pyrazolo[1,5-a]pyridin-7-ylmethyl)piperazin-2-yl)acetate;
benzyl (2-((2R,6R)-6-(2-((3-((4-fluorophenyl)(methyl)amino)propyl)amino)-2-oxoethyl)-1-(2-(methylamino)-2-oxoethyl)piperazin-2-yl)ethyl)carbamate;
N-(3-((4-fluorophenyl)(methyl)amino)propyl)-2-((2R,6R)-6-methyl-1-(2-(methylamino)-2-oxoethyl)piperazin-2-yl)acetamide.

30. A pharmaceutical composition comprising at least one compound of claim 1 and a pharmaceutically acceptable carrier, optionally wherein the composition further comprises at least one therapeutically effective agent.

31. (canceled)

32. A method of treating, preventing, and/or ameliorating an inflammatory disease in a subject, the method comprising administering to the subject a therapeutically effective amount of at least one compound of claim 1 or a pharmaceutical composition thereof.

33. The method of claim 32, wherein JNK1 is inhibited at a similar or greater rate of inhibition than JNK2 and/or JNK3.

34. (canceled)

35. The method of claim 32, wherein at least one of the following applies:
(a) the inflammatory disease is a non-central nervous system (CNS) inflammatory disease,
(b) the inflammatory disease is at least one selected from the group consisting of endometriosis, arthritis, pulmonary fibrosis, cancer, type 1 diabetes, and type 2 diabetes;
(c) the inflammatory disease is endometriosis;
(d) progesterone insensitivity in the subject is reduced and/or eliminated;
(e) pain associated with endometriosis is reduced and/or eliminated;
(f) the inflammatory disease is selected from the group consisting of Alzheimer's disease, Parkinson's disease, and amyotrophic lateral sclerosis; and
(g) wherein normal reproductive function is maintained in the subject.

36.-40. (canceled)

41. The method of claim 32, wherein the subject is further administered progesterone.

42. (canceled)

43. The method of claim 32, wherein the method further comprises detecting the inflammatory disease or disorder in the subject, optionally wherein the detecting comprises administration of a suitable diagnostic method.

44. (canceled)

45. The method of claim 32, wherein the subject is a mammal, optionally wherein the mammal is a human, and optionally wherein the human is a female.

46.-47. (canceled)
